(19) United States
(12) Patent Application Publication
(10) Pub. No.: US 2007/0185028 A1
(43) Pub. Date: Aug. 9, 2007

(54) Compositions and methods for treatment of protein misfolding and protein aggregation diseases

(76) Inventors: Joy G. Ghosh, Seattle, WA (US); John I. Clark, Seattle, WA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/607,519

(22) Filed: Dec. 1, 2006

Related U.S. Application Data
(63) Continuation-in-part of application No. PCT/US05/40161, filed on Nov. 4, 2005.
(60) Provisional application No. 60/625,364, filed on Nov. 4, 2004.
(60) Provisional application No. 60/724,961, filed on Oct. 7, 2005.

(51) Int. Cl. A61K 38/16; A61K 38/10; A61K 38/08; C07K 7/08; C07K 14/47

(52) U.S. Cl. 514/12; 514/13; 514/14; 514/15; 514/16; 530/324; 530/325; 530/326; 530/327; 530/328; 530/329

(57) ABSTRACT

Small molecular weight molecules are provided including, but not limited to, peptides, peptide analogs and peptide mimetics that stabilize and prevent the aggregation of abnormally folded and compromised proteins. Methods for treatment of disease are provided utilizing the peptides, peptide analogs or peptide mimetics, or utilizing nucleic acids encoding the peptides.

### CROSS REFERENCE TO RELATED APPLICATIONS

This application is a continuation-in-part application of PCT Application No. PCT/US2005/040161, filed Nov. 4, 2005, which relates to U.S. Provisional Application No. 60/625,364, filed Nov. 4, 2004. and U.S. Provisional Application No. 60/724,961, filed Oct. 7, 2005, which are each incorporated herein by reference in their entireties.

### STATEMENT OF GOVERNMENT SUPPORT

This invention was made by government support by Grant No. EY04542 from National Eye Institute of The National Institutes of Health. The Government has certain rights in this invention.

### FIELD

The present invention is directed to small molecular weight molecules including, but not limited to, peptides, peptide analogs and peptide mimetics that stabilize the non-native states of proteins and prevent the aggregation of unfolded, abnormally folded or misfolded proteins. The invention is further directed to methods for treatment of protein conformation disease utilizing the peptides, peptide analogs or peptide mimetics, or nucleic acids encoding the peptides.

### BACKGROUND

Proteins are large polypeptide chains composed of sequences of amino acids encoded by genes and synthesized by the protein synthesis machinery of cells. Synthesis of proteins is followed by folding into functional 3-dimensional structures, which often requires participation of separate proteins called molecular chaperones. Molecular chaperones are endogenous specialized proteins that assist normal folding of synthesized polypeptides into their functional form. Correctly folded proteins are transported to their destination where they perform their function(s).

Mutations, molecular and environmental stress, post-translational modifications, proteolysis and aging can alter the structure of a protein leading to an unfolding or misfolded protein with an altered function. The altered function can lead to increased morbidity through a number of mechanisms including, but not limited to, disruption of important cellular processes, toxicity due to aggregation and cell-death responses.

The interaction between molecular chaperones and partially folded polypeptides is a natural defense against protein unfolding and aggregation diseases. Protein conformation diseases occur when natural proteins in the body gain or lose function due to structural instability. Protein aggregation diseases are a subtype of protein conformation diseases in which unfolded or misfolded proteins form aggregates that are toxic to cells. A large number of protein conformation diseases are a natural consequence of aging. With age, the ability of cells to protect themselves from the lethal effects of protein unfolding and aggregation diminishes greatly. The ability of molecular chaperones which are the natural defense molecules against protein unfolding and misfolding reduces dramatically with age while the number of unfolded and misfolded proteins increases dramatically. Consequently, when protective pathways like refolding and degradation become inefficient and are unable to clear non-functional structurally compromised proteins from cells and/or tissues, the result is protein misfolding and aggregation diseases (Table 1). Sanders and Myers, Annu. Rev. Biophys. Biomol. Struct., 33: 25-51, 2004.

TABLE 1
List of protein conformation diseases and the respective etiological
proteins that have been implicated in those diseases.

| Disease/Pathology | Aggregation-prone proteins |
|---|---|
| Neurodegenerative pathologies | |
| Alzheimer's disease | Amyloid beta, tau |
| Parkinson's disease | Alpha-synuclein, tau |
| Creutzfeld-Jakob disease | Amyloid protein |
| Kuru | Amyloid protein |
| GSS disease | Amyloid protein |
| Huntington's disease | Huntingtin |
| Polyglutamine diseases | Atrophin-1, ataxins |
| Prion disease | Prion protein |
| Bovine Spongiform Encephalopathy | Prion protein |
| (BSE) | |
| Amyotrophic Lateral Sclerosis | Superoxide dismutase |
| Alexander's disease | Glial fibrillary acidic protein |
| Primary Systemic Amyloidosis | Immunoglobulin light chain or |
| | fragments |
| Secondary Systemic Amyloidosis | Fragments of serum amyloid-A |
| Senile Systemic Amyloidosis | Transthyretin and fragments |
| Amyloidosis in senescence | Apolipoprotein A-II |
| Ocular pathologies | |
| Cataract | Crystallins, filaments |
| Retinitis Pigmentosa | Rhodopsin |
| Macular Degeneration | Amyloid-beta, crystallins |
| Other pathologies | |
| Islet amyloid | Insulin |
| Medullar Carcinoma of the Thyroid | Calcitonin |
| Hereditary Renal Amyloidosis | Fibrinogen |
| Hemodialysis-related amyloidosis | beta 2-microglobulin |
| Desmin-related Cardiomyopathy | Desmin |

While molecular chaperones can consist of thousands of peptides, only a small proportion of the peptides are necessary for their function against protein conformation diseases. Santhoshkumar and Sharma, Molecular and Cellular Biochemistry 267: 147-155, 2004. Although protein molecular chaperones are very efficient in vivo, their enormous size limits their bioavailability in therapeutic applications. Accordingly, there is a clear and unmet need in the art for peptides having the functional characteristics of molecular chaperones, which may be more readily produced and used in a variety of therapeutic and manufacturing applications.

### SUMMARY

The present invention generally relates to polypeptides, peptide analogs and peptide mimetics that stabilize and reduce the aggregation of unfolded, abnormally folded or misfolded proteins. Accordingly, the present invention provides peptide-based compositions, peptide variant compositions, or peptide mimetic compositions that inhibit protein misfolding and/or aggregation and are, therefore, useful in a variety of therapeutic and manufacturing applications, including, e.g., the treatment of diseases and disorders associated with protein misfolding and/or aggregation and methods for manufacturing and purifying recombinant proteins. The present invention provides polypeptide compositions, functional variants, and peptide mimetics thereof, and methods for treating a disease in a mammalian subject comprising administering a polypeptide up to about 50 amino acids in length having molecular chaperone activity to the subject in need thereof. The methods are useful for treating diseases, for example, diseases related to protein aggregation, and diseases such as age-related myopathy and cardiac ischemia. A method for stabilizing a protein is provided comprising contacting the protein with a polypeptide up to about 50 amino acids in length having molecular chaperone activity. A method is provided to increase the efficacy of a therapeutic protein to treat disease. A method is also provided to increase production of a recombinantly-produced protein. The methods provide a polypeptide up to about 50 amino acids in length that limits protein aggregation and provides recombinant proteins with correct folding of the polypeptide as an active protein compositions.

A polypeptide X₁-HGKHEERQDE-X₂, or a functional variant or mimetic thereof is provided wherein each X₁ and X₂ independently of one another represents any amino acid sequence of n amino acids, n varying from 0 to 50, and n being identical or different in X₁ and X₂. The functional variant or mimetic can be a conservative amino acid substitution or peptide mimetic substitution. In one aspect, the functional variant has about 70% or greater amino acid sequence identity to X₁-HGKHEERQDE-X₂. The functional variant can include, but is not limited to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The functional variant can have about 70% or greater amino acid sequence identity to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂.

A method for treating a protein conformation disease in a mammalian subject is provided which comprises administering a polypeptide to the subject in need thereof, wherein the polypeptide is X₁-HGKHEERQDE-X₂, or a functional variant or mimetic thereof, wherein each X₁ and X₂ independently of one another represents any amino acid sequence of n amino acids, n varying from 0 to 50, and n being identical or different in X₁ and X₂. The functional variant or mimetic can be a conservative amino acid substitution or peptide mimetic substitution. In one aspect, the functional variant has about 70% or greater amino acid sequence identity to X₁-HGKHEERQDE-X₂. The functional variant can include, but is not limited to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The functional variant can have about 70% or greater amino acid sequence identity to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The disease includes, but is not limited to, Alexander's disease, Alzheimer's disease, Creutzfeld-Jakob disease, Parkinson's disease, senile systemic amyloidosis, hemodialysis-related amyloidosis, Huntington's disease, cataract, retinitis pigmentosa, prion disease, or mad cow disease. The disease includes, but is not limited to, age-related myopathy or cardiac ischemia.

A method for stabilizing a protein is provided which comprises contacting the protein with a polypeptide X₁-HGKHEERQDE-X₂, or a functional variant or mimetic thereof, wherein each X₁ and X₂ independently of one another represents any amino acid sequence of n amino acids, n varying from 0 to 50, and n being identical or different in X₁ and X₂. The functional variant or mimetic can be a conservative amino acid substitution or peptide mimetic substitution. In one aspect, the functional variant has about 70% or greater amino acid sequence identity to X₁-HGKHEERQDE-X₂. The functional variant can include, but is not limited to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The functional variant can have about 70% or greater amino acid sequence identity to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The protein can be a therapeutic protein. The therapeutic protein includes, but is not limited to, a vaccine, insulin, growth factor, or antibody. The protein can be a recombinantly-produced protein. The method further comprises increasing the stability of the therapeutic protein to treat a disease state in a mammalian subject. The method further comprises inhibiting protein misfolding or reducing protein aggregation. The method further comprises restoring correct or native folding to the protein.

A method for diagnosing a protein conformation disease in a mammalian subject comprising contacting a tissue sample from the mammalian subject with a polypeptide X₁-HGKHEERQDE-X₂, or a functional variant or mimetic thereof, wherein each X₁ and X₂ independently of one another represents any amino acid sequence of n amino acids, n varying from 0 to 50, and n being identical or different in X₁ and X₂, detecting binding of a misfolded protein, an unfolded protein, or an aggregated protein to the polypeptide, and determining a presence or absence of the disease in the mammalian subject. The functional variant or mimetic can be a conservative amino acid substitution or peptide mimetic substitution. In one aspect, the functional variant has about 70% or greater amino acid sequence identity to X₁-HGKHEERQDE-X₂. The functional variant can include, but is not limited to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The functional variant can have about 70% or greater amino acid sequence identity to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. In the method, the presence or absence of the disease is detected by a stage of misfolding, unfolding, or aggregation of the protein. The protein conformation disease includes, but is not limited to, Alexander's disease, Alzheimer's disease, Creutzfeld-Jakob disease, Parkinson's disease, senile systemic amyloidosis, hemodialysis-related amyloidosis, Huntington's disease, cataract, retinitis pigmentosa, prion disease, or mad cow disease. The protein conformation disease includes, but is not limited to, age-related myopathy or cardiac ischemia.

An in vitro method of screening for a modulator of protein misfolding, protein unfolding, or protein aggregation activity is provided which comprises contacting a target protein with a polypeptide X₁-HGKHEERQDE-X₂, or a functional variant or mimetic thereof, wherein each X₁ and X₂ independently of one another represents any amino acid sequence of n amino acids, n varying from 0 to 50, and n being identical or different in X₁ and X₂, and detecting a decrease in the amount of protein aggregation activity; thereby identifying the polypeptide as a modulator of protein misfolding, protein unfolding, or protein aggregation activity.

An in vivo method of screening for a modulator of protein misfolding, protein unfolding, or protein aggregation activity is provided which comprises contacting a cell or cell line expressing a target protein with a test compound encoding a polypeptide X₁-HGKHEERQDE-X₂, or a functional variant or mimetic thereof, wherein each X₁ and X₂ independently of one another represents any amino acid sequence of n amino acids, n varying from 0 to 50, and n being identical or different in X₁ and X₂, and detecting a decrease in the amount of protein aggregation activity in the cell line, thereby identifying the polypeptide as a modulator of protein misfolding, protein unfolding, or protein aggregation activity.

The target protein includes, but is not limited to, amyloid-beta, beta/gamma crystallins, actin, desmin, vimentin, insulin, citrate synthase, alcohol dehydrogenase, glial fibrillary acidic protein, alpha-lactalbumin, fibroblast growth factor, insulin-like growth factor, transforming growth factor-beta, nerve growth factor-beta, epidermal growth factor, vascular endothelial growth factor, beta-catenin, tumor necrosis factor-alpha, Bcl-2, Bcl-X1 or caspase.

The functional variant can have about 70% or greater amino acid sequence identity to X₁-HGKHEERQDE-X₂. The functional variant includes, but is not limited to, X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂. The functional variant can have about 70% or greater amino acid sequence identity to X₁-HGKH-X₂, X₁-KHEE-X₂, X₁-EERQDE-X₂, X₁-RQDE-X₂, X₁-EERQ-X₂, X₁-HGKHEE-X₂, X₁-KHEERQ-X₂, X₁-HEER-X₂, X₁-HGKHEERQ-X₂, or X₁-KHEERQDE-X₂.

### BRIEF DESCRIPTION OF THE DRAWINGS

FIG. 1 is a diagram depicting therapeutic applications for Intellipeptides that stabilize misfolded proteins and/or prevent the aggregation of proteins.

FIG. 2 is a bar graph depicting the effect of Intellipeptides on the pH induced aggregation of amyloid-beta in vitro.

FIG. 3 is a bar graph depicting the effect of Intellipeptides on the Cu⁺⁺⁺ induced aggregation of amyloid-beta in vitro.

FIG. 4 shows a schematic diagram of amyloidosis in Alzheimer's disease (AD) and Parkinson's disease (PD) and possible interventions using Intellipeptides.

FIG. 5 shows a method for using a molecular model of an electrostatic surface to design a synthetic molecule with characteristics of a polypeptide.

FIG. 6 shows a summary of a series of peptides derived from polypeptide sequences from polypeptides SLSPFYLRPPSFLRAPS, EKDRFSVNLDVKHFS, HGFISREFHRKYR, DPLTITSSLSSDGVLTVNGPRKQ, and PERTIPITREEK.

FIG. 7 shows the amino acid sequence of a series of peptides derived from the sequence SLSPFYLRPPSFLRAPS.

FIG. 8 shows the amino acid sequence of a series of peptides derived from the sequence EKDRFSVNLDVKHFS.

FIG. 9 shows the amino acid sequence of a series of peptides derived from the sequence HGFISREFHRKYR.

FIG. 10 shows the amino acid sequence of a series of peptides derived from the sequence DPLTITSSLSSDGVLTVNGPRKQ.

FIG. 11 shows the amino acid sequence of a series of peptides derived from the sequence PERTIPITREEK.

FIG. 12 shows a schematic for the protein pin array assay. Refer to the methods section for detailed protocols.

FIG. 13 shows a pattern of interactions between human αB crystallin 8-mer peptides immobilized on pins and unheated β_{H} crystallin at 23° C. and β_{H} crystallin pre-heated at 45° C. for fifteen minutes.

FIG. 14 shows a pattern of interactions between human αB crystallin 8-mer peptides immobilized on pins and unheated γD crystallin at 23° C. and γD crystallin pre-heated at 45° C. for fifteen minutes.

FIG. 15 shows a far UVCD of β_{H} crystallin, γD crystallin, alcohol dehydrogenase (ADH) and citrate synthase (CS). Spectra were collected for β_{H} crystallin (A: top left), γD crystallin (B: top right), ADH (C: bottom left) and CS (D: bottom right), at 23° C., 45° C. and 50° C.

FIG. 16 shows a near UVCD of β_{H} crystallin, γD crystallin, ADH and CS. Spectra were collected for β_{H} crystallin (A: top left), γD crystallin (B: top right), ADH (C: bottom left) and CS (D: bottom right) at 23° C., 45° C. and 50° C.

FIG. 17 shows a pattern of interaction between human αB crystallin peptides and ADH.

FIG. 18 shows a pattern of interaction between human αB crystallin peptides and CS.

FIG. 19 shows a chaperone assays of two positive interactive sequences, ₇₃DRFSVNLDVKHFS₈₅ and ₁₃₁LTITSSLSDGV₁₄₁ and a non-interactive sequence, ₁₁₁HGKHEERQDE₁₂₀ from the α crystallin core domain of human αB crystallin (control).

FIG. 20 shows a comparison of the peptides identified using the human αB crystallin pin arrays with previously reported interactive sequences for αB crystallin.

FIG. 21 shows a 3-dimensional map of the αB crystallin interactive domains.

FIG. 22 shows the effect of αB crystallin and five αB crystallin derived peptides on the fibrillization of Aβ.

FIG. 23 shows the effect of αB crystallin and five αB crystallin derived peptides on the fibrillization of γD crystallin.

FIG. 24 shows chaperone assays of the five αB crystallin derived peptides.

FIG. 25 shows an effect of five βB crystallin peptides on Aβ₁₋₄₂ fibril formation.

FIG. 26 shows an effect of five αB crystallin peptides on transthyretin fibril formation.

FIG. 27 shows an effect of five αB crystallin peptides on β2-microglobulin fibril formation.

FIG. 28 shows an effect of five αB crystallin peptides on α-synuclein fibril formation.

FIG. 29 shows an effect of HG derived peptides on TTR fibrillation.

FIG. 30 shows an effect of HG derived peptides on β2m fibrillation.

FIG. 31 shows an effect of HG derived peptides on α-synuclein fibrillation.

FIGS. 32A, 32B, and 32C show a hypothetical mechanism by which αB crystallin peptides inhibit the fibrillation of amyloidogenic proteins.

FIG. 33 shows a library of peptides that were derived using the HGKHEERQDE peptide.

### DETAILED DESCRIPTION

The present invention provides polypeptides, peptide analogs and peptide mimetics the non-native states of proteins and prevent the aggregation of unfolded, abnormally folded or misfolded proteins. Accordingly, the present invention provides peptide-based compositions that inhibit protein misfolding, abnormal folding, and/or aggregation and are, therefore, useful in a variety of therapeutic and manufacturing applications, including, e.g., the treatment of diseases and disorders associated with protein misfolding, abnormal folding and/or aggregation and in methods for manufacturing and purifying recombinant proteins.

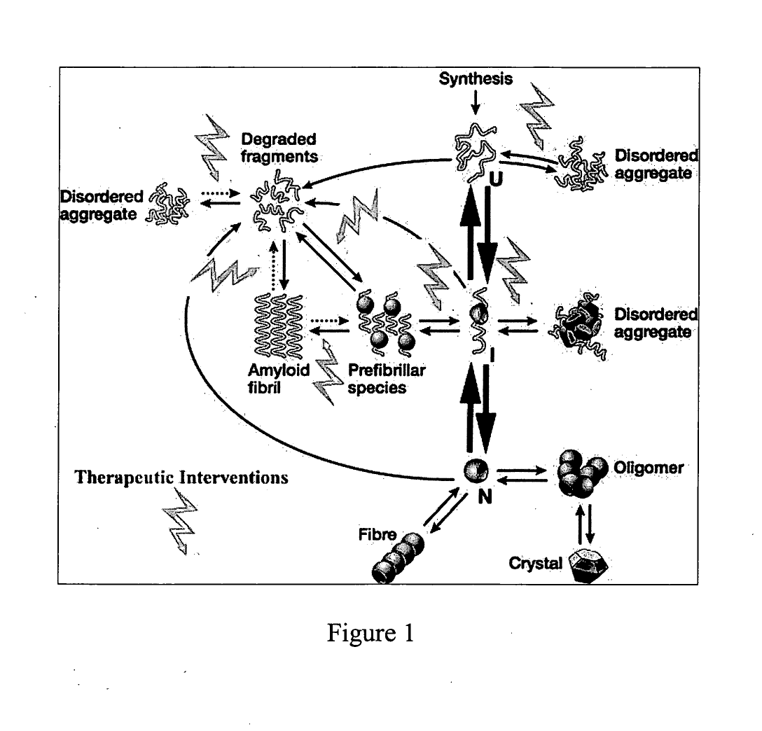

FIG. 1 is a diagram depicting therapeutic applications for Intellipeptides that stabilize misfolded proteins and/or prevent the aggregation of proteins. A schematic of the normal pathway for a newly synthesized protein (U), partially folded intermediate (I) and completely folded native protein (N) is shown using bold continuous arrows. Thin arrows in the figure represent pathways for protein aggregation and degradation. Potential sites of therapeutic intervention in which Intellipeptides can intervene to prevent protein misfolding and aggregation disease pathologies are depicted as lightning bolts.

Protein pin arrays identified interactive polypeptide sequences for chaperone activity in human αB crystallin using natural lens proteins, β_{H} crystallin and γD crystallin, and in vitro chaperone target proteins, for example, alcohol dehydrogenase and citrate synthase. A polypeptide fragment having activity to stabilize and reduce aggregation of misfolded proteins comprises polypeptide sequences from the N-terminal domain, α crystallin core domain, or the C-terminal domain of the human αB crystallin protein. The N-terminal domain contained interactive polypeptide sequences with chaperone activity, ₉WIRRPFFPFHSP₂₀ and ₄₃SLSPFYLRPPSFLRAP₅₈. The N-terminal domain also contained the following interactive polypeptide sequences with chaperone activity: WIRRPFFP, PFFPFHSP, FPFHSPSR, DQFFGEHL, FFGEHLLE, or IAIHHPWI, SPFYLRPP, SLSPFYLR, FYLRPPSF, LRPPSFLR, PPSFLRAP, SFLRAPSW, LRAPSWFD. The α crystallin core domain contained interactive protein sequences with chaperone activity, ₇₅FSVNLDVK₈₂ (β3), ₁₁₃FISREFHR₁₂₀, ₁₃₁LTITSSLS₁₃₈ (β8) and ₁₄₁GVLTVNGP₁₄₈ (β9). The α crystallin core domain also contained interactive protein sequences with chaperone activity: RLEKDRFS, LKVKVLGD, HGFISREF, KYRIPADV, EFHRKYRI, SREFHRKY, or LTVNGPRK. The C-terminal domain contained an interactive sequence, ₁₅₇RTIPITRE₁₆₄ that included the highly conserved I-X-I/V amino acid motif. Two interactive sequences, ₇₃DRFSVNLDVKHFS₈₅ and ₁₃₁LTITSSLSDGV₁₄₁ belonging to the α crystallin core domain were synthesized as peptides and assayed for chaperone activity in vitro. Both synthesized peptides inhibited the thermal aggregation of β_{H} crystallin, alcohol dehydrogenase and citrate synthase in vitro. Five of the seven chaperone sequences identified by the pin arrays overlapped with sequences identified previously as sequences for subunit-subunit interactions in human αB crystallin. The results suggested that interactive sequences in human αB crystallin have dual roles in subunit-subunit assembly and chaperone activity.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

Peptides

The peptides, peptide analogs and peptide-mimetics of the present invention, herein are collectively referred to as “Intellipeptides”, “aggregation inhibition peptides”, “peptides that inhibit abnormal protein folding, protein unfolding, protein misfolding, or protein aggregation.” Intellipeptides are identified using protein pin arrays, computer modeling, multiple sequence alignment analyses of structurally and functionally similar proteins, spectroscopic in vitro chaperone assays and/or in vivo cell killing assays.

Intellipeptides stabilize and prevent the protein unfolding, misfolding or aggregation of a wide variety of target proteins including, but not limited to, amyloid-beta, beta/gamma crystallins, actin, desmin, vimentin, insulin, citrate synthase, alcohol dehydrogenase, glial fibrillary acidic protein, alpha-lactalbumin, fibroblast growth factor, insulin-like growth factor, transforming growth factor-beta, nerve growth factor-beta, epidermal growth factor, vascular endothelial growth factor, beta-catenin, tumor necrosis factor-alpha, Bcl-2, Bcl-X1 and caspases.

In a method for treating a protein conformation disease in a mammalian subject, Intellipeptides are useful stabilize and or prevent the protein unfolding, misfolding or aggregation of a wide variety of disease target proteins. Disease targetin proteins include, but not limited to, neurodegenerative disease: Alzheimer's disease (Amyloid beta, tau); Parkinson's disease (Alpha-synuclein, tau); Creutzfeld-Jakob disease (Amyloid protein); Kuru (Amyloid protein); GSS disease (Amyloid protein); Huntington's disease (Huntingtin); Polyglutamine diseases (Atrophin-1, ataxins); Prion disease (Prion protein); Bovine Spongiform Encephalopathy (BSE) (Prion protein); Amyotrophic Lateral Sclerosis (Superoxide dismutase); Alexander's disease (Glial fibrillary acidic protein); Primary Systemic Amyloidosis (Immunoglobulin light chain or fragments); Secondary Systemic Amyloidosis (Fragments of serum amyloid-A); Senile Systemic Amyloidosis (Transthyretin and fragments); Amyloidosis in senescence (Apolipoprotein A-II); Ocular disease: Cataract (Crystallins, filaments); Retinitis Pigmentosa (Rhodopsin); Macular Degeneration (Amyloid-beta, crystallins); and other disease: Islet amyloid (Insulin); Medullar Carcinoma of the Thyroid (Calcitonin); Hereditary Renal Amyloidosis (Fibrinogen); Hemodialysis-related amyloidosis (beta 2-microglobulin); Desmin-related Cardiomyopathy (Desmin); Charcot-Marie Tooth disease (PMP-22); diabetes insipidis (aquaporin); diabetes insipidis (vasopressin receptor); Charcot-Marie Tooth disease (connexin 32); cystic fibrosis (CFTR). See, for example, Sanders and Myers, Annu. Rev. Biophys. Biomol. Struct., 33: 25-51, 2004.

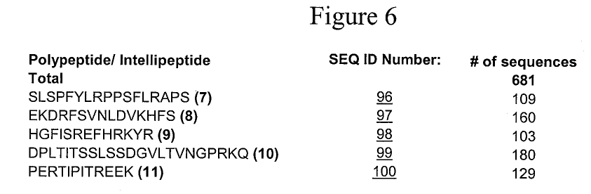

In one embodiment, Intellipeptides of the present invention comprise or consist of a fragment of αB crystallin. In another embodiment, Intellipeptides of the present invention comprise or consist of peptides that are structurally and functionally similar to the parent set of peptide sequences identified from αB crystallin, including, but not limited to the peptides provided in FIGS. 6, 7, 8, 9, 10 and 11 and Table 4. The present invention demonstrates that the parent set and peptide analogs and peptide mimetics of the parent set of these sequences interfere with the interaction between misfolded or unfolded subunits, inhibiting the formation of protein aggregates. In addition, Intellipeptides stabilize misfolded or unfolding intermediates by providing a protective environment conducive to refolding.

In certain embodiments, Intellipeptides include peptide analogs and peptide mimetics. Indeed, Intellipeptides include peptides having any of a variety of different modifications, including those described herein.

Intellipeptide analogs are generally designed and produced by chemical modifications of a lead peptide, including, e.g., any of the particular peptides described herein, such as any of the following sequences: i) EKDRFSVNLDVKHFS; ii) DPLTITSSLSSDGVLTVNGPRKQ; iii) LTITSSLSDGVLTVNGPRK; iv) STSLSPFYLRPPSFLRAP; v) SLSPFYLRPPSFLRAPS; vi) GPERTIPITREEK; vii) PERTIPITREEK; viii) HGKHEERQDE; ix) HGFISREFHRKYR or functional variants or peptide mimetics thereof. An exemplary polypeptide fragment of αB crystallin protein having molecular chaperone activity is presented; e.g., the N-terminal domain polypeptide fragment is ₉WIRRPFFPFHSP₂₀ or ₄₃SLSPFYLRPPSFLRAP₅₈, the α crystallin core domain polypeptide fragment is ₇₅FSVNLDVK₈₂ (β3), ₁₁₃FISREFHR₁₂₀, ₁₃₁LTITSSLS₁₃₈ (β8), ₁₄₁GVLTVNGP₁₄₈ (β9), ₇₃DRFSVNLDVKHFS₈₅, or ₁₃₁LTITSSLSDGV₁₄₁, or the C-terminal domain polypeptide fragment is ₁₅₇RTIPITRE₁₆₄, or functional variants thereof. The present invention clearly establishes that these peptides in their entirety and derivatives created by modifying any side chains of the constituent amino acids have the ability to prevent aggregation of proteins, correctly fold proteins, and stabilize proteins. The present invention further encompasses polypeptides up to about 50 amino acids in length that include the amino acid sequences and functional variants or peptide mimetics of the sequences described herein.

In one embodiment, an Intellipeptide of the present invention includes an N- and C-terminal modification. N-terminal acetylation or desamination confers protection against digestion by a number of aminopeptidases in the presence of amides or alcohols replacing the C-terminal carboxyl group prevent splitting by several carboxypeptidases, including carboxypeptidases A and B.

In another embodiment, an Intellipeptide of the present invention includes a side-chain modification. The presence of non-natural amino acids usually increases peptide stability. In addition, at least one of these amino acids (alpha-aminoisobutyric acid or Aib) imposes significant constraints to model peptides diminishing their conformational flexibility. Therefore, the introduction of Aib is expected to enhance peptide stability and inhibitory activity at the same time.

In a further embodiment, an Intellipeptide of the present invention includes modifications in the alpha-carbon. The most commonly used alpha-carbon modification to improve peptide stability is alpha-methylation. In addition, replacement of the hydrogen atom linked to the alpha-carbon of Phe, Val or Leu favors the adoption of beta-bend conformation that is unfavorable for the formation of beta-pleated sheet structures. According to the present invention, methylation of those residues in the inhibitor peptides is expected to enhance stability and potency.

In another embodiment, an Intellipeptide of the present invention includes a chirality change. Replacement of the natural L-residue by the D-enantiomers dramatically increases resistance to proteolytic degradation. Aggregation inhibitors containing D-enantiomers are as effective in preventing aggregation as the L-enantiomer forms of the aggregation inhibition parent peptides.

In another embodiment, Intellipeptides of the present invention are cyclic peptides. Conformationally constrained cyclic peptides represent better drug candidates than linear peptides due to their reduced conformational flexibility and improved resistance to exopeptidase cleavage. Two alternative strategies can be used to convert a linear sequence into a cyclic structure. One is the introduction of cysteine residue to achieve cyclization through the formation of a disulfide bridge and the other is the side-chain attachment strategy involving resin-bound head-to-tail cyclization. To avoid modifications of the peptide sequence the latter approach is used. Aggregation inhibition peptides contain the ideal sequences for facilitating macrocyclization because proline, due to its ability to promote turns and loops, is a constituent of many naturally occurring or artificially synthesized cyclic peptides.

In another embodiment, an Intellipeptide of the present invention is a pseudopeptides. Pseudopeptides or amide bond surrogates refers to peptides containing chemical modifications of some (or all) of the peptide bonds. The introduction of amide bond surrogates not only decreases peptide degradation but also may significantly modify some of the biochemical properties of the peptides, particularly the conformational flexibility and hydrophobicity. It is likely that an increase in conformational flexibility will be beneficial for docking the inhibitor to the binding sites. On the other hand, since the interaction between the aggregation-prone proteins and the inhibitors seems to depend to a great extent on hydrophobic interactions, it is likely that amide bond replacement increasing hydrophobicity may enhance affinity and hence, potency of the inhibitors. In addition, increased hydrophobicity could also enhance transport of the peptide across membranes and thus, improve barrier permeability (blood-brain barrier and intestinal barrier). The amide bonds to replace are those located at the end of the peptide to prevent exoprotease degradation and after each of the prolines, since it has been described that a frequent endopeptidase cleavage site occurs after this residue by an enzyme known as prolylendopeptidase.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers or recombinantly though heterologous polypeptides such as Fc regions.

It is known in the art that one or more amino acids can be deleted from the N-terminus or C-terminus without substantial loss of biological function. See, e.g., Ron, et al., Biol. Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial chaperone polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the PEDF-R polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific embodiment of the invention includes chaperone polypeptides that include one or more amino acid substitutions that mimic modified amino acids at positions where amino acids that are capable of being modified are normally positioned. Further included are chaperone polypeptides where any subset of modifiable amino acids is substituted. For example, a chaperone polypeptide that includes three serine residues can be substituted at any one, any two, or all three of said serines. Furthermore, any chaperone polypeptide amino acid capable of being modified can be excluded from substitution with a modification-mimicking amino acid.

The present invention is further directed to fragments of the polypeptides of the present invention. More specifically, the present invention embodies purified, isolated, and recombinant polypeptides comprising at least any one integer between 6 and 504 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35, 40, 50, 60, 75, 100, 125, 150, 175, 200, 225, 250, 275, 300, 325, 350, 360, or more consecutive amino acids of a polypeptide of the present invention.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, can be excluded.

In addition, it should be understood that in certain embodiments, Intellipeptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

The present invention includes libraries of Intellipeptides. Such libraries include both peptide libraries and libraries of nucleic acid constructs capable of expressing Intellipeptides. In one embodiment, a library of the present invention consists of sequences related to i) EKDRFSVNLDVKHFS; ii) DPLTITSSLSSDGVLTVNGPRKQ; iii) LTITSSLSDGVLTVNGPRK; iv) STSLSPFYLRPPSFLRAP; v) SLSPFYLRPPSFLRAPS; vi) GPERTIPITREEK; vii) PERTIPITREEK; viii) HGKHEERQDE; ix) HGFISREFHRKYR or functional derivatives or mimetics thereof. In a particular embodiment, a library of the invention consists of two or more Intellipeptides or encoding sequences, including, e.g., the sequences provided in FIGS. 6, 7, 8, 9, 10, and 11, and Table 4.

Peptides, Peptide Variants, and Peptide Mimetics

The invention provides isolated or recombinant polypeptides comprising an amino acid sequence having at least 95%, 96%, 97%, 98%, 99% or more sequence identity to a polypeptide fragment of an N-terminal domain, an α crystallin core domain, or a C-terminal domain of the αB crystallin protein over a region of at least about 10, 50, 100, 150, or 200, or more residues, or, a polypeptide encoded by a nucleic acid of the invention. In one aspect, the polypeptide comprises a sequence as set forth in a polypeptide fragment of an N-terminal domain, an α crystallin core domain, or a C-terminal domain of the αB crystallin protein. The invention provides methods for inhibiting protein aggregation in a mammalian subject by administering a polypeptide fragment of αB crystallin protein, e.g., a polypeptide of the invention. The invention also provides methods for screening for compositions that have chaperone activity or inhibit protein aggregation by screening polypeptide fragments of αB crystallin protein, e.g., a polypeptide of the invention.

In one aspect, the invention provides a polypeptide fragment of αB crystallin protein (and the nucleic acids encoding them) where one, some or all of the amino acids in the polypeptide fragment of αB crystallin protein comprise replacements with substituted amino acids. In one aspect, the invention provides methods to enhance the interaction of a polypeptide fragment of αB crystallin protein having molecular chaperone activity with unfolded proteins, denatured proteins, or native conformation proteins.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for polypeptide fragments of αB crystallin protein having molecular chaperone activity activity and for agents that can ameliorate disease, for example, protein aggregation disease, age-related myopathy, or cardiac ischemia.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of αB crystallin protein having molecular chaperone activity. A mimetic composition can also be within the scope of the invention if it stimulates a molecular chaperone activity in a cell or mammalian subject with a protein aggregation disease.

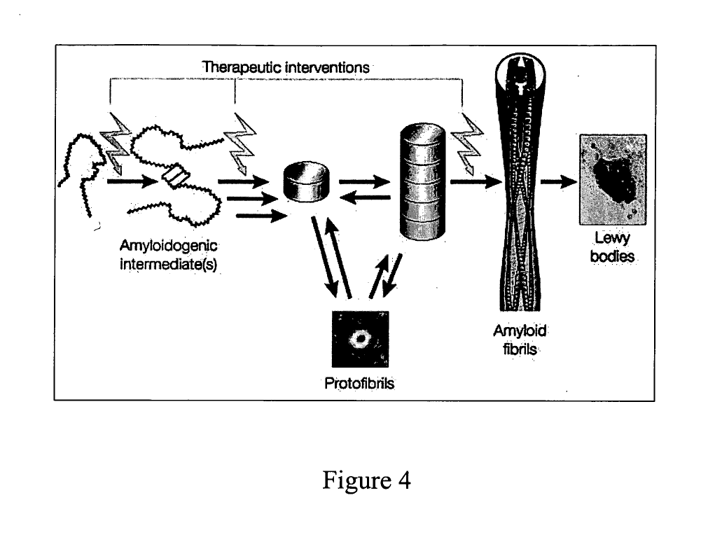

FIG. 4 shows a schematic diagram of amyloidosis in Alzheimer's disease (AD) and Parkinson's disease (PD) and possible interventions using Intellipeptides. Amyloid fibrils and plaques are hallmarks of AD neuropathology while Lewy bodies are characteristic of Parkinson's disease neuropathology. In AD, A-beta peptides that are amyloidogenic, aggregate to form A-beta plaques. Intellipeptides bind amyloid-beta, prevent aggregation and formation of cytotoxic amyloid plaques and prevent AD. In PD alpha-synuclein aggregates to form Lewy bodies. Intellipeptides bind alpha-synuclein, prevent aggregation and formation of Lewy bodies and prevent PD.

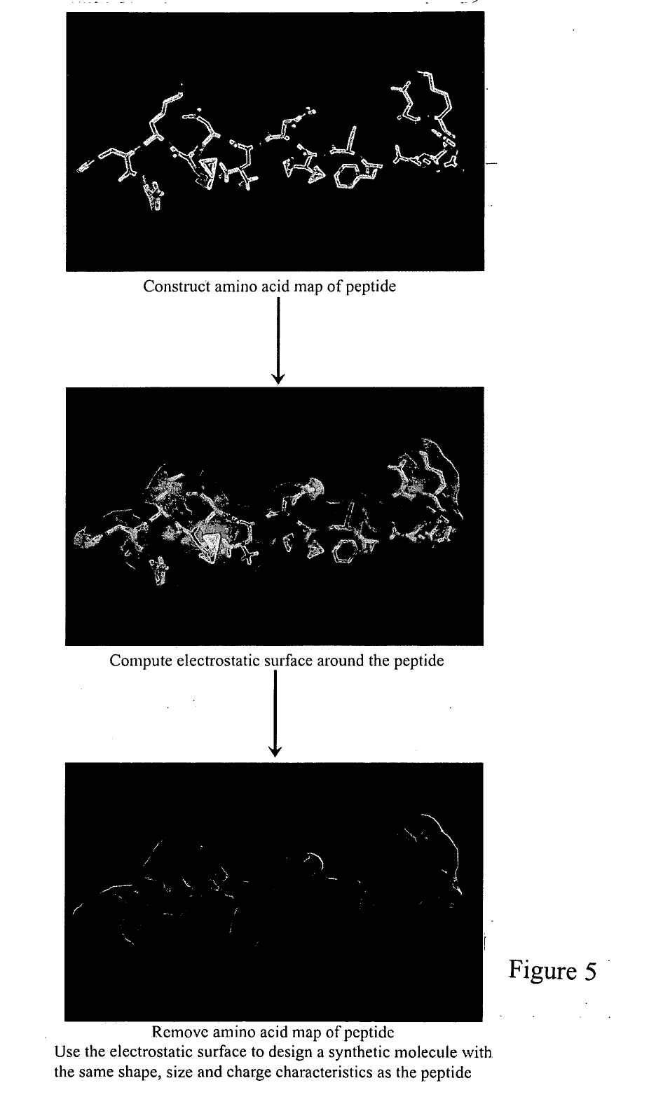

FIG. 5 shows a method for designing a polypeptide mimetic using a molecular model of an electrostatic surface to design a synthetic molecule with characteristics of a polypeptide. Using molecular modeling one can construct an amino acid map of the peptide of interest. From the amino acid map, one can compute an electrostatic surface around the peptide. By removing the amino acid map from the electrostatic surface map, one can use the electrostatic surface to design a synthetic molecule with the same shape, size and charge characteristics as a polypeptide.

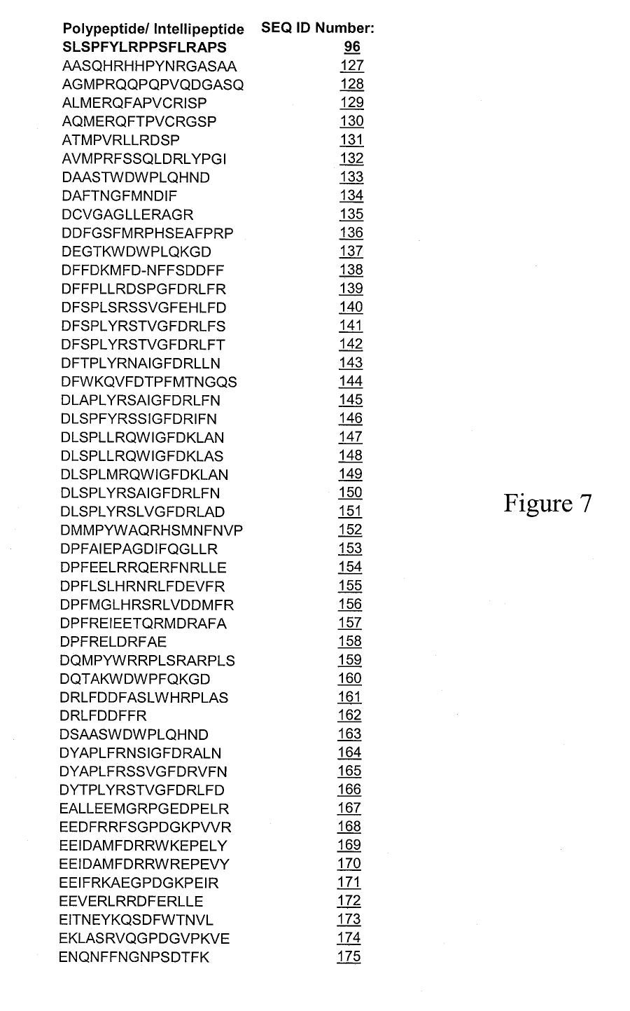

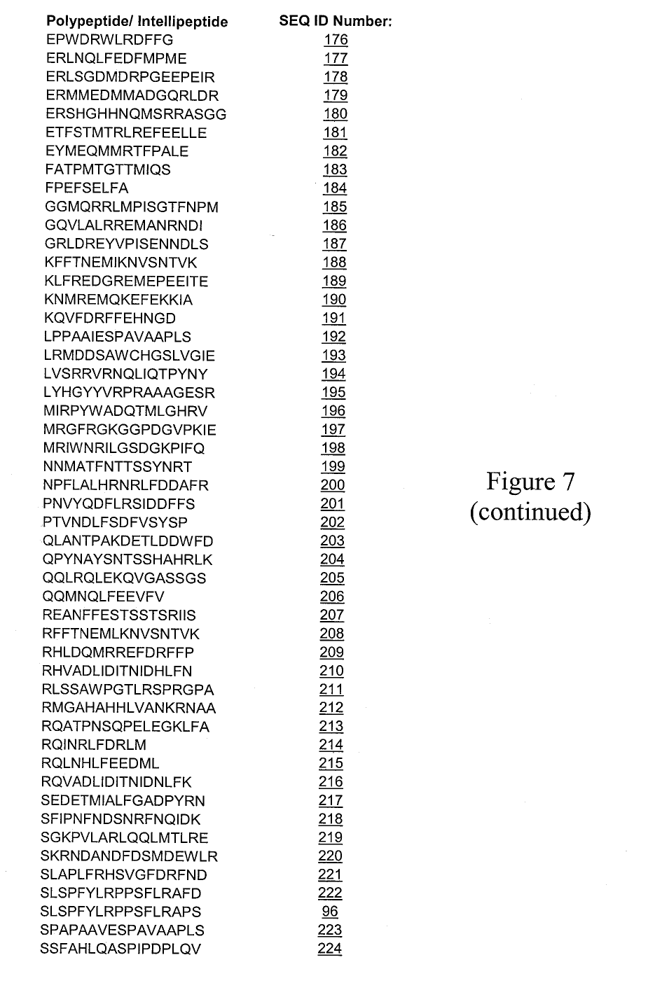

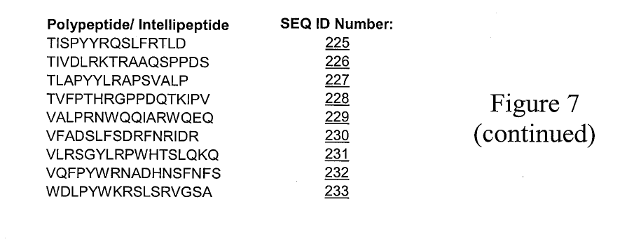

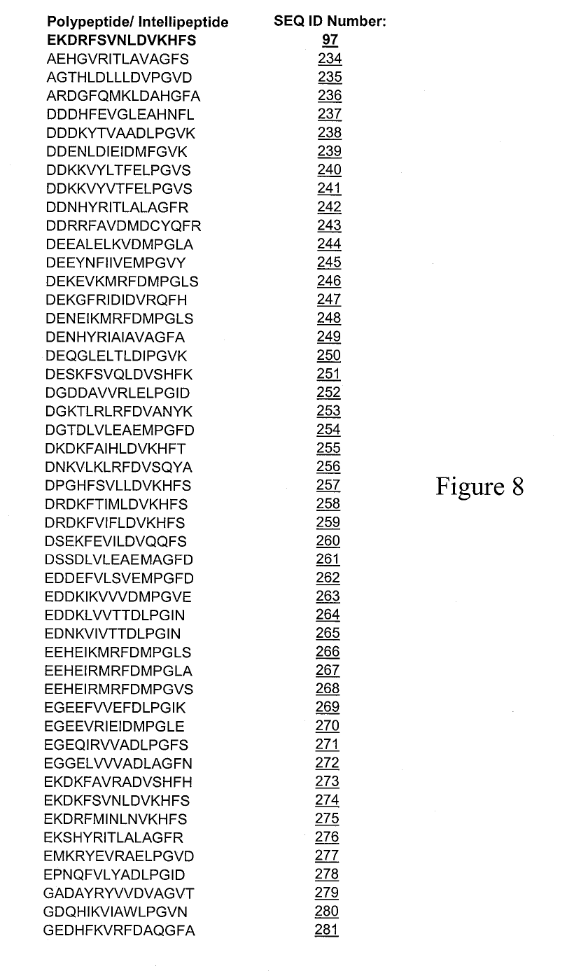

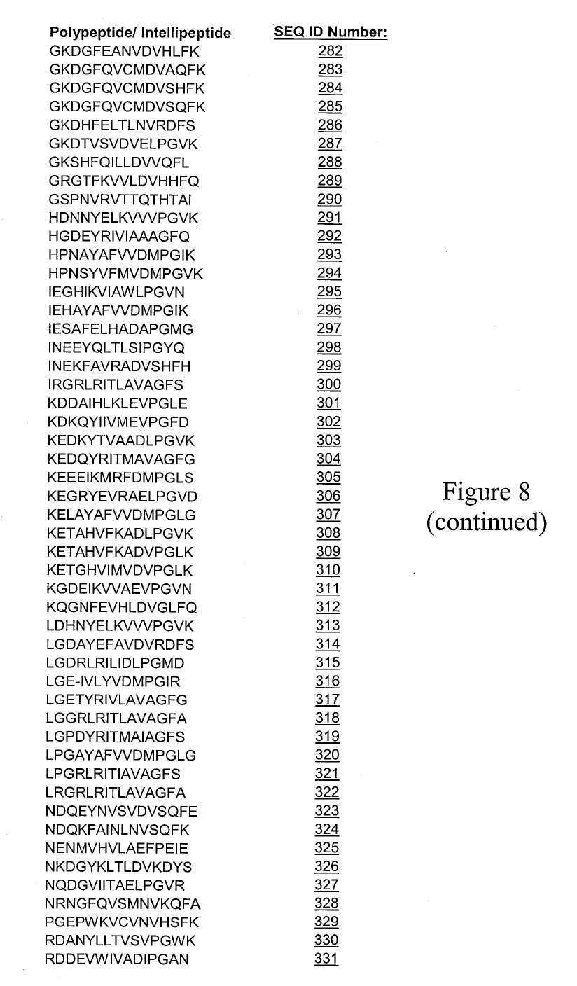

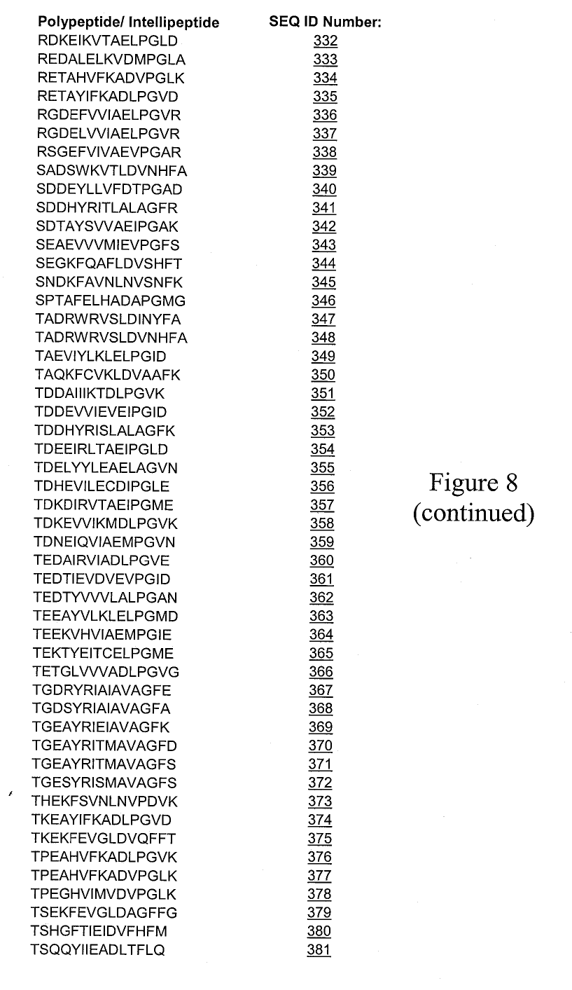

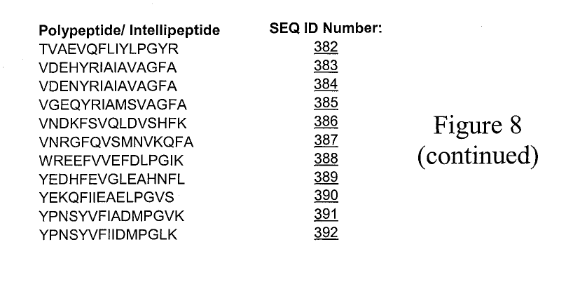

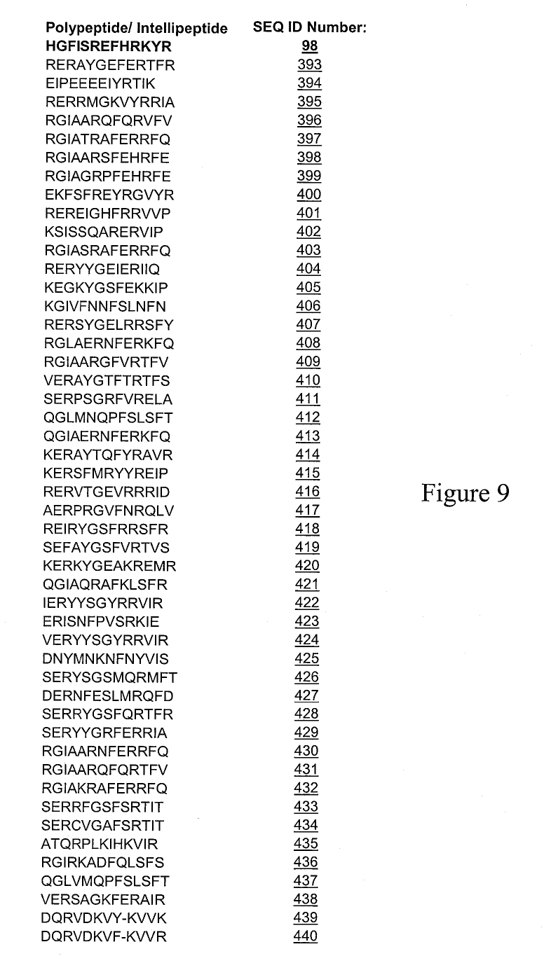

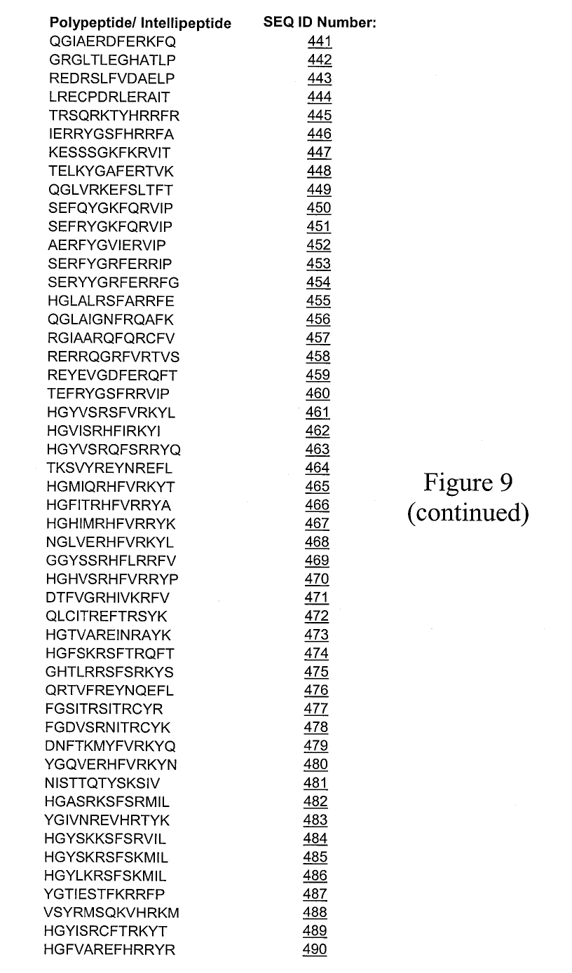

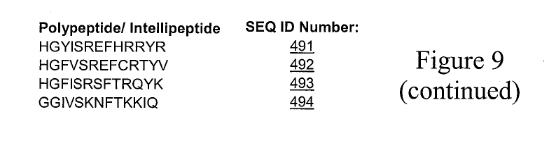

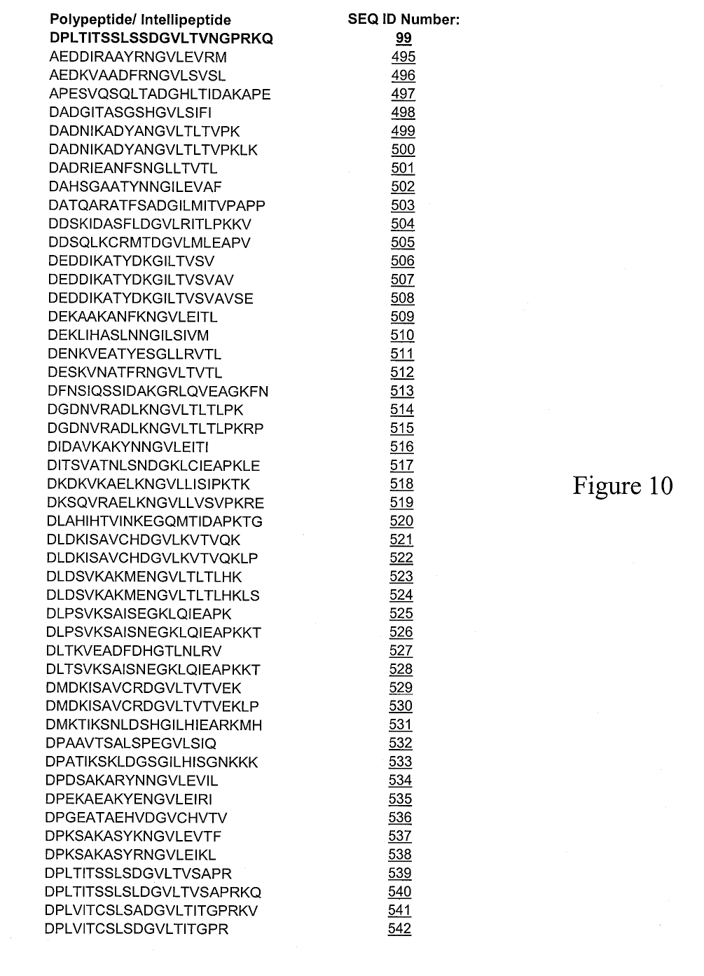

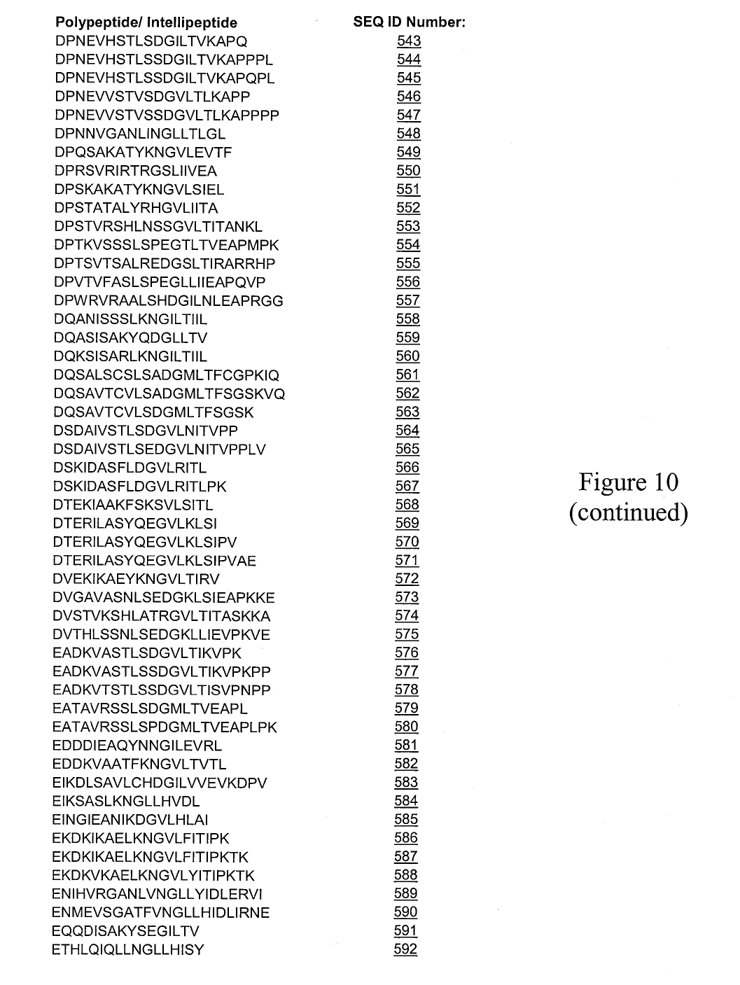

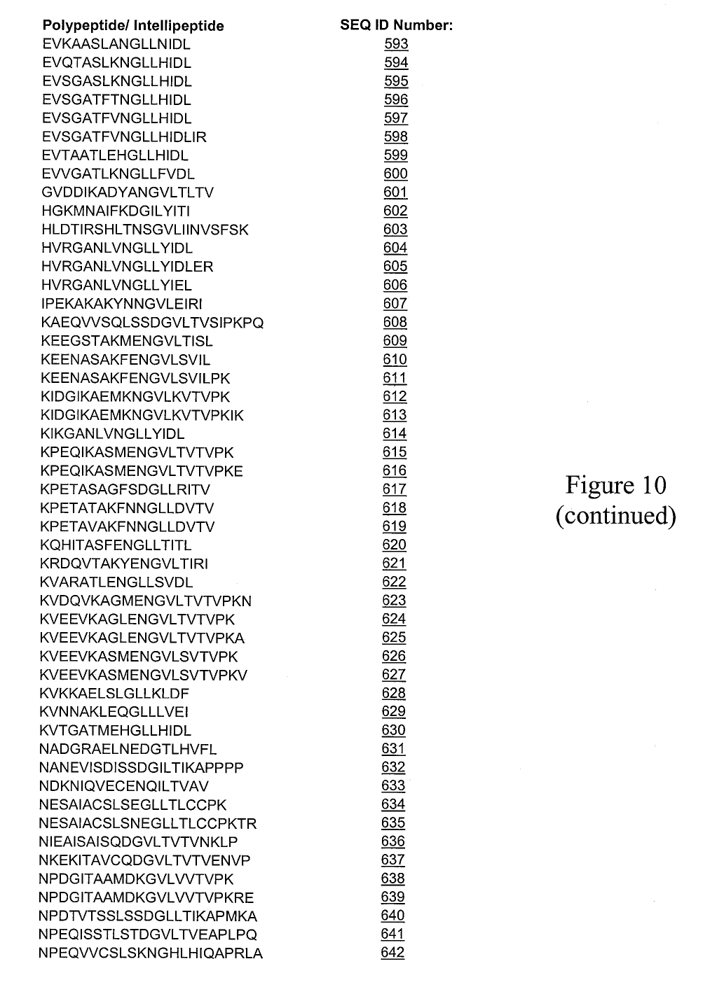

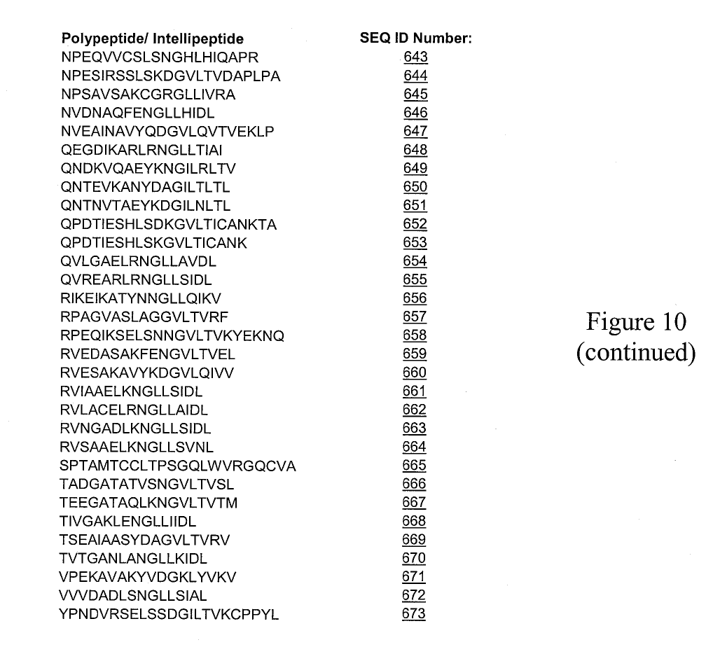

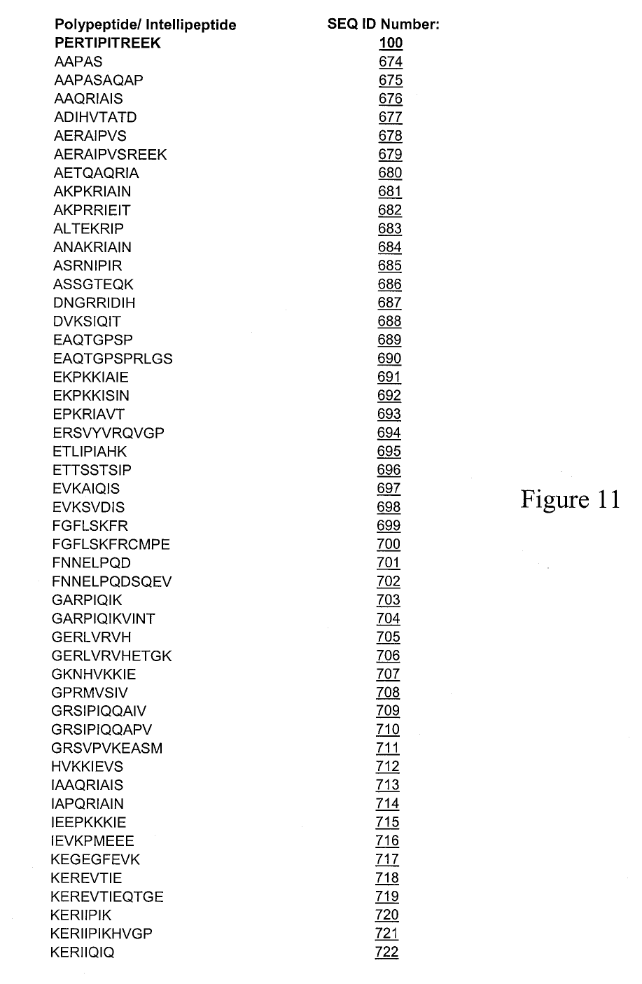

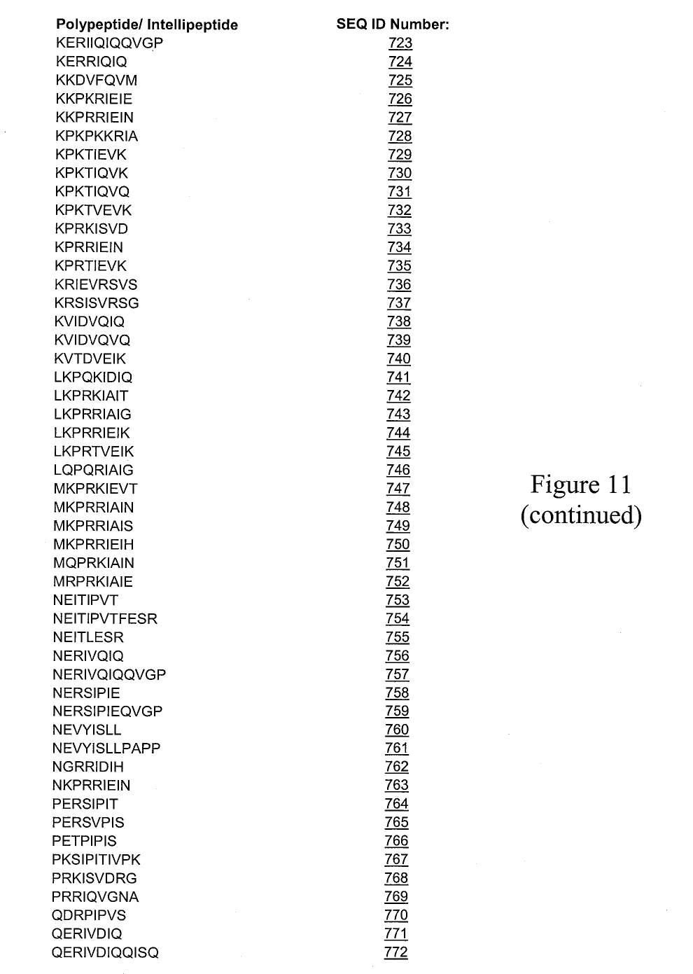

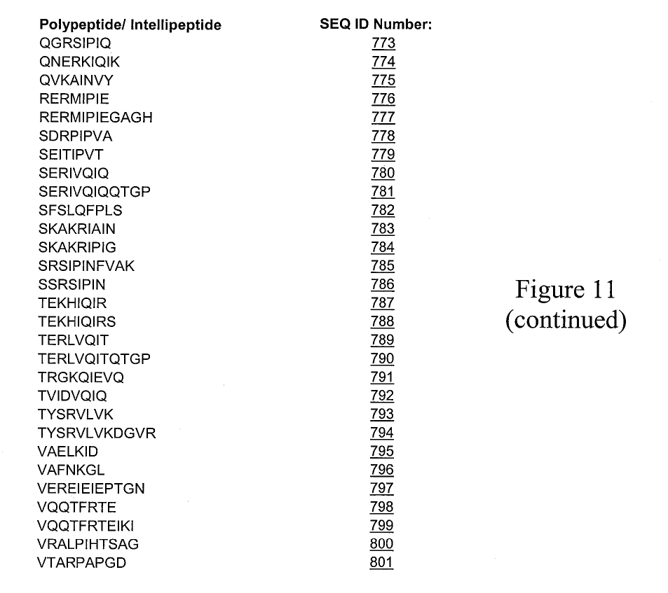

Intellipeptides or peptides that inhibit abnormal protein folding, protein unfolding, protein misfolding, or protein aggregation include, but are not limited to, Intellipeptides SLSPFYLRPPSFLRAPS, EKDRFSVNLDVKHFS, HGFISREFHRKYR, DPLTITSSLSSDGVLTVNGPRKQ, and PERTIPITREEK. FIG. 6 summarizes functional variants and peptide mimetics of Intellipeptides SLSPFYLRPPSFLRAPS, EKDRFSVNLDVKHFS, HGFISREFHRKYR, DPLTITSSLSSDGVLTVNGPRKQ, and PERTIPITREEK. FIG. 7 shows the amino acid sequence of a series of peptides derived from the Intellipeptide sequence SLSPFYLRPPSFLRAPS. FIG. 8 shows the amino acid sequence of a series of peptides derived from the Intellipeptide sequence EKDRFSVNLDVKHFS. FIG. 9 shows the amino acid sequence of a series of peptides derived from the Intellipeptide sequence HGFISREFHRKYR. FIG. 10 shows the amino acid sequence of a series of peptides derived from the Intellipeptide sequence DPLTITSSLSSDGVLTVNGPRKQ. FIG. 11 shows the amino acid sequence of a series of peptides derived from the Intellipeptide sequence PERTIPITREEK.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′-N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (prolyl)-acetic acid, or (prolyl)alkyl-acetic acid, where alkyl is defined above. Nitrile derivative (e.g., containing the CN-moiety in place of COOH) can be substituted for proline or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for □adioimmuno). One or more amino acids can be deleted, for example, from an αB crystallin polypeptide having molecular chaperone activity of the invention, resulting in modification of the structure of the polypeptide, without significantly altering its biological activity. For example, amino- or carboxyl-terminal, or internal, amino acids which are not required for molecular chaperone activity of αB crystallin protein can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Corp, Seattle, Wash.). The inclusion of a cleavable linker sequences such as Factor Xa or enterokinase (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

Intellipeptide analogs, polypeptide fragment of αB crystallin protein having molecular chaperone activity, are generally designed and produced by chemical modifications of a lead peptide, including, e.g., any of the particular peptides described herein, such as any of the following sequences: i) EKDRFSVNLDVKHFS; ii) DPLTITSSLSSDGVLTVNGPRKQ; iii) LTITSSLSDGVLTVNGPRK; iv) STSLSPFYLRPPSFLRAP; v) SLSPFYLRPPSFLRAPS; vi) GPERTIPITREEK; vii) PERTIPITREEK; viii) HGKHEERQDE; ix) HGFISREFHRKYR or functional variants or mimetics thereof. An exemplary polypeptide fragment of αB crystallin protein having molecular chaperone activity is presented; e.g., the N-terminal domain polypeptide fragment is ₉WIRRPFFPFHSP₂₀ or ₄₃SLSPFYLRPPSFLRAP₅₈, the α crystallin core domain polypeptide fragment is ₇₅FSVNLDVK₈₂ (β3), ₁₁₃FISREFHR₁₂₀, ₁₃₁LTITSSLS₁₃₈ (β8), ₁₄₁GVLTVNGP₁₄₈ (β9), ₇₃DRFSVNLDVKHFS₈₅, or ₁₃₁LTITSSLSDGV₁₄₁, or the C-terminal domain polypeptide fragment is ₁₅₇RTIPITRE₁₆₄, or functional variants thereof.

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Nat'l. Acad. Sci. USA 85: 2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res. 25: 3389-3402, 1977 and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.pov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89: 10915, 1989) alignments (B) of 50, expectation (E) of 10, M=5, N=−4, and a comparison of both strands.

“Polypeptide”, “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. “Polypeptide” and “protein” further refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

“Amino acid” or “functional variant or mimetic” of a polypeptide refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” or “variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, 1984).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules, 1980. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants”, as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

Functional variants of polypeptides of these genes and gene products are useful in the invention. “Functional variant” refers to a nucleic acid or protein having a nucleotide sequence or amino acid sequence, respectively, that is “identical,” “essentially identical,” “substantially identical,” “homologous” or “similar” (as described below) to a reference sequence which may, by way of non-limiting example, be the sequence of an isolated nucleic acid or protein, or a consensus sequence derived by comparison of two or more related nucleic acids or proteins, or a group of isoforms of a given nucleic acid or protein. Non-limiting examples of types of isoforms include isoforms of differing molecular weight that result from, e.g., alternate RNA splicing or proteolytic cleavage; and isoforms having different post-translational modifications, such as glycosylation; and the like.

Two sequences are said to be “identical” if the two sequences, when aligned with each other, are exactly the same with no gaps, substitutions, insertions or deletions.

Two sequences are said to be “essentially identical” if the following criteria are met. Two amino acid sequences are “essentially identical” if the two sequences, when aligned with each other, are exactly the same with no gaps, insertions or deletions, and the sequences have only conservative amino acid substitutions. Conservative amino acid substitutions are as described in Table 3.

TABLE 3
CONSERVATIVE AMINO ACID SUBSTITUTIONS

| Type of Amino | Groups of Amino Acids that Are Conservative |
|---|---|
| Acid Side Chain | Substitutions Relative to Each Other |
| Short side chain | Glycine, Alanine, Serine, Threonine and Methionine |
| Hydrophobic | Leucine, Isoleucine and Valine |
| Polar | Glutamine and Asparagine |
| Acidic | Glutamic Acid and Aspartic Acid |
| Basic | Arginine, Lysine and Histidine |
| Aromatic | Phenylalanine, Tryptophan and Tyrosine |

Two nucleotide sequences are “essentially identical” if they encode the identical or essentially identical amino acid sequence. As is known in the art, due to the nature of the genetic code, some amino acids are encoded by several different three base codons, and these codons may thus be substituted for each other without altering the amino acid at that position in an amino acid sequence. In the genetic code, TTA, TTG, CTT, CTC, CTA and CTG encode Leu; AGA, AGG, CGT, CGC, CGA and CGG encode Arg; GCT, GCC, GCA and GCG encode Ala; GGT, GGC, GGA and GGG encode Gly; ACT, ACC, ACA and ACG encode Thr; GTT, GTC, GTA and GTG encode Val; TCT, TCC, TCA and TCG encode Ser; CCT, CCC, CCA and CCG encode Pro; ATA, ATC and ATA encode Ile; GAA and GAG encode Glu; CAA and CAG encode Gln; GAT and GAC encode Asp; AAT and AAC encode Asn; AGT and AGC encode Ser; TAT and TAC encode Tyr; TGT and TGC encode Cys; AAA and AAG encode Lys; CAT and CAC encode His; TTT and TTC encode Phe, TGG encodes Trp; ATG encodes Met; and TGA, TAA and TAG are translation stop codons.

Two amino acid sequences are “substantially identical” if, when aligned, the two sequences are, (i) less than 30%, preferably <20%, more preferably <15%, most preferably <10%, of the identities of the amino acid residues vary between the two sequences; (ii) the number of gaps between or insertions in, deletions of and/or substitutions of, is <10%, more preferably <5%, more preferably <3%, most preferably <1%, of the number of amino acid residues that occur over the length of the shortest of two aligned sequences.

Two sequences are said to be “homologous” if any of the following criteria are met. The term “homolog” includes without limitation orthologs (homologs having genetic similarity as the result of sharing a common ancestor and encoding proteins that have the same function in different species) and paralog (similar to orthologs, yet gene and protein similarity is the result of a gene duplication).

One indication that nucleotide sequences are homologous is if two nucleic acid molecules hybridize to each other under stringent conditions. Stringent conditions are sequence dependent and will be different in different circumstances. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is about 0.02 M at pH 7 and the temperature is at least about 60° C.

Another way by which it can be determined if two sequences are homologous is by using an appropriate algorithm to determine if the above-described criteria for substantially identical sequences are met. Sequence comparisons between two (or more) polynucleotides or polypeptides are typically performed by algorithms such as, for example, the local homology algorithm of Smith and Waterman, Adv. Appl. Math. 2: 482, 1981; by the homology alignment algorithm of Needleman and Wunsch, J. Mol. Biol. 48: 443, 1970; by the search for similarity method of Pearson and Lipman, Proc. Natl. Acad. Sci. U.S.A. 85: 2444, 1988; and by computerized implementations of these algorithms (GAP, BESTFIT, FASTA, and TFASTA in the Wisconsin Genetics Software Package, version 10.2 Genetics Computer Group (GCG), 575 Science Dr., Madison, Wis.); BLASTP, BLASTN, and FASTA (Altschul et al., J. Mol. Biol. 215: 403-410, 1990); or by visual inspection.

Optimal alignments are found by inserting gaps to maximize the number of matches using the local homology algorithm of Smith and Waterman, Adv. Appl. Math. 2: 482-489, 1981. “Gap” uses the algorithm of Needleman and Wunsch, J. Mol. Biol. 48: 443-453, 1970) to find the alignment of two complete sequences that maximizes the number of matches and minimizes the number of gaps. In such algorithms, a “penalty” of about 3.0 to about 20 for each gap, and no penalty for end gaps, is used.

Homologous proteins also include members of clusters of orthologous groups of proteins (COGs), which are generated by phylogenetic classification of proteins encoded in complete genomes. To date, COGs have been delineated by comparing protein sequences encoded in 43 complete genomes, representing 30 major phylogenetic lineages. Each COG consists of individual proteins or groups of paralogs from at least 3 lineages and thus corresponds to an ancient conserved domain (see Tatusov et al., Science, 278: 631-637, 1997; Tatusov et al., Nucleic Acids Res. 29: 22-28, 2001; Chervitz et al., Science 282: 2022-2028, 1998; and http://www.ncbi.nlm.nih.gov/COG/).

The entirety of two sequences may be identical, essentially identical, substantially identical, or homologous to one another, or portions of such sequences may be identical or substantially identical with sequences of similar length in other sequences. In either case, such sequences are similar to each other. Typically, stretches of identical or essentially within similar sequences have a length of >12, preferably >24, more preferably >48, and most preferably >96 residues.

Polypeptides and Functional Variants Thereof

“Polypeptide” includes proteins, fusion proteins, oligopeptides and polypeptide derivatives, with the exception that peptidomimetics are considered to be small molecules herein. Although they are polypeptides, antibodies and their derivatives are described in a separate section. Antibodies and antibody derivatives are described in a separate section, but antibodies and antibody derivatives are, for purposes of the invention, treated as a subclass of the polypeptides and derivatives.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which may be a protein or a fusion protein. A proteolytic fragment may be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” is a polypeptide having a short amino acid sequence (i.e., 2 to about 200 amino acids). An oligopeptide is generally prepared by chemical synthesis.

Although oligopeptides and protein fragments may be otherwise prepared, it is possible to use recombinant DNA technology and/or in vitro biochemical manipulations. For example, a nucleic acid encoding an amino acid sequence may be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabelled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique. Merrifield, J. Am. Chem. Soc., 85: 2149, 1964; Stewart and Young, Solid Phase polypeptide Synthesis, Pierce Chemical Company, Rockford, Ill., 1984; Bodansky and Bodanszky, The Practice of polypeptide Synthesis, Springer-Verlag, New York, 1984; Atherton and Sheppard, Solid Phase polypeptide Synthesis: A Practical Approach, IRL Press, New York, 1989]. See, also, the specific method described in Example 1 below.

Alternatively, polypeptides of this invention may be prepared in recombinant systems using polynucleotide sequences encoding the polypeptides. For example, fusion proteins are typically prepared using recombinant DNA technology.

Functional Polypeptide Variant. A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wildtype” polypeptide. Functional variants of polypeptides can be prepared by chemical synthesis, including without limitation combinatorial synthesis.

Functional variants of polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare Functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

Chemically Modified Polypeptides. As contemplated by this invention, “polypeptide” includes those having one or more chemical modification relative to another polypeptide, i.e., chemically modified polypeptides. The polypeptide from which a chemically modified polypeptide is derived may be a wildtype protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

Polypeptides with N-Terminal or C-Terminal Chemical Groups. An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharma. Res. 10: 1268-1273, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

Polypeptides with a Terminal D-Amino Acid. The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Polypeptides with Substitution of Natural Amino Acids by Unnatural Amino Acids. Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Post-Translational Chemical Modifications. Different host cells will contain different post-translational modification mechanisms that may provide particular types of post-translational modification of a fusion protein if the amino acid sequences required for such modifications is present in the fusion protein. A large number (.about. 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e, N-formyl-methionine (fMet). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274: 22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the enzymes (such as, e.g., formylase) that catalyze such post-translational modifications will produce proteins having an amino terminal fMet residue (Guillon et al., J. Bacteriol. 174: 4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23: 263-267, 1998; and Driessen et al., CRC Crit. Rev. Biochem. 18: 281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50: 333-344, 1988, and Bradbury et al. Lung Cancer 14: 239-251, 1996. About 50% of known endocrine and neuroendocrine peptide hormones are alpha-amidated (Treston et al., Cell Growth Differ. 4: 911-920, 1993). In most cases, carboxy alpha-amidation is required to activate these peptide hormones.

Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683-687, 1994; Cohen and Shatzmiller, J. Mol. Graph., 11: 166-173, 1993; Wiley and Rich, Med. Res. Rev., 13: 327-384, 1993; Moore, Trends Pharmacol. Sci., 15: 124-129, 1994; Hruby, Biopolymers, 33: 1073-1082, 1993; Bugg et al., Sci. Am., 269: 92-98, 1993, all incorporated herein by reference].

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Peptides with a Reduced Isostere Pseudopeptide Bond. Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181-184, 1993, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

Peptides with a Retro-Inverso Pseudopeptide Bond. To confer resistance to proteolysis, peptide bonds may also be substituted by retro-inverso pseudopeptide bonds (Dalpozzo, et al., Int. J. Polypeptide Protein Res. 41: 561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid Derivatives. Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367-9371, 1992, and incorporated herein by reference). Peptoids are oligomers of N-substituted glycines. A number of N-alkyl groups have been described, each corresponding to the side chain of a natural amino acid.

Combinatorial Protein Design

The variants typically exhibit the same qualitative biological activity, however the chaperone activity may be altered from that of the original candidate variant protein, as needed. Alternatively, the variant may be designed such that the biological activity of the candidate variant protein is altered. For example, glycosylation sites may be altered or removed. Similarly, the biological function may be altered.

In addition, in some embodiments, it is desirable to have candidate variant proteins with altered chaperone activity that will bind to the target protein. Preferably, it would be desirable have proteins that exhibit oxidative stability, alkaline stability, and thermal stability.

A change in oxidative stability is evidenced by at least about 20%, more preferably at least about 50% increase of activity of a variant protein when exposed to various oxidizing conditions as compared to that of wild-type protein. Oxidative stability is measured by known procedures.

A change in alkaline stability is evidenced by at least about a 5% or greater increase or decrease (preferably increase) in the half life of the activity of a variant protein when exposed to increasing or decreasing pH conditions as compared to that of wild-type protein. Generally, alkaline stability is measured by known procedures.

A change in thermal stability is evidenced by at least about a 5% or greater increase or decrease (preferably increase) in the half-life of the activity of a variant protein when exposed to a relatively high temperature and neutral pH as compared to that of wild-type protein. Generally, thermal stability is measured by known procedures.

The candidate variant proteins and nucleic acids of the invention can be made in a number of ways. Individual nucleic acids and proteins can be made as known in the art and outlined below. Alternatively, libraries of candidate variant proteins can be made for testing.

In a preferred embodiment, the library of candidate variant proteins is generated from a probability distribution table. As outlined herein, there are a variety of methods of generating a probability distribution table, including using PDAT technology, sequence alignments, forcefield calculations such as self-consistent meant field (SCMF) calculations. In addition, the probability distribution can be used to generate information entropy scores for each position, as a measure of the mutational frequency observed in the library.

In this embodiment, the frequency of each amino acid residue at each variable position in the list is identified. Frequencies can be thresholded, wherein any variant frequency lower than a cutoff is set to zero. This cutoff is preferably about 1%, 2%, 5%, 10% or 20%, with about 10% being particularly preferred. These frequencies are then built into the library of candidate variant proteins. That is, as above, these variable positions are collected and all possible combinations are generated, but the amino acid residues that “fill” the library of candidate variant proteins are utilized on a frequency basis. Thus, in a non-frequency based library of candidate variant proteins, a variable position that has 5 possible residues will have about 20% of the proteins comprising that variable position with the first possible residue, 20% with the second, etc. However, in a frequency based library of candidate variant proteins, a variable position that has 5 possible residues with frequencies of about 10%, 15%, 25%, 30% and 20%, respectively, will have 10% of the proteins comprising that variable position with the first possible residue, 15% of the proteins with the second residue, 25% with the third, etc. As will be appreciated by those in the art, the actual frequency may depend on the method used to actually generate the proteins; for example, exact frequencies may be possible when the proteins are synthesized. However, when the frequency-based primer system outlined below is used, the actual frequencies at each position will vary, as outlined below.

As will be appreciated by those in the art and outlined herein, probability distribution tables can be generated in a variety of ways. In addition to the methods outlined herein, self-consistent mean field (SCMF) methods can be used in the direct generation of probability tables. SCMF is a deterministic computational method that uses a mean field description of rotamer interactions to calculate energies. A probability table generated in this way can be used to create libraries of candidate variant proteins as described herein. SCMF can be used in three ways: the frequencies of amino acids and rotamers for each amino acid are listed at each position; the probabilities are determined directly from SCMF (see Delarue et al. Pac. Symp. Biocomput. 109-21, 1997, expressly incorporated by reference). In addition, highly variable positions and non-variable positions can be identified. Alternatively, another method is used to determine what sequence is jumped to during a search of sequence space; SCMF is used to obtain an accurate energy for that sequence; this energy is then used to rank it and create a rank-ordered list of sequences (similar to a Monte Carlo sequence list). A probability table showing the frequencies of amino acids at each position can then be calculated from this list. Koehl et al., J. Mol. Biol. 239: 249, 1994; Koehl et al., Nat. Struc. Biol. 2: 163, 1995; Koehl et al., Curr. Opin. Struct. Biol. 6: 222, 1996; Koehl et al., J. Mol. Bio. 293: 1183, 1999; Koehl et al., J. Mol. Biol. 293: 1161, 1999; Lee, J. Mol. Biol. 236: 918, 1994; and Vasquez Biopolymers 36: 53-70, 1995; all of which are expressly incorporated by reference. Similar methods include, but are not limited to, OPLS-AA (Jorgensen, et al., J. Am. Chem. Soc., 118: 11225-11236, 1996; Jorgensen, W. L.; BOSS, Version 4.1; Yale University: New Haven, Conn., 1999); OPLS (Jorgensen, et al., J. Am. Chem. Soc., 110: 1657ff, 1988; Jorgensen, et al., J. Am. Chem. Soc. 112: 4768ff, 1990); UNRES (United Residue Forcefield; Liwo, et al., Protein Science, 2: 1697-1714, 1993; Liwo, et al., Protein Science, 2: 1715-1731, 1993; Liwo, et al., J. Comp. Chem. 18: 849-873, 1997; Liwo, et al., J. Comp. Chem., 18: 874-884, 1997; Liwo, et al., J. Comp. Chem. 19: 259-276, 1998; Forcefield for Protein Structure Prediction (Liwo, et al., Proc. Natl. Acad. Sci. USA, 96: 5482-5485, 1999); ECEPP/3 (Liwo et al., J Protein Chem 13(4): 375-80, 1994); AMBER 1.1 force field (Weiner, et al., J. Am. Chem. Soc. 106: 765-784, 1994); AMBER 3.0 force field (U. C. Singh et al., Proc. Natl. Acad. Sci. USA. 82: 755-759, 1994); CHARMM and CHARMM22 (Brooks, et al., J. Comp. Chem. 4: 187-217); cvff3.0 (Dauber-Osguthorpe, et al., Proteins: Structure, Function and Genetics, 4: 31-47, 1988); cff91 (Maple, et al., J. Comp. Chem. 15: 162-182, 1988); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.); all references hereby expressly incorporated by reference in their entirety.

In addition, a method of generating a probability distribution table is through the use of sequence alignment programs. In addition, the probability table can be obtained by a combination of sequence alignments and computational approaches. For example, one can add amino acids found in the alignment of homologous sequences to the result of the computation. Preferable one can add the wild type amino acid identity to the probability table if it is not found in the computation.

As will be appreciated, a library of candidate variant proteins created by recombining variable positions and/or residues at the variable position may not be in a rank-ordered list. In some embodiments, the entire list may just be made and tested. Alternatively, in a preferred embodiment, the secondary library is also in the form of a rank ordered list. This may be done for several reasons, including the size of the secondary library is still too big to generate experimentally, or for predictive purposes. This may be done in several ways. In one embodiment, the secondary library is ranked or filtered using the scoring functions of PDA™ to rank or filter the library members. Alternatively, statistical methods could be used. For example, the secondary library may be ranked or filtered by frequency score; that is, proteins containing the most of high frequency residues could be ranked higher, etc. This may be done by adding or multiplying the frequency at each variable position to generate a numerical score. Similarly, the secondary library different positions could be weighted and then the proteins scored; for example, those containing certain residues could be arbitrarily ranked or filtered.

In a one embodiment, the different protein members of the candidate variant library may be chemically synthesized. This is particularly useful when the designed proteins are short, preferably less than 150 amino acids in length, with less than 100 amino acids being preferred, and less than 50 amino acids being particularly preferred, although as is known in the art, longer proteins can be made chemically or enzymatically. See for example Wilken et al, Curr. Opin. Biotechnol. 9: 412-26, 1998, hereby expressly incorporated by reference.

In another embodiment, particularly for longer proteins or proteins for which large samples are desired, the candidate variant sequences are used to create nucleic acids such as DNA which encode the member sequences and which can then be cloned into host cells, expressed and assayed, if desired. Thus, nucleic acids, and particularly DNA, can be made which encodes each member protein sequence. This is done using well known procedures. The choice of codons, suitable expression vectors and suitable host cells will vary depending on a number of factors, and can be easily optimized as needed.

In a further embodiment, multiple PCR reactions with pooled oligonucleotides is done. In this embodiment, overlapping oligonucleotides are synthesized which correspond to the full length gene. Again, these oligonucleotides may represent all of the different amino acids at each variant position or subsets. These oligonucleotides can be pooled in equal proportions and multiple PCR reactions are performed to create full length sequences containing the combinations of mutations defined by the secondary library. In addition, this may be done using error-prone PCR methods. The different oligonucleotides can be added in relative amounts corresponding to the probability distribution table. The multiple PCR reactions thus result in full length sequences with the desired combinations of mutation in the desired proportions.

The total number of oligonucleotides needed is a function of the number of positions being mutated and the number of mutations being considered at these positions: (number of oligos for constant positions)+M1+M2+M3+ . . . Mn=(total number of oligos required), where Mn is the number of mutations considered at position n in the sequence.

In a further aspect, each overlapping oligonucleotide comprises only one position to be varied; in alternate embodiments, the variant positions are too close together to allow this and multiple variants per oligonucleotide are used to allow complete recombination of all the possibilities. That is, each oligo can contain the codon for a single position being mutated, or for more than one position being mutated. The multiple positions being mutated must be close in sequence to prevent the oligo length from being impractical. For multiple mutating positions on an oligonucleotide, particular combinations of mutations can be included or excluded in the library by including or excluding the oligonucleotide encoding that combination. For example, as discussed herein, there may be correlations between variable regions; that is, when position X is a certain residue, position Y must (or must not) be a particular residue. These sets of variable positions are sometimes referred to herein as a “cluster”. When the clusters are comprised of residues close together, and thus can reside on one oligonucleotide primer, the clusters can be set to the “good” correlations, and eliminate the bad combinations that may decrease the effectiveness of the library. However, if the residues of the cluster are far apart in sequence, and thus will reside on different oligonucleotides for synthesis, it may be desirable to either set the residues to the “good” correlation, or eliminate them as variable residues entirely. In an alternative embodiment, the library may be generated in several steps, so that the cluster mutations only appear together. This procedure, i.e., the procedure of identifying mutation clusters and either placing them on the same oligonucleotides or eliminating them from the library or library generation in several steps preserving clusters, can considerably enrich the experimental library with properly folded protein. Identification of clusters can be carried out by a number of ways, e.g. by using known pattern recognition methods, comparisons of frequencies of occurrence of mutations or by using energy analysis of the sequences to be experimentally generated (for example, if the energy of interaction is high, the positions are correlated). These correlations may be positional correlations (e.g. variable positions 1 and 2 always change together or never change together) or sequence correlations (e.g. if there is a residue A at position 1, there is always residue B at position 2). See: Pattern discovery in Biomolecular Data: Tools, Techniques, and Applications; edited by Jason T. L. Wang, Bruce A. Shapiro, Dennis Shasha. New York: Oxford University, 1999; Andrews, Harry C. Introduction to mathematical techniques in patter recognition; New York, Wiley-Interscience, 1972; Applications of Pattern Recognition; Editor, K. S. Fu. Boca Raton, Fla. CRC Press, 1982; Genetic Algorithms for Pattern Recognition; edited by Sankar K. Pal, Paul P. Wang. Boca Raton: CRC Press, c1996; Pandya, Abhijit S., Pattern recognition with Neural networks in C++/Abhijit S. Pandya, Robert B. Macy. Boca Raton, Fla.: CRC Press, 1996; Handbook of pattern recognition and computer vision/edited by C. H. Chen, L. F. Pau, P. S. P. Wang. 2nd ed. Signapore; River Edge, N.J.: World Scientific, c1999; Friedman, Introduction to Pattern Recognition: Statistical, Structural, Neural, and Fuzzy Logic Approaches; River Edge, N.J.: World Scientific, c1999, Series title: Serien a machine perception and artificial intelligence; vol. 32; all of which are expressly incorporated by reference. In addition programs used to search for consensus motifs can be used as well.

In addition, correlations and shuffling can be fixed or optimized by altering the design of the oligonucleotides; that is, by deciding where the oligonucleotides (primers) start and stop (e.g. where the sequences are “cut”). The start and stop sites of oligos can be set to maximize the number of clusters that appear in single oligonucleotides, thereby enriching the library with higher scoring sequences. Different oligonucleotides start and stop site options can be computationally modeled and ranked or filtered according to number of clusters that are represented on single oligos, or the percentage of the resulting sequences consistent with the predicted library of sequences.

The total number of oligonucleotides required increases when multiple mutable positions are encoded by a single oligonucleotide. The annealed regions are the ones that remain constant, i.e. have the sequence of the reference sequence.

Oligonucleotides with insertions or deletions of codons can be used to create a library expressing different length proteins. In particular computational sequence screening for insertions or deletions can result in secondary libraries defining different length proteins, which can be expressed by a library of pooled oligonucleotide of different lengths.

In a further aspect, the secondary library is done by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences can also be an artificial set; for example, from a probability table (for example generated using SCMF) or a Monte Carlo set. Similarly, the “family” can be the top 10 and the bottom 10 sequences, the top 100 sequences, etc. This may also be done using error-prone PCR.

Thus, in a further aspect, in silico shuffling is done using the computational methods described therein. That is, starting with either two libraries or two sequences, random recombinations of the sequences can be generated and evaluated.

Error-prone PCR can be done to generate the secondary library, See U.S. Pat. Nos. 5,605,793, 5,811,238, and 5,830,721, all of which are hereby incorporated by reference. This can be done on the optimal sequence or on top members of the library, or some other artificial set or family. In this embodiment, the gene for the optimal sequence found in the computational screen of the primary library can be synthesized. Error prone PCR is then performed on the optimal sequence gene in the presence of oligonucleotides that code for the mutations at the variant positions of the secondary library (bias oligonucleotides). The addition of the oligonucleotides will create a bias favoring the incorporation of the mutations in the secondary library. Alternatively, only oligonucleotides for certain mutations may be used to bias the library.

Gene shuffling with error prone PCR can be performed on the gene for the optimal sequence, in the presence of bias oligonucleotides, to create a DNA sequence library that reflects the proportion of the mutations found in the secondary library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked or filtered, some number of top scoring positions can be used to generate bias oligonucleotides; random positions may be chosen; a few top scoring and a few low scoring ones may be chosen; etc. What is important is to generate new sequences based on preferred variable positions and sequences.

PCR using a wild type gene or polypeptide sequence can be used. In this embodiment, a starting gene is used; generally, although this is not required, the gene is the wild type gene. In some cases it may be the gene encoding the global optimized sequence, or any other sequence of the list. In this embodiment, oligonucleotides are used that correspond to the variant positions and contain the different amino acids of the secondary library. PCR is done using PCR primers at the termini, as is known in the art. This provides two benefits; the first is that this generally requires fewer oligonucleotides and can result in fewer errors. In addition, it has experimental advantages in that if the wild type gene is used, it need not be synthesized. Ligation of PCR products can be done.

A variety of additional steps may be done to one or more candidate variant secondary libraries; for example, further computational processing can occur, candidate variant secondary libraries can be recombined, or cutoffs from different candidate variant secondary libraries can be combined. In a preferred embodiment, a candidate variant secondary library may be computationally remanipulated to form an additional secondary library (sometimes referred to herein as “tertiary libraries”). For example, any of the candidate variant secondary library sequences may be chosen for a second round of PDA™, by freezing or fixing some or all of the changed positions in the first secondary library. Alternatively, only changes seen in the last probability distribution table are allowed. Alternatively, the stringency of the probability table may be altered, either by increasing or decreasing the cutoff for inclusion. Similarly, the candidate variant secondary library may be recombined experimentally after the first round; for example, the best gene/genes from the first screen may be taken and gene assembly redone (for example, using techniques outlined below, multiple PCR, error prone PCR, or shuffling). Alternatively, the fragments from one or more good gene(s) to change probabilities at some positions. This biases the search to an area of sequence space found in the first round of computational and experimental screening.

Small Molecule Chemical Composition

“Small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 6(10): 991-1007, 2000, Bioanalytical applications of accelerator mass spectrometry for pharmaceutical research; and Enjalbal et al., Mass Spectrom Rev 19(3): 139-61, 2000, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

Methods of Use

Intellipeptides of the present invention may be useful in a variety of applications, including, but not limited to, therapeutic uses, e.g., to treat diseases and disorders associated with protein aggregation or misfolding, as well as in the manufacture and purification of polypeptides, including recombinantly-produced polypeptides. It is believed that the ability of a candidate therapeutic compound to prevent protein unfolding and aggregation in vitro may be correlated with the ability of the compound to inhibit protein unfolding and aggregation in vivo. In addition, it is believed that the ability of a candidate therapeutic compound to stabilize the functional structure of a protein in vitro may be correlated with the ability of the compound to assist that protein in performing its function in vivo.

The peptides presented here provide a versatile set of drug molecules that can be customized for use as therapeutic peptides to prevent protein aggregation or protein misfolding involved in disease. Examples of diseases related to protein aggregation or protein misfolding include, but are not limited to, amyloid-beta in Alzheimer's disease, beta/gamma crystallins and filaments in cataract, alpha-synuclein in Parkinson's, Huntingtin in Huntington's disease, rhodopsin in retinitis pigmentosa, prions, mad cow disease and others. Missense mutations leading to single amino acid changes in protein sequences have been linked to human disease. A majority of the approximately 16,0000 identified missense mutations affect folding or trafficking of proteins, rather than specifically affecting protein function. Disease linked missense mutations in integral membrane proteins result in membrane protein misassemby, for example, PMP-22 in Charcot-Marie Tooth disease, aquaporin in diabetes insipidis, vasopressin receptor in diabetes insipidis, rhodopsin in retinitis pigmentosa, connexin 32 in Charcot-Marie Tooth disease, CFTR in cystic fibrosis.

Further the peptides can be used for the stabilization of therapeutic proteins such as vaccines, insulin, growth factors, monoclonal and antibodies. The Intellipeptides can be used in the purification of proteins to aid with folding of the proteins to their functional 3D conformation.

Accordingly, the present invention describes a variety of methods related to the use of Intellipeptides. In one embodiment, the present invention provides a method of inhibiting protein unfolding or reducing protein aggregation by providing an Intellipeptide to a cell or solution comprising said protein. In a related embodiment, the present invention includes a method of restoring correct or proper protein folding, by providing an Intellipeptide to a cell or solution comprising said protein. In addition, the present invention further provides a method of enhancing the production and/or isolation of a recombinantly-produced polypeptide, by providing an Intellipeptide to a cell or solution comprising said polypeptide.

Intellipeptides may be provided to a cell or solution by a variety of means available in the art. For example, synthesized Intellipeptides may be directly provided to a solution or into a cell. In addition, Intellipeptides may be provided to a cell or solution by introducing an expression vector comprising a polynucleotide sequence encoding an Intellipeptide with regulatory elements that drive expression of said Intellipeptide in a cell. The polynucleotide sequence may further comprise additional coding regions, including, e.g., a secretion signal such that the Intellipeptide will be secreted from the cell and/or additional elements regulating expression of the encoded Intellipeptide, of which a large variety are known and available in the art, including those used for inducible expression of peptides and polypeptides. Thus, the present invention further includes polynucleotide sequences encoding Intellipeptides and expression vectors comprising the same, including, e.g., viral vectors.

Intellipeptides can be used as therapeutics for, but not limited to, protein aggregation diseases, including, e.g., Alzheimer's disease, Cataract, Parkinson's, Huntington's, Lou Gehrig's, Bovine Spongiform Encephalopathy (Mad Cow's disease), Prion disease, Macular Degeneration and Retinitis Pigmentosa. In addition, Intellipeptides can stabilize proteins and/or peptides used as therapeutics including but not limited to vaccines, insulin, growth factors and monoclonal antibodies. Intellipeptides help fold and stabilize the 3-dimensional structure of proteins during purification. Thus, the present invention provides a method for treating a disease or disorder comprising the administration of an Intellipeptide to a patient in need thereof. A diagram related to the therapeutic applications of Intellipeptides in provided in FIG. 1.

In one embodiment, the invention provides a method for treatment of Alzheimer's disease by providing an Intellipeptide to a patient with Alzheimer's. Alzheimer's disease (AD) is a devastating neurodegenerative condition characterized by loss of short-term memory, disorientation, and impairment of judgment and reasoning. AD is the most common dementia in elderly population and is estimated to affect more than twenty-five million people worldwide in some degree. A hallmark event in AD is the deposition of insoluble protein aggregates, known as amyloid, in brain parenchyma and cerebral vessel walls. The main component of amyloid is a 4.3 KDa hydrophobic peptide, named amyloid beta-peptide (Amyloid-beta.) that is encoded on the chromosome 21 as part of a much longer precursor protein (APP) (Selkoe, Science 275: 630-631, 1997). Genetic, biochemical, and neuropathological evidence accumulated in the last 10 years strongly suggest that amyloid plays an important role in early pathogenesis of AD and perhaps triggers the disease (Selkoe, 1997). In the case of Alzheimer's disease, the aggregation of the proteins amyloid-beta and tau is associated with degeneration of nerve cells and nerve processes. Amyloid-beta aggregation leads to the formation of toxic plaque which causes nerve cell death. Tau on the other hand plays a key role in the structure of nerve processes. Tau associates with a cytoskeletal filament called tubulin to form microtubules that are the core of nerve processes. Post-translational modifications, specifically hyper-phosphorylation of tau leads to destabilization of the tau-tubulin interaction. Destabilization of microtubules leads to degeneration of nerve processes. Eventually the tau-tubulin interaction is so severely destabilized that tau dissociates from tubulin and becomes free. Unbound tau has a tendency to aggregate and form neurofibrillary tangles. Neurofibrillary tangles are neurotoxic and lead to neurodegeneration. Amyloid-beta plaques and neurofibrillary tangles are the hallmarks of Alzheimer's disease.

In another embodiment, the present invention provides a method for the treatment of diabetes by providing an Intellipeptide to a patient with diabetes. In a particular embodiment, an Intellipeptide is provided to the patient as a stabilizing molecule for the oral and or nasal delivery of insulin for diabetics. Accordingly, the present invention includes a method of treating diabetes, comprising administering insulin in combination with an Intellipeptide to a patient in need thereof.

Even though, oral and nasal delivery methods for insulin are convenient and pain-free and have been recently approved for use in humans by the FDA, currently the most widely used method for delivering insulin to Type I diabetics is through injections. Many diabetics need multiple injections in a single day which can be both painful and inconvenient. Insulin, a peptide drug, is unstable at room temperature, cannot be absorbed through the gastrointestinal (GI) membrane and is prone to proteolysis by the enzymes of the GI tract. Molecules that can stabilize insulin by protecting it from harsh environmental conditions including temperate, pH and proteolytic enzymes will enable the delivery of insulin via an oral or nasal route. Though small molecules and polymers exist that will protect insulin from pH and proteolytic enzymes, no existing polymer or small molecule can maintain the structural integrity of insulin for long periods of time. Intellipeptides can bind to insulin and stabilize its structure, maintain its function and protect it from temperate, pH and proteolytic enzymes. Formulations of insulin with one or more Intellipeptides will enable insulin delivery via routes including but not limited to oral, nasal, device and patch methods.

In another embodiment, the present invention includes a method of manufacturing and/or purification of a peptide or protein by introduction of an Intellipeptide to a cell or solution comprising said peptide or protein. Modern pharmaceutical discovery processes increasingly focus on developing drugs against specific molecular targets and have greatly increased the requirements within the industry for the production of recombinant proteins. Expression of high quality proteins is essential for drug discovery and drug therapeutics. As the number of potential drug targets and protein and peptide drugs increases the requirement for recombinant proteins is likely to increase. Development of robust methods to produce target proteins in a soluble form and in significant amounts is an essential requirement for modern drug discovery. Intellipeptides can serve as folding aids to fold proteins that are synthesized using biotechnological methods such as bacterial or mammalian expression systems.

Intellipeptides are a versatile set of molecules that target one or more intermediates in the protein misfolding and aggregation disease pathway. In one embodiment, Intellipeptides are designed to bind and stabilize non-native intermediates of conformationally compromised proteins, for example, Intellipeptides bind and stabilize amyloid-beta and prevent its aggregation.

### EXEMPLARY EMBODIMENTS

### Example 1

Identification of αB Crystallin Peptides that Bind Beta-Amyloid

αB Crystallin peptides that bind the target protein beta-amyloid were identified by screening an αB crystallin protein pin array, which provides a systematic method for evaluating interactions between peptides corresponding to residues 1-175 of human αB crystallin and selected target proteins in a parallel and simultaneous manner.

Peptides were synthesized on derivatized polyethylene pins arranged in a microtiter plate format. Each peptide was 8 amino acids in length and consecutive peptides were offset by 2 amino acids. All peptides were covalently bonded to the surface plastic pins. The first immobilized peptide was ₁MDIAIHHP₈ and the last peptide was ₁₆₈PAVTAAPK₁₇₅ for the human αB crystallin.

After synthesis, the pins were pre-coated at room temperature with 2% Bovine Serum Albumin (BSA), 0.1% Tween-20 and 0.1% Sodium azide in 10 mM phosphate buffered saline pH 7.2 (PBS) for 1 hr, and washed three times for 10 mins each with 10 mM PBS. To screen for binding to the peptides, fixed concentrations of the target protein beta amyloid was dissolved in 10 mM PBS, containing 0.05% Tween-20, added to each well and incubated for 1 hr at room temperature. The pin array was washed three times for 10 mins each with 10 mM PBS, containing 0.05% Tween-20. Monoclonal or polyclonal primary antibodies for the target protein beta amyloid was diluted into PBS buffer, added to each well and incubated for 1 hr at room temperature. Subsequently, the pin array was washed three times for 10 mins each with 10 mM PBS containing 0.05% Tween-20. Anti-rabbit horse-radish peroxidase conjugate secondary antibodies was diluted into PBS buffer, added to each well and incubated for 1 hr at room temperature. The pin array was washed three times for 10 mins each with 10 mM PBS containing 0.05% Tween-20. A chromogenic substrate of horse-radish peroxidase 3,3′,5,5′-Tetramethylbenzidine (TMB) (Pharmingen, San Diego, Calif.) which is colorless was added, and the reaction was carried out for 45 min.

Pins displaying a positive reaction resulted in the formation of a blue color. The reaction was stopped by adding 6N sulfuric acid, which changes the color from blue to yellow. The absorption at 450 nm was measured by an ELISA reader (BioTek, Winooski, Vt.).

In all, 84 peptides corresponding to entire primary sequence of human αB crystallin were screened for binding to the target protein beta amyloid (Table 2). 36/84 peptides bound the target protein beta amyloid.

The pin array was regenerated by sonication in a water bath (VWR Aquasonic, West Chester, Pa.) with 100 mM PBS, containing 1% Sodium dodecyl sulfate (SDS) and 0.1% 2-Mercaptoethanol@60° C. for 10 mins. Next, the pin array was rinsed three times in deionized water, preheated to 60° C. for 30 secs each time, and shaken in water preheated at 60° C. for 30 min. The pin array was then washed with methanol at 60° C. for 15 secs and air-dried and stored @−20° C. for future use. Each target protein was assayed 2-5 times against the human αB crystallin protein pin array peptides to verify the reproducibility of the results. Peptides numbered 5, 7, 8, 9, 13, 19, 22, 23, 24, 27, 35, 38, 45, 51, 52, 56, 57, 61, 66, 71, 72, and 79 in Table 2 reproducibly tested positive in the protein pin assay.

TABLE 2
List of sequential 8-mer peptides of human αB crystallin that were
synthesized in a protein pin array format. Columns 2 and 6 list the
peptide sequence. Columns 3 and 7 list the hydrophobicity as
provided by manufacturer. Columns 4 and 8 list the
calculated molecular weight as provided by the
manufacturer (Mimotopes, San Diego, CA).

| # | Peptide | Hydrophobicity | Mol. Wt. |
|---|---|---|---|
| 1 | MDIAIHHP | 0.67 | 915.09 |
| 2 | IAIHHPWI | 1.12 | 968.17 |
| 3 | IHHPWIRR | 0.6 | 1096.31 |
| 4 | HPWIRRPF | 0.67 | 1090.31 |
| 5 | WIRRPFFP | 0.88 | 1100.35 |
| 6 | RRPFFPFH | 0.62 | 1085.3 |
| 7 | PFFPFHSP | 0.95 | 957.12 |
| 8 | FPFHSPSR | 0.51 | 956.09 |
| 9 | FHSPSRLF | 0.63 | 972.13 |
| 10 | SPSRLFDQ | 0.27 | 931.03 |
| 11 | SRLFDQFF | 0.63 | 1041.19 |
| 12 | LFDQFFGE | 0.68 | 984.09 |
| 13 | DQFFGEHL | 0.47 | 974.05 |
| 14 | FFGEHLLE | 0.73 | 973.11 |
| 15 | GEHLLESD | 0.18 | 880.92 |
| 16 | HLLESDLF | 0.7 | 955.09 |
| 17 | LESDLFPT | 0.59 | 903.02 |
| 18 | SDLFPTST | 0.49 | 848.93 |
| 19 | LFPTSTSL | 0.79 | 847 |
| 20 | PTSTSLSP | 0.44 | 770.86 |
| 21 | STSLSPFY | 0.66 | 882.99 |
| 22 | SLSPFYLR | 0.72 | 964.15 |
| 23 | SPFYLRPP | 0.7 | 958.15 |
| 24 | FYLRPPSF | 0.83 | 1008.21 |
| 25 | LRPPSFLR | 0.57 | 967.2 |
| 26 | PPSFLRAP | 0.61 | 866.05 |
| 27 | SFLRAPSW | 0.71 | 945.1 |
| 28 | LRAPSWFD | 0.62 | 973.11 |
| 29 | APSWFDTG | 0.56 | 861.92 |
| 30 | SWFDTGLS | 0.64 | 893.96 |
| 31 | FDTGLSEM | 0.44 | 880.99 |
| 32 | TGLSEMRL | 0.4 | 888.07 |
| 33 | LSEMRLEK | 0.16 | 987.2 |
| 34 | EMRLEKDR | −0.27 | 1058.24 |
| 35 | RLEKDRFS | −0.12 | 1032.18 |
| 36 | EKDRFSVN | −0.13 | 976.06 |
| 37 | DRFSVNLD | 0.19 | 947.02 |
| 38 | FSVNLDVK | 0.44 | 903.04 |
| 39 | VNLDVKHF | 0.46 | 953.1 |
| 40 | LDVKHFSP | 0.47 | 924.07 |
| 41 | VKHFSPEE | 0.19 | 954.06 |
| 42 | HESPEELK | 0.25 | 968.09 |
| 43 | SPEELKVK | 0.04 | 911.07 |
| 44 | EELKVKVL | 0.32 | 939.16 |
| 45 | LKVKVLGD | 0.39 | 853.06 |
| 46 | VKVLGDVI | 0.68 | 824.02 |
| 47 | VLGDVIEV | 0.72 | 824.97 |
| 48 | GDVIEVHG | 0.37 | 806.87 |
| 49 | VIEVHGKH | 0.36 | 900.04 |
| 50 | EVHGKHEE | −0.18 | 945.99 |
| 51 | HGKHEERQ | −0.41 | 1002.06 |
| 52 | KHEERQDE | −0.6 | 1052.08 |
| 53 | EERQDEHG | −0.47 | 980.96 |
| 54 | RQDEHGFI | 0.14 | 983.06 |
| 55 | DEHGFISR | 0.16 | 942.01 |
| 56 | HGFISREF | 0.48 | 974.1 |
| 57 | FISREFHR | 0.35 | 1073.24 |
| 58 | SREFHRKY | −0.1 | 1104.25 |
| 59 | EFHRKYRI | 0.13 | 1130.33 |
| 60 | HRKYRIPA | 0.11 | 1022.23 |
| 61 | KYRIPADV | 0.28 | 943.12 |
| 62 | RIPADVDP | 0.28 | 863.98 |
| 63 | PADVDPLT | 0.42 | 808.9 |
| 64 | DVDPLTIT | 0.55 | 854.97 |
| 65 | DPLTITSS | 0.49 | 814.91 |
| 66 | LTITSSLS | 0.7 | 802.94 |
| 67 | ITSSLSSD | 0.35 | 790.84 |
| 68 | SSLSSDGV | 0.25 | 732.75 |
| 69 | LSSDGVLT | 0.5 | 772.86 |
| 70 | SDGVLTVN | 0.37 | 785.85 |
| 71 | GVLTVNGP | 0.57 | 737.85 |
| 72 | LTVNGPRK | 0.16 | 866.03 |
| 73 | VNGPRKQV | 0.04 | 879.02 |
| 74 | GPRKQVSG | −0.04 | 809.92 |
| 75 | RKQVSGPE | −0.12 | 881.99 |
| 76 | QVSGPERT | 0.04 | 854.93 |
| 77 | SGPERTIP | 0.23 | 837.95 |
| 78 | PERTIPIT | 0.49 | 908.09 |
| 79 | RTIPITRE | 0.27 | 967.16 |
| 80 | IPITREEK | 0.16 | 967.15 |
| 81 | ITREEKPA | −0.02 | 925.07 |
| 82 | REEKPAVT | −0.1 | 911.04 |
| 83 | EKPAVTAA | 0.19 | 767.89 |
| 84 | PAVTAAPK | 0.36 | 735.89 |

### Example 2

Effect of Intellipeptides on pH-Induced Aggregation of Amyloidβ In Vitro

In vitro assays were performed to measure the ability of the human αB crystallin derived peptides identified in Example 1 to inhibit aggregation of pH-induced amyloid beta (1-42) aggregation. Amyloid-beta (1-42), the peptide implicated in Alzheimer's disease, aggregates under acidic conditions (pH 5.2). Aggregation assay were performed using routine procedures, in the presence or absence of a synthesized peptide identified in Example 1, at the acidic pH of 5.2. Aggregation was measured spectrophotometrically as light scattering at a wavelength of 360 nm. Aggregated amyloid-beta in the absence of other peptides was assigned a value of 100% aggregation and used to normalize aggregation in the presence of synthesized peptides.

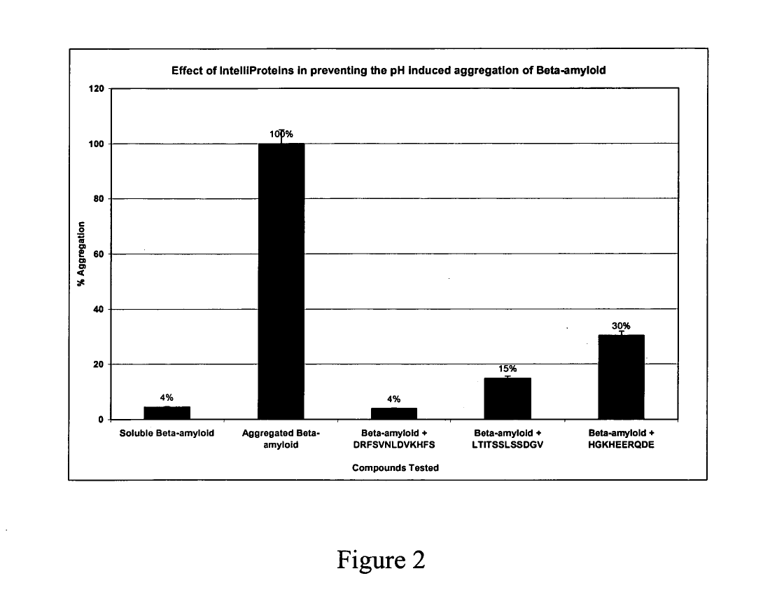

Three peptides identified containing sequences identified using protein pin arrays and tested for their efficacy in preventing the pH induced aggregation of amyloid-beta were able to reproducibly prevent the aggregation of amyloid-beta in vitro. The sequences of these peptides are DRFSVNLDVKHFS or LTITSSLSDGV or HGKHEERQDE, respectively. FIG. 2 provides a graph depicting the effect of each peptide on the aggregation of beta-amyloid aggregation. DRFSVNLDVKHFS (4% aggregation) was most effective in preventing the pH induced aggregation of amyloid-beta at pH 5.2. LTITSSLSDGV (15% aggregation) (bar4) and HGKHEERQDE (30% aggregation) (bar5) prevented the pH induced aggregation of amyloid-beta at pH 5.2. These data demonstrated that peptides identified by screening an αB crystallin protein pin array have the ability to reduce protein amyloid-beta aggregation.

FIG. 2 shows a bar graph depicting the effect of Intellipeptides on the pH induced aggregation of amyloid-beta in vitro. Aggregation was plotted as a bar graph with the standard error for duplicate experiments. The 1st vertical bar is the scattering measured for soluble amyloid-beta at pH 5.2. The 2nd vertical bar represents aggregated amyloid-beta at pH 5.2 in the absence of any other peptide or protein (100% aggregation). The 3rd-5th vertical bars represent amyloid-beta at pH 5.2 in the presence of either DRFSVNLDVKHFS or LTITSSLSDGV or HGKHEERQDE respectively. DRFSVNLDVKHFS is most effective in preventing the pH induced aggregation of amyloid-beta (4% aggregation) at pH 5.2. LTITSSLSDGV (15% aggregation)(bar4) and HGKHEERQDE (30% aggregation) (bar5) inhibit the pH induced aggregation of amyloid-beta at pH 5.2.

### Example 3

Effect of Intellipeptides on Cu⁺⁺⁺-Induced Aggregation of Amyloid β In Vitro

In vitro assays were also performed to measure the ability of the human αB crystallin derived peptides identified in Example 1 to inhibit aggregation of Cu⁺⁺⁺-induced amyloid beta (1-42) aggregation. Aggregation assays were performed using routine procedures, in the presence or absence of a synthesized peptide identified in Example 1, in the presence of 100 mM Cu⁺⁺⁺. Aggregation was measured spectrophotometrically as light scattering at a wavelength of 360 nm. Aggregated amyloid-beta was assigned a value of 100% aggregation and used to normalize aggregation in the presence of synthesize peptides.

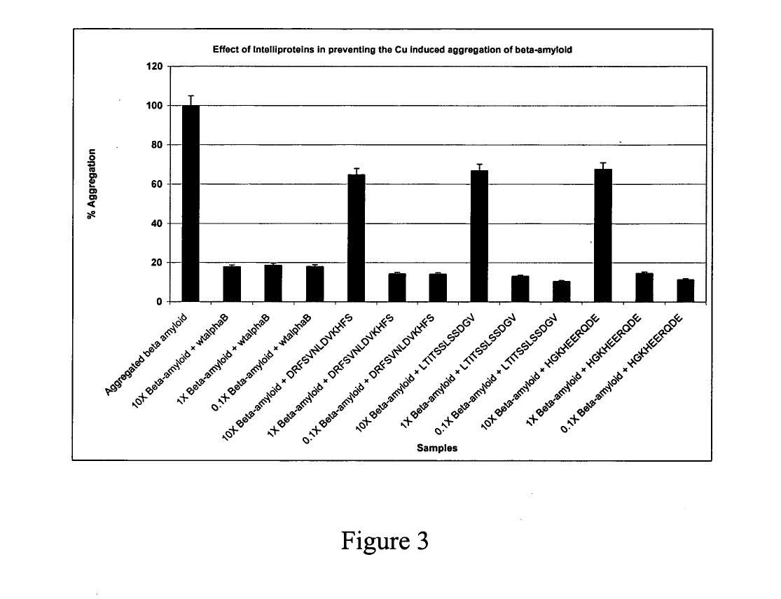

Three peptides containing sequences identified using protein pin arrays and tested for their efficacy in preventing the Cu⁺⁺⁺-induced aggregation of amyloid-beta were able to reproducibly prevent the aggregation of amyloid-beta in vitro, as shown in FIG. 3. The peptides DRFSVNLDVKHFS, LTITSSLSDGV, HGKHEERQDE were equally effective in preventing the Cu⁺⁺⁺-induced aggregation of amyloid-beta at 1:1 and 1:10 molar ratio of amyloid-beta to wt αB crystallin or its peptides. The synthetic peptides, DRFSVNLDVKHFS, LTITSSLSDGV, and HGKHEERQDE were less effective in preventing the Cu⁺⁺⁺ induced aggregation of amyloid-beta at 10:1 molar ratio of amyloid-beta to either wt αB crystallin or its peptides. These data demonstrated that peptides identified by screening an αB crystallin protein pin array have the ability to reduce protein amyloid-beta aggregation, strongly supporting their use in the therapeutic treatment of Alzheimer's disease.

FIG. 3 shows a bar graph depicting the effect of Intellipeptides on the Cu⁺⁺⁺ induced aggregation of amyloid-beta in vitro. Aggregation was measured spectrophotometrically as light scattering at a wavelength of 360 nm. Aggregated amyloid-beta was assigned a value of 100% aggregation and used to normalize aggregation in the presence of synthesize peptides. Aggregation was plotted as a bar graph with the standard error for duplicate experiments. The 1 st vertical bar is the scattering measured for aggregated amyloid-beta (100% aggregation) in the presence of 100 mM Cu⁺⁺⁺. The 2nd-4th vertical bars is the scattering from amyloid-beta in the presence of wild type recombinant human αB crystallin. The 5th-13th vertical bars are the scattering of amyloid-beta in the presence of the synthetic peptides, DRFSVNLDVKHFS or LTITSSLSDGV or HGKHEERQDE at selected concentrations. A prefix of 10× represents a 10:1 molar concentration of amyloid-beta to either wild type αB crystallin or its peptides. A prefix of 1× represents a 1:1 molar concentration of amyloid-beta to either wt αB crystallin or its peptides. A prefix of 0.1× represents a 1:10 molar concentration of amyloid-beta to either wt αB crystallin or its peptides.

### Example 4

Generation of Intellipeptide Library

The sequences of the peptides identified in Example 2 and 3 for the ability to decrease protein aggregation were used to generate a library of related Intellipeptides, based upon molecular modeling of human αB crystallin.

The homology modeling program Molecular Operating Environment (Chemical Computing Group, Inc, Montreal, Canada) was used to construct a 3D homology model of human αB crystallin using the wheat sHSP 16.9 crystal structure as the template. MOE employs a number of techniques including and not limited to multiple sequence alignments, structure superposition, contact analyzer and fold identification to develop homology models based on available high resolution crystal and/or NMR structures of the template protein molecule. In building the homology model of human αB crystallin, the primary sequence of human αB crystallin was first coarsely aligned to that of the template protein, wheat sHSP 16.9 primary sequence using ClustalX. The predicted secondary structure of human αB crystallin was then obtained (JPred) and verified with the available spin labeling information about the structural elements. The human αB crystallin 3-dimensional structure was evaluated using Procheck and the ModelEval module of MOE.

Percent surface hydrophobicities of the peptides sequences were calculated using an electrostatic potential to the 3D structure of human αB crystallin.

The primary sequence and the predicted secondary structure of human αB crystallin holoprotein were aligned with the primary sequence and the predicted secondary structure of all the proteins in the small heat shock protein family whose sequence is known. Homologous peptides that are structurally conserved and have primary sequence similar to the human αB crystallin peptides were selected. A library of peptides having sequences related to and derived from i) EKDRFSVNLDVKHFS or ii) LTITSSLSDGVLTVNGPRK was prepared. The amino acid sequence of selected peptides is presented in FIGS. 5 and 6.

### Example 5

Interactive Domains for Chaperone Activity in the Small Heat Shock Protein, Human αB Crystallin

Protein pin arrays identified seven interactive sequences for chaperone activity in human αB crystallin using natural lens proteins, β_{H} crystallin and γD crystallin, and in vitro chaperone target proteins, alcohol dehydrogenase and citrate synthase. The N-terminal domain contained two interactive sequences, ₉WIRRPFFPFHSP₂₀ and ₄₃SLSPFYLRPPSFLRAP₅₈. The α crystallin core domain contained four interactive sequences, ₇₅FSVNLDVK₈₂ (β3), ₁₁₃FISREFHR₁₂₀, ₁₃₁LTITSSLS₁₃₈ (β8) and ₁₄₁GVLTVNGP₁₄₈ (β9). The C-terminal domain contained one interactive sequence, ₁₅₇RTIPITRE₁₆₄ that included the highly conserved I-X-I/V amino acid motif. Two interactive sequences, ₇₃DRFSVNLDVKHFS₈₅ and ₁₃₁LTITSSLSDGV₁₄₁ belonging to the α crystallin core domain were synthesized as peptides and assayed for chaperone activity in vitro. Both synthesized peptides inhibited the thermal aggregation of β_{H} crystallin, alcohol dehydrogenase and citrate synthase in vitro. Five of the seven chaperone sequences identified by the pin arrays overlapped with sequences identified previously as sequences for subunit-subunit interactions in human αB crystallin. The results suggested that interactive sequences in human αB crystallin have dual roles in subunit-subunit assembly and chaperone activity.

Human αB crystallin is a small heat shock protein (sHSP) and molecular chaperone. sHSPs are characterized by molecular weights <43 kDa, low sequence similarity, up-regulation in response to environmental stress and an ability to protect against the unfolding and aggregation of proteins through activity as molecular chaperones. Ingolia and Craig, Proc Natl Acad Sci USA 79: 2360-4,1982; Klemenz et al., Proc Natl Acad Sci USA 88: 3652-6, 1991; Merck et al., J Biol Chem 268: 1046-52, 1993; de Jong et al., Mol Biol Evol 10: 103-26, 1993; Groenen et al., Eur J Biochem 225: 1-19, 1994; de Jong et al., Int J Biol Macromol 22: 151-62, 1998; Bloemendal et al., Prog Biophys Mol Biol 86: 407-85, 2004. sHSPs are ubiquitous in cells and tissues throughout the plant and animal kingdoms and are upregulated in age-related myopathies, cardiac ischemia, and a variety of protein aggregation diseases including Alexander's disease, Alzheimer's disease, Creutzfeld-Jakob disease and Parkinson's disease. Iwaki et al., Cell 57: 71-8, 1989; Iwaki et al., Am J Pathol 140: 345-56, 1992; Kato et al., Acta Neuropathol (Berl) 84: 443-8, 1992; Shinohara et al., J Neurol Sci 119: 203-8, 1993; Goebel and Bornemann, Virchows Arch B Cell Pathol Incl Mol Pathol 64: 127-35, 1993; van Noort et al., Nature 375: 798-801, 1995; Jackson et al., Neuropathol Appl Neurobiol 21: 18-26, 1995; Lobrinus et al., Neuromuscul Disord 8: 77-86, 1998; Renkawek et al., Neuroreport 10: 2273-6, 1999; van Rijk and Bloemendal, Ophthalmologica 214: 7-12, 2000; Yeboah and White, Croat Med J 42: 523-6, 2001. In the lens where α crystallins comprise approximately 33% of the total protein content, the accumulation of post-translational modifications is associated with protein unfolding that favors attractive interactions between proteins and formation of aggregates large enough to result in light scattering and cataract. Garland et al., Basic Life Sci 49: 347-52, 1988; Harding, Lens Eye Toxic Res 8: 245-50, 1991; Miesbauer et al., J Biol Chem 269: 12494-502, 1994; Goode and Crabbe, Comput Chem 19: 65-74, 1995; Lund et al., Exp Eye Res 63: 661-72, 1996; Hanson et al., Exp Eye Res 67: 301-12, 1998; Yan and Hui, Yan Ke Xue Bao 16: 91-6, 2000; Feng et al., J Biol Chem 275: 11585-90, 2000; Garner et al., Biochim Biophys Acta 1476: 265-78, 2000; Lapko et al., Protein Sci 10: 1130-6, 2001; Kim et al., Biochemistry 41: 14076-84, 2002; Ueda et al., Invest Ophthalmol Vis Sci 43: 205-15, 2002. In theory, high concentrations of a crystallins in lens cytoplasm can bind unfolding β/γ crystallin proteins, stabilize transparent cell structure and protect against aggregation and opacification through their function as molecular chaperones. Fu and Liang, Invest Ophthalmol Vis Sci 44: 1155-9, 2003; Srivastava and Srivastava, Mol Vis 9: 110-8, 2003; del Valle et al., Biochim Biophys Acta 1601: 100-9, 2002; Slingsby and Clout, Eye 13 Pt 3b′: 395-402, 1999; Hook and Harding, Int J Biol Macromol 22: 295-306, 1998; Takemoto and Boyle, Int J Biol Macromol 22: 331-7, 1998. In a normal lens, αB crystallin is a structural protein, that interacts weakly with the β/γ crystallins and is closely associated with the filament network. Fu and Liang, J Biol Chem 277: 4255-60, 2002; Nicholl and Quinlan, Embo J 13: 945-53, 1994.

While X-ray crystal structures exist for α and γ crystallin, the structure of αB crystallin is based on spectroscopic data and homology models. Kim et al., Nature 394: 595-9, 1998; Basak et al., J Mol Biol 328: 1137-47, 2003; Purkiss et al., J Biol Chem 277: 4199-205, 2002; Sergeev et al., Mol Vis 4: 9, 1998; Hejtmancik et al., Protein Eng 10: 1347-52, 1997; Norledge et al., Protein Sci 6: 1612-20, 1997; van Montfort et al., Nat Struct Biol 8: 1025-30, 2001; Carver and Lindner, Int J Biol Macromol 22: 197-209, 1998; Ghosh and Clark, Protein Sci 14: 684-95, 2005. Spectroscopic data, secondary structure prediction and X-ray crystal structures of two homologous sHSPs, Methanococcus jannaschi (Mj) HSP 16.5 and wheat sHSP16.9, indicated that sHSPs are characterized by three structural domains, an N-terminal domain that varies in primary sequence, an α crystallin core domain that is conserved in primary sequence and secondary structure and a C-terminal extension that is variable in sequence. In the crystal structures of Mj sHSP 16.5 and wheat sHSP16.9, the N-terminal domain is largely helical or unstructured, the α crystallin core domain is an immunoglobulin-like fold and the C-terminal extension domain protrudes from the α crystallin core domain and is unstructured and flexible. Carver and Lindner, Int J Biol Macromol 22:197-209, 1998. The immunoglobulin-like fold adopted by the α crystallin core domain is a β sandwich composed of two anti-parallel β sheets formed by six to nine β strands connected by loops of variable lengths. The formation of dimers in wheat sHSP16.9 is due to interactions between the β2 and β3 strands of one monomer with the β6 strand contained in the loop connecting β5 and β7 of another monomer. The C-terminal extension contains a conserved I-X-I/V amino acid motif where I is Isoleucine, V is Valine and X is any natural amino acid. In wheat sHSP 16.9, the I-X-I motif of one monomer interacts with residues of the β4 and β8 strands of another monomer to form the higher order dodecameric quaternary structure observed in the crystal structure. While αB crystallin contains the same three structural domains found in Mj sHSP16.5 and wheat sHSP16.9, the complex assembly of human αB crystallin is larger, and more polydisperse than the two sHSPs that have been crystallized. This suggests that the dimer interface and the oligomerization interface in αB crystallin may be different from the smaller homologous sHSPs. Proteolysis and domain swapped chimeric mutants of αB crystallin and Caenorhabditis elegans (C. elegans) sHSP12.2 indicated that sequences in all three structural domains of sHSPs were important for complex assembly and chaperone activity. Kokke et al., Cell Stress Chaperones 6: 360-7, 2001; Saha and Das, Proteins 57: 610-7, 2004. The identity of specific residues or sequences in each domain important for the complex assembly and chaperone activity of αB crystallin and other small heat shock proteins remain to be determined.

In a previous study using a protein pin array of sequential and overlapping eight-mer peptides of human αB crystallin, the interactive domains required for subunit-subunit interactions and complex assembly in human αB crystallin were identified. Ghosh and Clark, Protein Sci 14: 684-95, 2005. The N-terminal sequence, ₃₇LFPTSTSLSPFYLRPPSF₅₄, three α crystallin core domain sequences, ₇₅FSVNLDVK₈₂ (β3), ₁₃₁LTITSSLS₁₃₈ (β8) and ₁₄₁GVLTVNGP₁₄₈ (β9) and the C-terminal sequence, ₁₅₅PERTIPITREEK₁₆₆ were identified as subunit-subunit interaction sites in αB crystallin. The pin array studies confirmed and expanded on spectroscopic observations, mutational studies, proteolytic degradation experiments and a two-hybrid screen that characterized interactive domains in sHSPs. The subunit-subunit interactive domains identified by the pin arrays were consistent with the dimer and complex interfaces (β3, β8, β9 and the I-X-I/V amino acid motif) identified in the crystal structures of Mj sHSP 16.5 and wheat sHSP 16.9 with one exception. The pin arrays did not identify sequences in the loop region connecting β5 and β7 as interactive sequences for dimerization in αB crystalline. Kim et al., Nature 394: 595-9, 1998; van Montfort et al., Nat Struct Biol 8: 1025-30, 2001; Ghosh and Clark, Protein Sci 14: 684-95, 2005. In separate reports using peptide scanning techniques, sequences for subunit-subunit assembly and chaperone function were identified in αB crystallin and a small heat shock protein, sHSPB, that were consistent with the sequences identified by the pin arrays. Ghosh and Clark, Protein Sci 14: 684-95, 2005; Sreelakshmi et al., Biochemistry 43: 15785-95, 2004; Lentze and Narberhaus, Biochem Biophys Res Commun 325: 401-7, 2004. A sequence in the α crystallin core domain of αA crystallin, KFVIFLDVKHFSPEDLTVK which is homologous to the sequence ₇₅FSVNLDVK₈₂ from the α crystallin core domain of human αB crystallin was reported to have chaperone-like activity in vitro. Sharma et al., J Biol Chem 275: 3767-71, 2000.

In this report, protein pin arrays identified and characterized interactive sequences that were mapped to a 3-D structural model. Seven interactive domains for chaperone function in human αB crystallin were identified as sequences that interacted with denatured β_{H} crystallin, γD crystallin, alcohol dehydrogenase and citrate synthase. Two of the interactive peptides, ₇₃DRFSVNLDVKHFS₈₅ and ₁₃₁LTITSSLSDGV₁₄₁, were synthesized and observed to have chaperone activity in vitro against the thermal aggregation of β_{H} crystallin, ADH and CS. The seven interactive sequences for chaperone function identified by the pin arrays overlapped with the interactive domains for subunit-subunit interactions and complex assembly identified previously. Ghosh and Clark, Protein Sci 14: 684-95, 2005. Taken together, these data suggest that interactive domains in αB crystallin mediate dual functions of chaperone activity and subunit-subunit assembly.

### Example 6

Materials and Methods

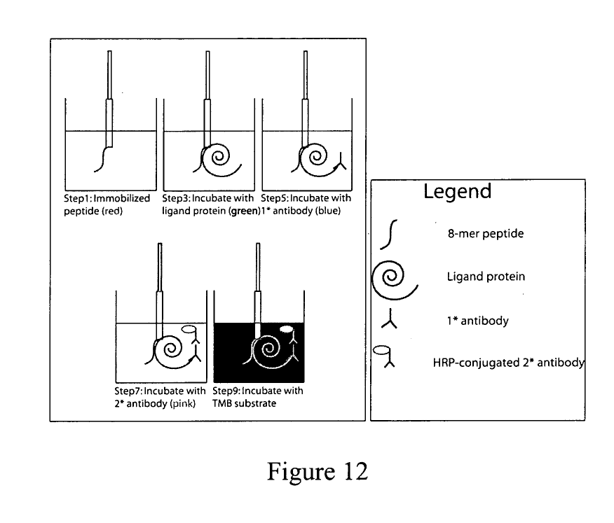

Synthesis of Pins, binding and detection of peptides binding to ligand proteins. The αB crystallin protein pin array was used to measure the interaction between peptides and chaperone target proteins including bovine β_{H} crystallin, human γD crystallin, equine alcohol dehydrogenase (ADH) and porcine citrate synthase (CS) as described (FIG. 12). Ghosh and Clark, Protein Sci 14: 684-95, 2005.

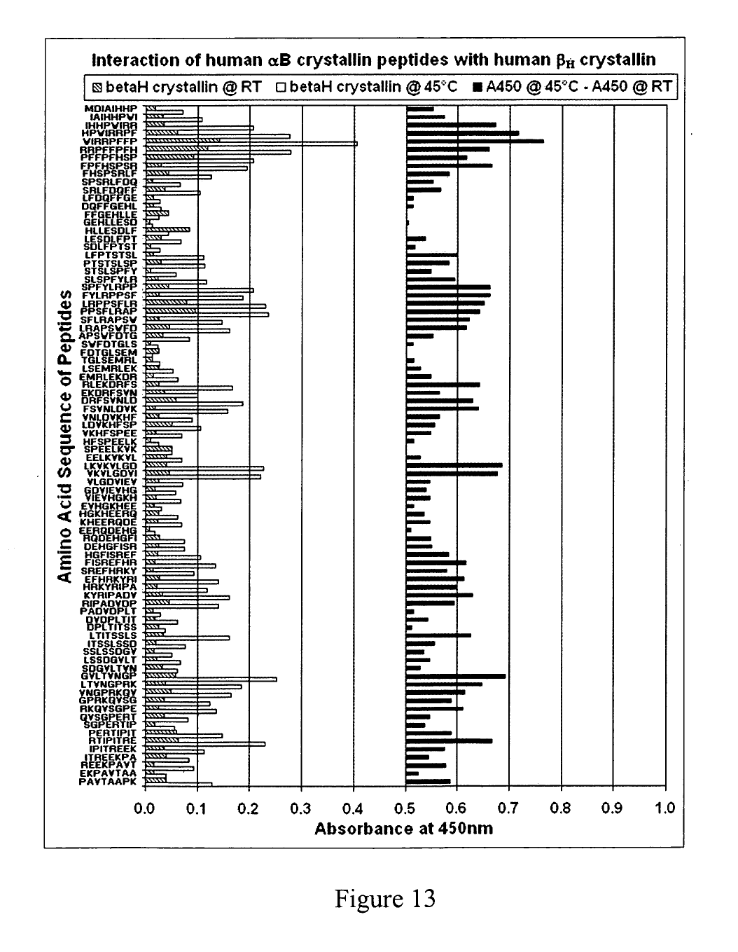

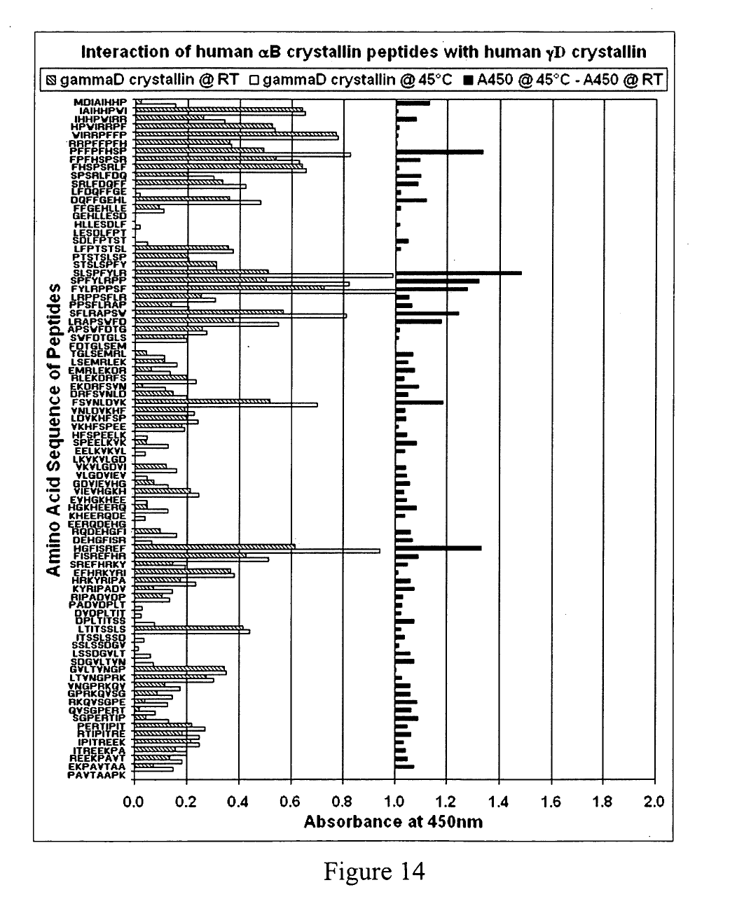

FIG. 12 shows a schematic for the protein pin array assay. Refer to the methods section for detailed protocols. The absorbance corresponding to the blue coloration in each well was measured at λ=450 nm and plotted against the amino acid sequence of the corresponding peptide in that well (FIGS. 13 and 14). Wells containing peptides that have strong interactions with target proteins are dark blue and wells containing peptides that have weak or no interaction with target proteins are light blue or clear.

The purity of the target proteins used in the pin array assays, bovine β_{H} crystallin, human γD crystallin, equine alcohol dehydrogenase (ADH) and porcine citrate synthase (CS) were determined to be >90% by SDS-PAGE. In addition, primary antibodies for each target protein were specific to that target protein and consequently contaminating proteins that may bind to the peptides will not be detected. Eighty-four sequential and overlapping peptide fragments corresponding to residues 1-175 of human αB crystallin were synthesized employing a simultaneous peptide synthesis strategy developed by Geysen, called Multipin Peptide Synthesis (Mimotopes, San Diego, Calif.). Geysen, Southeast Asian J Trop Med Public Health 21: 523-33, 1990; Chin et al., J Org Chem 62: 538-539, 1997. Peptides were immobilized on derivatized polyethylene pins arranged in a microtiter ELISA plate format. Each peptide was eight amino acids in length and consecutive peptides were offset by two amino acids. All peptides were bound covalently to the surface of the plastic pins. The first peptide was ₁MDIAIHHP₈ and the last peptide was ₁₆₈PAVTAAPK₁₇₅ for human αB crystallin. All proteins and antibodies were purchased from suppliers as listed in Table 3.

TABLE 3
List of proteins and antibodies used in the protein pin arrays assays

| | | | Protein |
|---|---|---|---|
| Proteins | Catalogue No. | Supplier | used/well |
| Bovine β_{H} crystallin | SPP-235 | Stressgen Inc., BC, | 0.05 μmoles |
| | | Canada | |
| Human γD crystallin | Recombinant, | — | 0.05 μmoles |
| | purified | | |
| Equine Alcohol | 05646 | Sigma-Aldrich, MO | 0.05 μmoles |
| dehydrogenase | | | |
| Porcine Citrate | 103 381 | Roche Diagnostics, | 0.05 μmoles |
| synthase | | IN | |

| | Catalogue | | Dilution |
|---|---|---|---|
| Antibodies | No. | Supplier | used |
| Mouse anti-β crystallin | SPA-230 | Stressgen Inc., BC, | 1:1,000 |
| | | Canada | |
| Mouse anti-γ crystallin | Custom | — | 1:1,000 |
| Rabbit anti-Alcohol | AB1202 | Chemicon | 1:40,000 |
| dehydrogenase | | International, CA | |
| Mouse anti-Citrate synthase | RDI-CBL | RDI Diagnostics | 1:1,000 |
| | 249 | Inc., NJ | |

Column 1 lists the name of the purchased or synthesized protein or antibody, Column 2 lists the catalogue number of the purchased or synthesized protein or antibody, Column 3 lists the supplier of the purchased protein or antibody and Column 4 lists the concentration of the protein or antibody used in the pin array assay.

Human Myoglobin did not interact with the αB crystallin peptides and was the negative control for the protein pin array assay. Ghosh and Clark, Protein Sci 14: 684-95, 2005. Positive interactions resulted in blue color in the wells of the ELISA plate. The intensity of the blue color in the wells was measured at 450 nm using an ELISA plate-reader (BioTek, Winooski, Vt.). The intensity of the blue color (plotted on the X-axis) was a measure of the interaction between αB crystallin peptides (plotted on the Y-axis) and target proteins. To measure the effect of temperature on the interactions between interactive peptides and the target proteins, the target proteins were heated in a water-bath at 45° C. for fifteen minutes prior to use. Pin arrays are unable to differentiate between monomers, dimers or oligomers of target proteins that exist in solution. Instead, pin arrays are very sensitive detectors of interactions between individual peptides and the entire population (monomers, dimers or oligomers) of specific target proteins that may exist in solution under specific conditions. Each target protein was assayed two to five times. A single pin array was used for all experiments and no change in interactions was observed after more than thirty repetitions. The last three peptides of the protein pin array, ₁₆₃REEKPAVT₁₇₀, ₁₆₅EKPAVTAA₁₇₂, ₁₆₇PAVTAAPK₁₇₄, correspond to the epitope (₁₆₃REEKPAVTAAPKK₁₇₅) recognized by the primary antibody for human αB crystallin. A positive reaction is observed for these three peptides in the absence of human αB crystallin as the ligand due to direct binding of the anti-human αB crystallin antibody to these three peptides. The loss of efficiency for the pin array was measured using this assay. The loss of efficiency for the pin array was determined to be <5% by after more than 30 assays.

Molecular modeling of human αB crystallin. The homology modeling program Molecular Operating Environment (MOE) (Chemical Computing Group, Inc, Montreal, Canada) was used to construct the 3-dimensional homology model of human αB crystallin as described previously. Ghosh and Clark, Protein Sci 14: 684-95, 2005. The software included modules for multiple sequence alignment, structure superposition, contact analysis, fold identification, analysis of the stereochemical quality of the predicted models which takes into account parameters like planarity, chirality, phi/psi preferences, chi angles, non-bonded contact distances, unsatisfied donors and acceptors. Fechteler et al., J Mol Biol 253: 114-31, 1995. The wheat sHSP 16.9 crystal structure was chosen as the template for the homology modeling of human αB crystallin because the wheat sHSP16.9 has the highest degree of sequence similarity with human αB crystallin (40% in the α crystallin core domain and 25.4% overall) of all the available crystal structures of sHSPs. In building the homology model of human αB crystallin, the primary sequence of human αB crystallin was aligned with the template protein, wheat sHSP 16.9 primary sequence using ClustalX. Jeanmougin et al., Trends Biochem Sci 23: 403-5, 1998; Aiyar, Methods Mol Biol 132: 221-41, 2000. The predicted secondary structure of human αB crystallin was then obtained (JPred) and verified with the available spin labeling information for the structural elements. Koteiche and McHaourab, J Mol Biol 294: 561-77, 1999. The secondary structure of human αB crystallin was then aligned structurally with the observed secondary structure of the wheat sHSP 16.9. This alignment was used to create a series of ten energy minimized models in MOE. Each model was evaluated using the ModelEval module of MOE and the best fit was selected as the final model and verified by Procheck. Morris et al., Proteins 12: 345-64, 1992. The αB crystallin 3D model computed on the basis of the X-ray crystal structures of wheat sHSP 16.9 and Mj sHSP 16.5 was consistent with the electron spin resonance (ESR) data and previous homology models of αB crystallin. Koteiche and McHaourab, J Mol Biol 294: 561-77, 1999; Guruprasad and Kumari, Int J Biol Macromol 33: 107-12, 2003; Berengian et al., Biochemistry 36: 9951-7, 1997; Koteiche et al., Biochemistry 37: 12681-8, 1998; Muchowski et al., J Mol Biol 289: 397-411, 1999. When the 3D homology model of αB crystallin was superimposed on the crystal structures of wheat sHSP 16.9 and Mj sHSP16.5, the Cα root mean square deviation of the fit was 3.25 Å. Ghosh and Clark, Protein Sci 14: 684-95, 2005. Superimposition of the conserved α crystallin core domains of the three structures resulted in a Cα root mean square deviation of 2.06 Å. Hydrophobic surface areas formed by the chaperone sequences were calculated by a custom script provided by the manufacturer (Chemical Computing Group, Inc, Montreal, Canada). Graphical representations on human alphaB crystallin were made using PyMol and MOE.

### Example 7

Interactive Sites for β_{H} and γD Crystallin in αB Crystallin

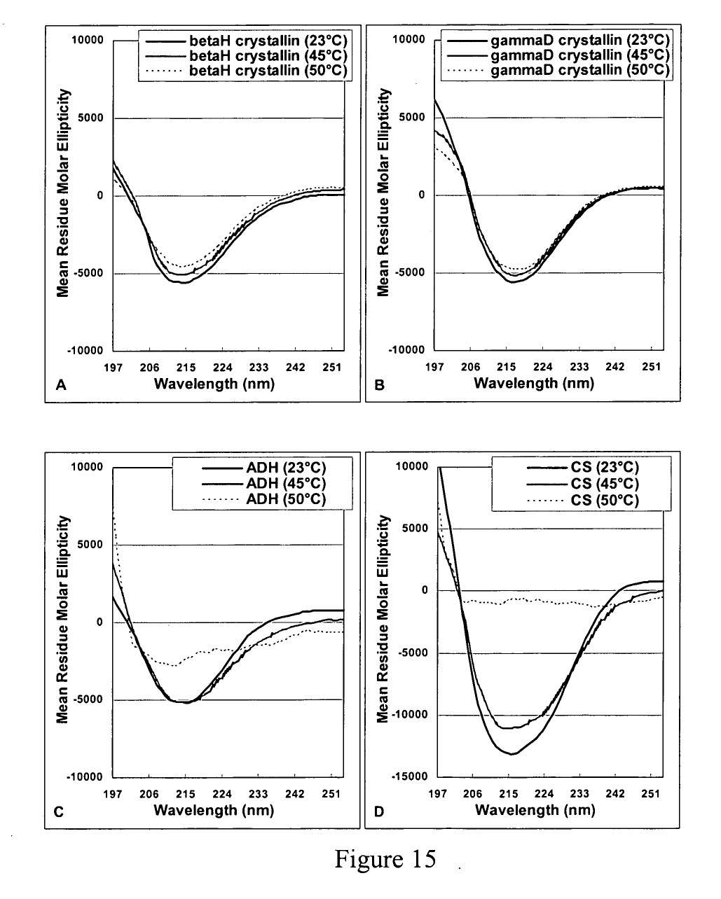

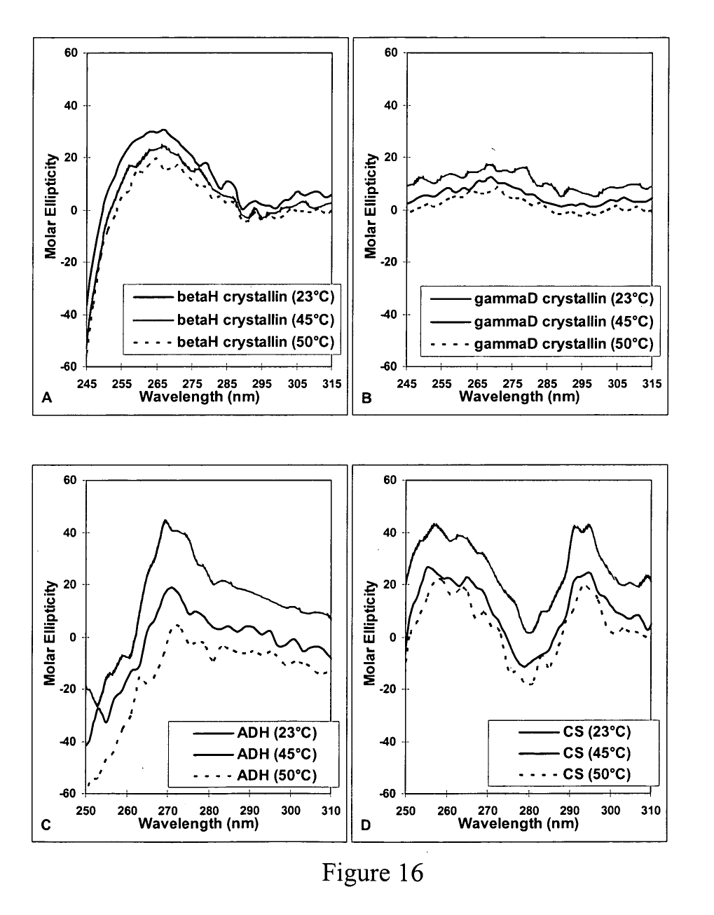

Protein pin arrays enabled the identification of interactive sequences necessary for the chaperone activity of human αB crystallin. Interactions between immobilized 8-mer human αB crystallin peptides and unheated and pre-heated β_{H} crystallin, a natural protein constitute of lens cells, were measured as absorbances at λ=450 nm at 23° C. Maximum absorbances were measured for the peptide sequences, ₉WIRRPFFP₁₆, ₄₅SPFYLRPP₅₂, ₄₇FYLRPPSF₅₄, ₅₁PPSFLRAP_{58 69}RLEKDRFS₇₆, ₇₅FSVNLDVK₈₂, ₈₉LKVKVLGD₉₆, ₁₁₃FISREFHR₁₂₀, ₁₂₁KYRIPADV_{128,} ₁₃₁LTITSSLS₁₃₈, ₁₄₁GVLTVNGP₁₄₈ and ₁₅₇RTIPITRE₁₆₄ when unheated β_{H} crystallin was the target protein (FIG. 13: striped bars). Similar absorbance maxima were measured for interactions between αB crystallin peptides and β_{H} crystallin that was pre-heated at 45° C. for fifteen minutes (FIG. 13: clear bars). αB crystallin peptides that had positive interactions with unheated and pre-heated β_{H} crystallin were identical and differed only in the magnitude of the observed absorbances (FIG. 13: solid black bars). For all sequences in the pin array, eighty of eighty-four αB crystallin peptides had higher absorbances with pre-heated β_{H} crystallin than with unheated β_{H} crystallin (A₄₅₀ₙₘ@45° C.-A₄₅₀ₙₘ@23° C.>0), while the absorbances of the remaining four of eighty-four peptides were similar for pre-heated and unheated β_{H} crystallin (A₄₅₀ₙₘ@45° C.-A₄₅₀ₙₘ@23° C.˜0). Peptides with maximum absorbance were flanked on either side by one or two overlapping peptides with lower absorbances giving the appearance of a peak. The overlapping flanking peptides were shifted toward the N- or C-termini by two amino acids from the peptide recording the maximum absorbance. Peptides with the maximum difference in magnitude of absorbance (A₄₅₀ₙₘ@45° C.-A₄₅₀ₙₘ@23° C.) in each peak were listed in Table 4: Column 2. Far UVCD spectroscopy indicated that the loss of secondary structure of β_{H} crystallin upon heating at 45° C. for fifteen minutes was <10%, and the ellipticity value of the major absorption peaks in the near UVCD spectrum of pre-heated β_{H} crystallin decreased by less than 20% (FIGS. 15a and 16a).

FIG. 13 shows a pattern of interactions between human αB crystallin 8-mer peptides immobilized on pins and unheated β_{H} crystallin at 23° C. and β_{H} crystallin pre-heated at 45° C. for fifteen minutes. The amino acid sequences of each 8-mer human αB crystallin peptide immobilized sequentially on 84 pins in a 96-well ELISA plate format are listed on the Y-axis. The absorbances measured at 450 nm for the interactions between the αB crystallin peptides and unheated β_{H} crystallin (striped bars) or pre-heated β_{H} crystallin (clear bars) using an ELISA based colorimetric method are listed on the primary X-axis. The length of the bars is proportional to the strength of the interaction of that peptide with unheated or pre-heated human β_{H} crystallin, the longer the bar, stronger the interaction. Interactions were not observed at every peptide and there were distinct patterns of interactions with both unheated and pre-heated β_{H} crystallin. An absorbance value of <0.134 with pre-heated β_{H} crystallin was considered the baseline for non-specific interactions. The interaction of the majority of peptides (56/84) was greater with pre-heated β_{H} crystallin than with unheated β_{H} crystallin. The difference in the measured absorbance (A₄₅₀ₙₘ@45° C.-A₄₅₀ₙₘ@RT) for each peptide represents the increased or decreased interaction of that peptide with pre-heated human β_{H} crystallin relative to unheated β_{H} crystallin (plotted to the right as solid black bars). Overall, the interaction between αB crystallin peptides was greater with pre-heated β_{H} crystallin than with unheated β_{H} crystallin.

When β_{H} crystallin was replaced with γD crystallin, a native protein constituent of lens cytoplasm from the same gene family as the β crystallins, as the ligand in the pin array, twelve interactive sequences were identified (FIG. 14: striped bars). The twelve peptides that recorded maximum absorbances at λ=450 nm with unheated γD crystallin were ₃IAIHHPWI₁₀, ₉WIRRPFFP₁₆, ₂₁PFFPFHSP₂₈, ₂₅DQFFGEHL₃₂, ₄₃SLSPFYLR₅₀, ₄₇FYLRPPSF₅₄, ₅₂SFLRAPSW₅₉, ₇₅FSVNLDVK₈₂, ₁₁₁HGFISREF₁₁₈, ₁₁₇EFHRKYR₁₂₄, ₁₃₁LTITSSLS₁₃₈ and ₁₄₁GVLTVNGP₁₄₈. Maximum absorbances were measured at similar peptide sequences in αB crystallin when γD crystallin was pre-heated at 45° C. for fifteen minutes (FIG. 14: clear bars). The magnitude of the absorbances was higher at fifty-six of eighty-four αB crystallin peptides (FIG. 14: solid black bars) and lower at sixteen of the eighty-four peptides when γD crystallin was pre-heated at 45° C. for fifteen minutes. The measured absorbances for the remaining twelve of eighty-four peptides were similar in magnitude for both unheated and pre-heated γD crystallin. αB crystallin peptides that had the highest absorbances with unheated or pre-heated γD crystallin were flanked on either side by one or two overlapping peptides with lower absorbances giving the appearance of a peak. The overlapping flanking peptides were shifted toward the N- or C-termini by two amino acids from the peptide recording the maximum absorbance. Peptides with the maximum difference in magnitude of absorbance (A₄₅₀ₙₘ@45° C.-A₄₅₀ₙₘ@23° C.) in each peak were listed in Table 4: Column 3. Far UVCD spectroscopy indicated that the loss of secondary structure of γD crystallin upon heating at 45° C. for fifteen minutes was <10%, and the ellipticity value of the major absorption peaks in the near UVCD spectrum of pre-heated γD crystallin decreased by less than 25% (FIGS. 15b and 16b).

FIG. 14 shows a pattern of interactions between human αB crystallin 8-mer peptides immobilized on pins and unheated γD crystallin at 23° C. and γD crystallin pre-heated at 45° C. for fifteen minutes. The amino acid sequences of each 8-mer human αB crystallin peptide immobilized sequentially on 84 pins in a 96-well ELISA plate format are listed on the Y-axis. The absorbances measured at 450 nm for the interactions between the αB crystallin peptides and unheated γD crystallin (striped bars) or pre-heated γD crystallin (clear bars) using an ELISA based colorimetric method are listed on the primary X-axis. The length of the bars is proportional to the strength of the interaction of that peptide with unheated or pre-heated human γD crystallin. Interactions were not observed at every peptide and there were distinct patterns of interactions with both unheated and pre-heated γD crystallin. An absorbance value of <0.348 with pre-heated γD crystallin was considered the baseline for non-specific interactions. The interaction of the majority of peptides (56/84) was greater with pre-heated γD crystallin than with unheated γD crystallin. The difference in the measured absorbance (A₄₅₀ₙₘ@45° C.-A₄₅₀ₙₘ@RT) for each peptide represents the increased or decreased interaction of that peptide with pre-heated human γD crystallin relative to unheated γD crystallin (plotted on the right as solid black bars). Overall, the interaction between αB crystallin peptides was greater with pre-heated γD crystallin than with unheated γD crystallin.

### Example 8

Chaperone Sites for ADH and CS in αB Crystallin

In addition to interactions with physiological target proteins in the β/γ crystallin family, interactions of the αB crystallin peptides with unheated and pre-heated chaperone target proteins, alcohol dehydrogenase (ADH) and citrate synthase (CS) were measured. Prior to use in the pin array assay, the secondary structures of β_{H} crystallin, γD crystallin, ADH, and CS were determined using far ultra-violet circular dichroism (UVCD) at 23° C., after heating at 45° C. for fifteen minutes and after heating at 50° C. for sixty minutes (FIG. 15). The maximum ellipticity (Θₘₐₓ) for β_{H} crystallin was observed to be at λ=214 nm at all three temperatures, and the magnitude of the maximum ellipticity (Θₘₐₓ) decreased from −5576 deg.cm².decimole⁻¹ at 23° C. to −5043 deg.cm².decimole⁻¹ after heating at 45° C. for fifteen minutes and to −4501 deg.cm².decimole₋₁ after heating at 50° C. for sixty minutes (FIG. 15a). Similarly, the Θₘₐₓ for γD crystallin was observed to be at k=217 nm at all three temperatures, and the magnitude of θₘₐₓ decreased from −5571 deg.cm² decimole⁻¹ at 23° C. to −5150 deg.cm² decimole⁻¹ after heating at 45° C. for fifteen minutes and to −4719 deg.cm² decimole⁻¹ after heating at 50° C. for sixty minutes (FIG. 15b). The Θₘₐₓ for ADH was at λ=215 nm and the magnitude of the Θₘₐₓ decreased from −5190 deg.cm².decimole⁻¹ at 23° C. to −5139 deg.cm².decimole⁻¹ after heating at 45° C. for fifteen minutes indicating <1% loss of secondary structure upon heating at 45° C. (FIG. 15c). The magnitude of the Θₘₐₓ of ADH further decreased to −2523 deg.cm².decimole⁻¹ after heating at 50° C. for sixty minutes indicating >50% loss of secondary structure upon heating at 50° C. (FIG. 15c). The Θₘₐₓ for CS was at k=215 nm and the magnitude of the Θₘₐₓ decreased from −13076 deg.cm².decimole⁻¹ at 23° C. to −11070 deg.cm².decimole⁻¹ after heating at 45° C. for fifteen minutes, indicating ˜15% loss of secondary structure upon heating at 45° C. (FIG. 15d). Upon heating CS at 50° C. for sixty minutes, the Θₘₐₓ of CS decreased to −648 deg.cm².decimole⁻¹ indicating >95% loss of secondary structure at 50° C. (FIG. 4d).

FIG. 15 shows a far UVCD of β_{H} crystallin, γD crystallin, alcohol dehydrogenase (ADH) and citrate synthase (CS). Spectra were collected for β_{H} crystallin (A: top left), γD crystallin (B: top right), ADH (C: bottom left) and CS (D: bottom right), at 23° C., 45° C. and 50° C. The UVCD spectra of β_{H} and γD crystallin remained largely unchanged between 23° C.-50° C. (83, 84). The spectra for ADH at 23° C. and 45° C. were similar, while at 50° C. there was a significant decrease in the ellipticity at 215 nm. The spectra for CS showed decreased ellipticity at 215 nm with increasing temperature from 23° C.-50° C. Far UVCD spectra indicated that β_{H} crystallin and γD crystallin were thermostable and remained folded up to 50° C., ADH remained folded up to 45° C. but unfolded at 50° C. CS was the least thermostable of the four chaperone target proteins and unfolded at 45° C. The amount unfolding in response to increase in temperature was as follows: CS>>>ADH>β_{H} crystallin˜γD crystallin.

Prior to use in the pin array assay, the tertiary structures of β_{H} crystallin, γD crystallin, ADH, and CS were determined using near ultra-violet circular dichroism at 23° C., after heating at 45° C. for fifteen minutes and after heating at 50° C. for sixty minutes (FIG. 16). The maximum absorption peak for β_{H} crystallin was at k=267 nm at 23° C. The magnitude of the absorption peak at k=267 nm decreased from 30.71 deg.cm².decimole⁻¹ at 23° C. to 24.22 deg.cm².decimole⁻¹ upon heating β_{H} crystallin at 45° C. for fifteen minutes indicating partial unfolding at 45° C. and to 15.78 deg.cm².decimole⁻¹ upon heating at 50° C. for sixty minutes indicating substantial unfolding at 50° C. (FIG. 16a). The maximum absorption peak for γD crystallin was at k=269 nm at 23° C. The magnitude of the absorption peak at k=269 nm decreased from 17.26 deg.cm².decimole⁻¹ at 23° C. to 12.74 deg.cm².decimole⁻¹ upon heating γD crystallin at 45° C. for fifteen minutes indicating partial unfolding at 45° C. and to 6.75 deg.cm².decimole⁻¹ upon heating at 50° C. for sixty minutes indicating substantial unfolding at 50° C. (FIG. 16b). The maximum absorption peak for ADH was at λ=270 nm at 23° C. The magnitude of the absorption peak at λ=270 nm decreased from 44.27 deg.cm².decimole⁻¹ at 23° C. to 14.23 deg.cm².decimole⁻¹ upon heating ADH at 45° C. for fifteen minutes indicating partial unfolding at 45° C. and to −2.37 deg.cm².decimole⁻¹ upon heating at 50° C. for sixty minutes indicating substantial unfolding at 50° C. (FIG. 16c). The maximum absorption peak for CS was at λ=257 nm at 23° C. The magnitude of the absorption peak decreased at λ=257 nm from 42.54 deg.cm².decimole⁻¹ at 23° C. to 25.20 deg.cm².decimole⁻¹ upon heating CS at 45° C. for fifteen minutes indicating partial unfolding at 45° C. and to 20.95 deg.cm².decimole⁻¹ upon heating at 50° C. for sixty minutes indicating substantial unfolding at 50° C. (FIG. 16d).

FIG. 16 shows a near UVCD of β_{H} crystallin, γD crystallin, ADH and CS. Spectra were collected for β_{H} crystallin (A: top left), γD crystallin (B: top right), ADH (C: bottom left) and CS (D: bottom right) at 23° C., 45° C. and 50° C. The ellipticity of the absorption peaks in the near UVCD spectra of β_{H} crystallin, γD crystallin, ADH and CS decreased by 20-60% upon heating at 45° C. for fifteen minutes.

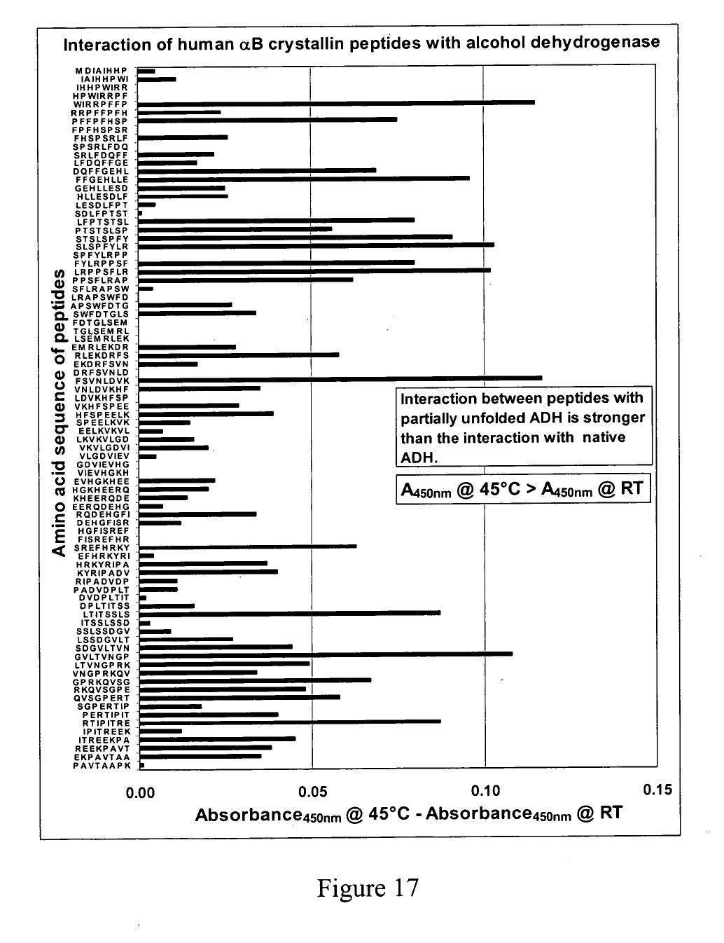

The pattern of absorbance at k=450 nm when αB crystallin peptides were assayed with unheated and pre-heated ADH was similar to the absorbance pattern obtained with unheated and pre-heated β_{H}/γD crystallin (FIG. 17). Maximum absorbances were measured for the αB crystallin peptide sequences ₉WIRRPFFP₁₆, ₁₃ PFFPFHSP₂₀, ₂₇FFGEHLLE₃₄, ₃₇LFPTSTSL₄₄, ₄₃SLSPFYLR₅₀, ₄₈LRPPSFLR₅₅, ₆₉RLEKDRFS₇₆, ₇₅FSVNLDVK₈₂, ₁₁₅SREFHRKY₁₂₂, ₁₃₁LTITSSLS₁₃₈, ₁₄₁GVLTVNGP₁₄₈ and ₁₅₇RTIPITRE₁₆₄ when ADH was used as the ligand in the pin array assay. The absorbance for thirty-four of the eighty-four peptides increased when ADH was pre-heated at 45° C. for fifteen minutes (A₄₅₀ₙₘ@45° C.-A₄₅₀ₙₘ@23° C.>0), while the absorbance for the remaining fifty of the eighty-four peptides was similar for pre-heated and unheated ADH (A₄₅₀ₙₘ@45° C.-A₄₅₀ₙₘ@23° C.˜0). The difference in magnitude of the absorbance of an interactive peptide sequence with pre-heated and unheated ADH (A₄₅₀ₙₘ@45° C.-A₄₅₀ₙₘ@23° C.) was a measure of the increased interaction of that peptide with pre-heated ADH relative to unheated native ADH. αB crystallin peptides that had the highest difference in magnitude of absorbances with pre-heated and unheated ADH (A₄₅₀ₙₘ@45° C.-A₄₅₀ₙₘ@23° C.) were flanked on either side by one or two peptides with lower differences in magnitude of absorbances giving the appearance of peaks. The overlapping flanking peptides were shifted from the peak peptide toward the N- or C-termini by two amino acids from the peptide recording the maximum absorbance. Peptides with the maximum difference in magnitude of absorbance (A₄₅₀ₙₘ@45° C.-A₄₅₀ₙₘ@23° C.) in each peak were listed in Table 4: Column 4. The results confirmed that the interaction between human αB crystallin was greater with pre-heated ADH than with unheated native ADH.

FIG. 17 shows a pattern of interaction between human αB crystallin peptides and ADH. The Y-axis lists the amino acid sequences of the 8-mer peptides that are immobilized sequentially in a 96-well format. The difference in the measured absorbances of each peptide with pre-heated partially denatured ADH and unheated native ADH (A₄₅₀ₙₘ@45° C.-A₄₅₀ₙₘ@RT) represents an increased or decreased interaction of that peptide with partially denatured ADH and native ADH and was represented as a horizontal bar on the X-axis. 34/84 peptides had a stronger interaction with partially denatured ADH than native ADH, while the remaining 50/84 had a similar interaction with either native or partially denatured ADH. The interaction between αB crystallin peptides was greater with pre-heated unfolded ADH than with unheated native ADH. An absorbance difference, A₄₅₀ₙₘ@45° C.-A₄₅₀ₙₘ@RT<0.05 was considered as baseline.

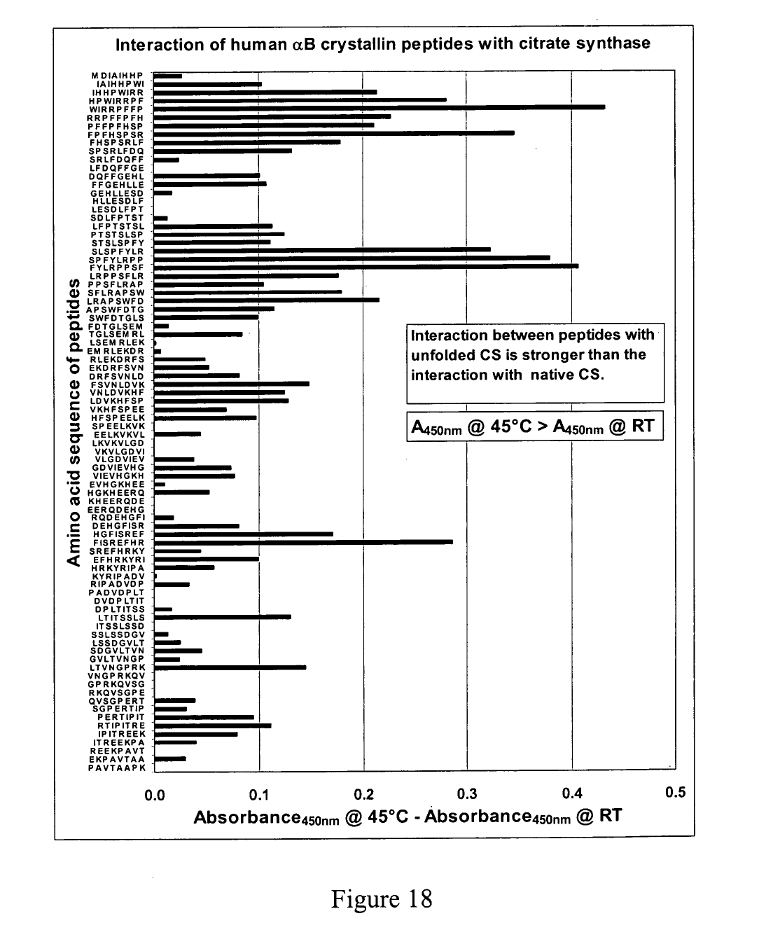

The pattern of absorbance at λ=450 nm when αB crystallin peptides were assayed with unheated and pre-heated CS was similar to the absorbance pattern obtained with unheated and pre-heated β_{H}/γD crystallin and ADH (FIG. 18). Ten sequences in human αB crystallin, ₉WIRRPFFP₁₆, ₂₃FPFHSPSR₃₀, ₄₃SLSPFYLR₅₀, ₄₇FYLRPPSF₅₄, ₅₅LRAPSWFD₆₂, ₇₅FSVNLDVK₈₂, ₁₁₃FISREFHR₁₂₀, ₁₃₁LTITSSLS₁₃₈, ₁₄₃LTVNGPRK₁₅₀ and ₁₅₇RTIPITRE₁₆₄, were identified by their absorbance maxima in the presence of CS as the target protein in the pin array assay. αB crystallin peptides that had the highest differences in magnitude of absorbances with pre-heated and unheated CS (A₄₅₀ₙₘ@45° C.-A₄₅₀ₙₘ@23° C.) were flanked on either side by one or two peptides with lower differences in magnitude of absorbances giving the appearance of peaks. The overlapping flanking peptides were shifted from the peak peptide toward the N- or C-termini by two amino acids from the peptide recording the maximum absorbance. The difference in magnitude of absorbance for each peptide with partially unfolded and native CS (A₄₅₀ₙₘ@45° C.-A₄₅₀ₙₘ@23° C.) represented an increased interaction of that peptide with pre-heated unfolded CS relative to unheated native CS. The interaction of seventy-two of eighty-four αB crystallin peptides increased when CS was pre-heated at 45° C. for fifteen minutes (A₄₅₀ₙₘ@45° C.-A₄₅₀ₙₘ@23° C.>0), while the absorbance for the remaining twelve of eighty-four peptides was similar for both native and partially unfolded CS (A₄₅₀ₙₘ@45° C.-A₄₅₀ₙₘ@123° C.˜0). Peptides with the maximum difference in magnitude of absorbance (A₄₅₀ₙₘ@45° C.-A₄₅₀ₙₘ@23° C.) in each peak were listed in Table 4: Column 5. The increase in the magnitude of the absorbances of the peptides with pre-heated CS indicated that αB crystallin peptides had a stronger interaction with pre-heated partially unfolded CS than with unheated native CS.

FIG. 18 shows a pattern of interaction between human αB crystallin peptides and CS. The Y-axis lists the amino acid sequences of the 8-mer peptides that are immobilized sequentially in a 96-well format. The difference in the measured absorbances of each peptide with pre-heated partially denatured CS and unheated native CS (A₄₅₀ₙₘ@45° C.-A₄₅₀ₙₘ@RT) represents an increased or decreased interaction of that peptide with pre-heated partially denatured CS and unheated native CS and was represented as a horizontal bar on the X-axis. 72/84 peptides had a stronger interaction with partially denatured CS as compared to native CS, while the remaining 12/84 had a similar interaction between native and partially denatured CS. The interaction between αB crystallin peptides was greater with pre-heated unfolded CS than with unheated native CS. An absorbance difference, A₄₅₀ₙₘ@45° C.-A₄₅₀ₙₘ@RT<0.11 was considered as baseline.

TABLE 4
List of αB crystallin peptides that recorded the highest absorbances in the
presence of unheated and pre-heated β_{H} crystallin, γD crystallin, ADH and CS.

| Region | β_{H} crystallin | γD crystallin | ADH | CS | Common |
|---|---|---|---|---|---|
| N-terminus | — | ₃IAIHHPWI₁₀ | — | — | — |
| N-terminus | ₉WIRRPFFP₁₆ | ₉WIRRPFFP₁₆ | ₉WIRRPFFP₁₆ | ₉WIRRPFFP₁₆ | ₉WIRRPFFPFHSP₂₀ |
| N-terminus | — | ₂₁PFFPFHSP₂₈ | ₂₁PFFPFHSP₂₈ | ₂₃FPFHSPSR₃₀ | |
| N-terminus | — | ₂₅DQFFGEHL₃₂ | ₂₇FFGEHLLE₃₄ | — | — |
| N-terminus | — | — | ₃₇LFPTSTSL₄₄ | — | ₄₃SLSPFYLRLPPSFLRAP₅₈ |
| N-terminus | ₄₅SPFYLRPP₅₂ | ₄₃SLSPFYLR₅₀ | ₄₃SLSPFYLR₅₀ | ₄₃SLSPFYLR₅₀ | |
| N-terminus | ₄₇FYLRPPSF₅₄ | ₄₇FYLRPPSF₅₄ | ₄₉LRPPSFLR₅₆ | ₄₇FYLRPPSF₅₄ | |
| N-terminus | ₅₁PPSFLRAP₅₈ | ₅₃SFLRAPSW₆₀ | — | ₅₅LRAPSWFD₆₂ | |
| Core domain | ₆₉RLEKDRFS₇₆ | — | ₆₉RLEKDRFS₇₆ | — | ₇₅FSVNLDVK₈₂ |
| Core domain | ₇₅FSVNLDVK₈₂ | ₇₅FSVNLDVK₈₂ | ₇₅FSVNLDVK₈₂ | ₇₅FSVNLDVK₈₂ | |
| Core domain | ₈₉LKVKVLGD₉₆ | — | — | — | — |
| Core domain | ₁₁₃FISREFHR₁₂₀ | ₁₁₁ HGFISREF₁₁₈ | — | ₁₁₃FISREFHR₁₂₀ | ₁₁₃FISREFHR₁₂₀ |
| Core domain | ₁₂₁KYRIPADV₁₂₈ | ₁₁₇EFHRKYRI₁₂₄ | ₁₁₅SREFHRKY₁₂₂ | — | |
| Core domain | ₁₃₁LTITSSLS₁₃₈ | ₁₃₁LTITSSLS₁₃₈ | ₁₃₁LTITSSLS₁₃₈ | ₁₃₁LTITSSLS₁₃₈ | ₁₃₁LTITSSLS₁₃₈ |
| Core domain | ₁₄₁GVLTVNGP₁₄₈ | ₁₄₁GVLTVNGP₁₄₈ | ₁₄₁GVLTVNGP₁₄₈ | ₁₄₃LTVNGPRK₁₅₀ | ₁₄₁GVLTVNGP₁₄₈ |
| C-terminus | ₁₅₇RTIPITRE₁₆₄ | — | ₁₅₇RTIPITRE₁₆₄ | ₁₅₇RTIPITRE₁₆₄ | ₁₅₇RTIPITRE₁₆₄ |

In table 4, Column 1 lists the region of αB crystallin where each chaperone sequence is located. Column 2 lists the interactive sequences in αB crystallin for human β_{H} crystallin. Column 3 lists the interactive sequences in αB crystallin for human γD crystallin. Column 4 lists the αB crystallin peptides chaperone sequences for ADH. Column 5 lists the αB crystallin peptides chaperone sequences for CS. Column 6 lists the seven common chaperone sequences that were observed to interact with three or more pre-heated chaperone target proteins.

Seven interactive sequences for chaperone function in αB crystallin, ₉WIRRPFFPFHSP₂₀, ₄₃SLSPFYLRPPSFLRAP₅₈, ₇₅FSVNLDVK₈₂, ₁₁₃FISREFHR₁₂₀, ₁₃₁LTITSSLS₁₃₈, ₁₄₁GVLTVNGP₁₄₈ and ₁₅₇RTIPITRE₁₆₄ were identified as sequences that had the strongest interactions with chaperone the target proteins, ADH and CS, and the physiological proteins, β_{H}/γD crystallin, that were pre-heated at 45° C. for fifteen minutes (Table 4: Column 6). Two of the chaperone sequences are in the N-terminus region, four are non-overlapping chaperone sequences in the conserved α crystallin core domain, and a single non-overlapping chaperone sequence is in the C-terminus extension of αB crystallin.

### Example 9

Chaperone Assays of the Interactive Sequences

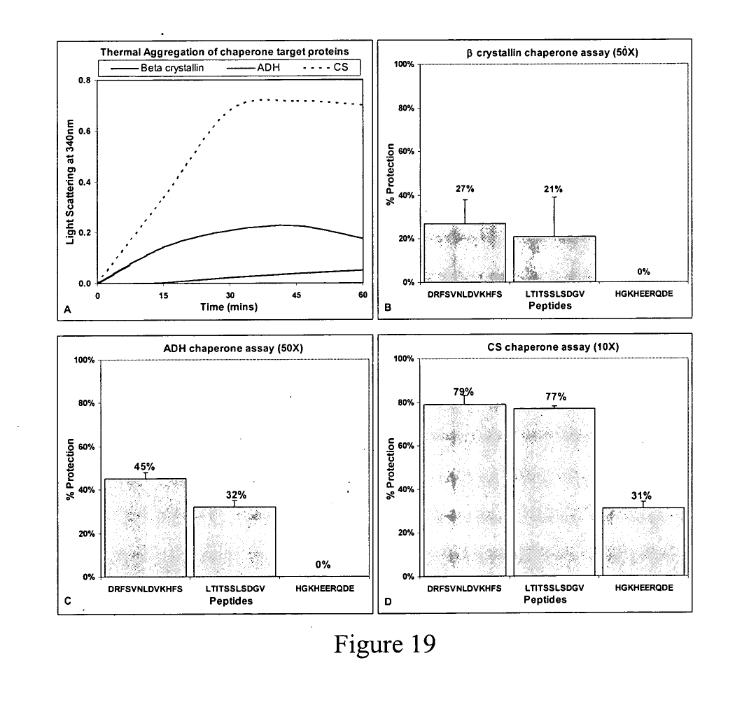

Two αB crystallin sequences, ₇₃DRFSVNLDVKHFS₈₅ and ₁₃₁LTITSSLSDGV₁₄₁, that were in the conserved α crystallin core domain and were observed to have positive interactions with pre-heated target proteins in the pin array were synthesized to determine their chaperone activity in vitro. The chaperone activity of the peptides was measured as their ability to protect against the thermal aggregation of three chaperone target proteins β_{H} crystallin, ADH and CS in chaperone assays performed at 50° C. (FIG. 19). A non-interactive αB crystallin sequence ₁₁₁HGKHEERQDE₁₂₀ was used as the negative control in the chaperone assays (FIG. 19). Based on near and far UVCD, the three target proteins unfolded in the order: CS>>>ADH>>β_{H} crystallin when they were heated at 50° C. for sixty minutes (FIGS. 15 and 16). The thermal aggregation of β_{H} crystallin, ADH and CS was measured as light scattering at λ=340 nm in the presence and absence of each peptide. The chaperone activity of each peptide was calculated as percent protection in which the aggregation of each target protein in the absence of any peptides was set at 0% protection (FIG. 19). The interactive peptides ₇₃DPFSVNLDVKHFS₈₅ and ₁₃₁LTITSSLSDGV₁₄₁ were effective inhibitors of aggregation of all three chaperone target proteins. The greatest protection was observed with CS, the target protein that was the most unfolded upon heating at 50° C. Less protection was observed with the partially unfolded target proteins, β_{H} crystallin and ADH. A 50:1 molar ratio of peptide:β_{H} crystallin/ADH resulted in approximately 30% protection for both β_{H} crystallin and ADH, while a 10:1 molar ratio of peptide:CS resulted in greater than 70% protection of CS against thermal aggregation. No protection against aggregation was observed with the control peptide, ₁₁₁HGKHEERQDE₁₂₀, even at a 50:1 molar ratio.

FIG. 19 shows a chaperone assays of two positive interactive sequences, ₇₃DRFSVNLDVKHFS₈₅ and ₁₃₁LTITSSLSDGV₁₄₁ and a non-interactive sequence, ₁₁₁HGKHEERQDE₁₂₀ from the α crystallin core domain of human αB crystallin (control). A (top left): thermal aggregation of chaperone target proteins β_{H} crystallin, ADH and CS in the absence of peptides was measured as light scattering at λ=340 nm. Each target protein was heated at 50° C. for sixty minutes in the absence or presence of one of the peptides. The amount of aggregation correlated with the amount of unfolding observed with near and far UVCD. β_{H} crystallin, the most thermostable of the three proteins aggregated the least and had an absorbance maximum of 0.05 AU, followed by ADH with an absorbance maximum of 0.23 AU and CS with an absorbance maximum of 0.72 AU in sixty minutes (Unfolding/Aggregation: CS>ADH>β_{H} crystallin). The chaperone activity of each peptide was calculated as percent protection in which the aggregation of each target protein in the absence of any peptides was set at 0% protection. The ability of the ₇₃DRFSVNLDVKHFS₈₅, ₁₃₁LTITSSLSDGV₁₄₁ and ₁₁₁HGKHEERQDE₁₂₀ to protect against the thermal aggregation of β_{H} crystallin (B: top right), ADH (C: bottom left) and CS (D: bottom right) were tested in vitro (vertical bars). A 50:1 molar ratio of peptide:target protein resulted in modest protection of β_{H} crystallin and ADH by the two positive peptides while the control peptide had no protective ability. A 10:1 molar ratio of peptide:target protein was sufficient in conferring significant protection against the aggregation of CS by ₇₃DRFSVNLDVKHFS₈₅ and ₁₃₁LTITSSLSDGV₁₄₁. The control peptide ₁₁₁HGKHEERQDE₁₂₀ conferred partial protection against the aggregation of CS.

### Example 10

Mapping Chaperone Sites to the 3D Structure of Human αB Crystallin

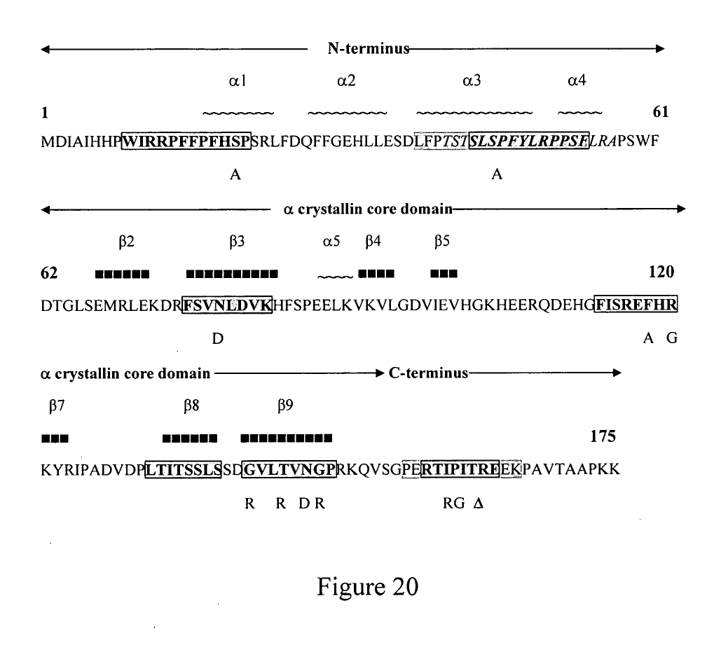

The seven sequences identified as interactive domains for chaperone function in human αB crystallin using protein pin arrays (Table 4: Column 6) were assigned secondary structure based on electron spin resonance (ESR) and homology modeling data (FIG. 20). ₉WIRRPFFPFHSP₂₀ in the N-terminal region, ₁₁₃FISREFHR₁₂₀ in the loop region of the α crystallin core domain and ₁₅₇RTIPITRE₁₆₄ in the C-terminal region were unstructured motifs while ₄₃SLSPFYLR₅₀ (α3) and ₄₇FYLRPPSF₅₄ (α4) formed a helix-turn-helix motif in the N-terminal region. The remaining peptides ₇₅FSVNLDVK₈₂ (β3), ₁₃₁LTITSSLS₁₃₈ (β8) and ₁₄₁GVLTVNGP₁₄₈ (β9) formed three β strand motifs in the α crystallin core domain (FIG. 20).

FIG. 20 shows a comparison of the peptides identified using the human αB crystallin pin arrays with previously reported interactive sequences for αB crystallin. Sequences identified as interactive domains important for chaperone activity using protein pin arrays are in boxes. Subunit-subunit interaction sites identified by the protein pin arrays are shaded in grey. Site-specific mutations that altered the chaperone function of αB crystallin are shown below the residue(s) that were substituted or deleted (Δ). Secondary structure of αB crystallin predicted by ESR and homology modeling is shown in the form of ˜ which represent helices and ▪ which represent β strands. Ghosh and Clark, Protein Sci 14: 684-95, 2005; Guruprasad and Kumari, Int J Biol Macromol 33: 107-12, 2003; Koteiche et al., Biochemistry 37: 12681-8, 1998. All reported point mutations that were observed to have an effect on the chaperone activity of αB crystallin overlapped with the consensus sequences for chaperone function identified by the pin array assays. The sequence identified by Sreelakshmi et al. is in shown in italics. Sreelakshmi et al., Biochemistry 43: 15785-95, 2004.

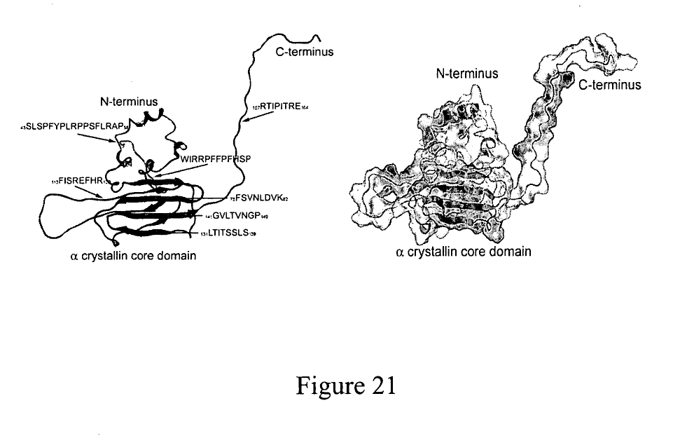

The observed interactive domains were mapped to a computed 3D homology model of human αB crystallin to identify the 3-D structure of the interactive chaperone sequences (FIG. 21a). A space filled model of the human αB crystallin was generated to view and analyze the interactive domains for the chaperone function in αB crystallin (FIG. 21b). The three interactive sequences β3, β8 and β9 appeared to form one of the external surfaces of the α crystallin core domain, a β sandwich structure resembling an immunoglobulin-like fold, which is characteristic of small heat shock proteins. The β strand residues that were oriented internally stabilized the structure of the β sandwich. Side chains that were oriented externally contributed to the interactions with chaperone target proteins. Residues in the N- and C-termini did not appear to be involved directly in the tertiary structure of the α crystallin core domain. The core domain sequence ₁₁₃FISREFHR₁₂₀ formed part of the loop region that connected the two β sheets of the core domain and contributed to its structural integrity. The accessible surface area of the exposed residues of the N-terminal interactive sequences was calculated to be 71% hydrophobic, followed by the C-terminal extension region that was 69% hydrophobic and the α crystallin core domain was 64% hydrophobic in decreasing order. The proportion of hydrophobic surface over the interactive sequences was consistent with the importance of hydrophobic interactions in the recognition of unfolding proteins by sHSP molecular chaperones. Sharma et al., J Biol Chem 275: 3767-71, 2000; Sharma et al., Biochem Biophys Res Commun 239: 217-22, 1997; Sharma et al., J Biol Chem 273: 15474-8, 1998; Sharma et al., J Biol Chem 273: 8965-70, 1998; Singh and Rao Ch, FEBS Lett 527: 234-8, 2002; Srinivas et al., Mol Vis 7: 114-9, 2001; Rajaraman et al., FEBS Lett 497: 118-23, 2001.

FIG. 21 shows a 3-dimensional map of the αB crystallin interactive domains. Interactive domains of human αB crystallin identified by the pin arrays are in red while non-interactive regions are in blue. A (left): ribbon representation of the secondary and tertiary structure of human αB crystallin. The sequences ₉WIRRPFFPFHSP₂₀, ₁₁₃FISREFHR₁₂₀ and ₁₅₇RTIPITRE₁₆₄ did not have secondary structure. The sequence ₄₃SLSPFYLRPPSFLRAP₅₈, formed the α3-turn-α4 motif while the sequences ₇₅FSVNLDVK₈₂, ₁₃₁LTITSSLS₁₃₈, ₁₄₁GVLTVNGP₁₄₈ formed β strand motifs β3, β8 and β9 respectively. B (right): solid models of the 3D structure of human αB crystallin. Surfaces of residues in the interactive domains ₉WIRRPFFPFHSP₂₀, ₄₃SLSPFYLRPPSFLRAP₅₈, ₇₅FSVNLDVK₈₂, ₁₁₃FISREFHR₁₂₀, ₁₃₁LTITSSLS₁₃₈, ₁₄₁GVLTVNGP₁₄₈ and ₁₅₇RTIPITRE₁₆₄ that are solvent accessible are in pink. The surfaces of residues in the non-interactive regions of αB crystallin are in grey. Seventy-two percent of the collective accessible surface area of the N-terminal sequences ₉WIRRPFFPFHSP₂₀ and ₄₃SLSPFYLRPPSFLRAP₅₈ was hydrophobic. 67% of the collective accessible surface area of the α crystallin core domain sequences, ₇₅FSVNLDVK₈₂ (β3), ₁₃₁LTITSSLS₁₃₈ (β8) and ₁₄₁GVLTVNGP₁₄₈ (β9) was hydrophobic. The loop region sequence ₁₁₃FISREFHR₁₂₀ was 61% hydrophobic and the C-terminal extension sequence ₁₅₇RTIPITRE₁₆₄ was 59% hydrophobic.

### Example 11

Interactive Polypeptide Sequences of Human αB Crystallin Having Chaperone Activity

Small heat shock proteins, sHSPs, are a family of stress proteins and molecular chaperones with molecular weights up to 43 kDa that contain an N-terminus domain variable in length and primary sequence, a conserved α crystallin core domain, and a C-terminal extension domain that contains the highly conserved I-X-I/V amino acid motif. In this report protein pin arrays identified seven interactive sequences ₉WIRRPFFPFHSP₂₀, ₄₃SLSPFYLRPPSFLRAP₅₈, ₇₅FSVNLDVK₈₂, ₁₁₃FISREFHR₁₂₀, ₁₃₁LTITSSLS_{138,} ₁₄₁GVLTVNGP₁₄₈ and ₁₅₇RTIPITRE₁₆₄, as being important for the chaperone activity of human αB crystallin using endogenous target proteins β_{H}/γD crystallins and non-physiological targets ADH and CS. Although, it is possible that interactions of αB crystallin with native β_{H} crystallin and γD crystallin that require secondary structure might evade detection by the protein pin arrays, it is likely that the pin arrays identified most sequences involved in the interactions of αB crystallin with native β_{H} crystallin and γD crystallin. The interactive peptides identified by the pin arrays were not the most hydrophobic peptides in the human αB crystallin protein pin array. Based on the hydrophobicity values provided by the manufacturer, the interactive peptides, ₉WIRRPFFPFHSP₂₀, ₄₃SLSPFYLRPPSFLRAP₅₈ and ₁₃₁LTITSSLS₁₃₈ were quite hydrophobic. Fourteen non-interactive peptides in the human αB crystallin pin array were more hydrophobic than the remaining interactive peptides, ₇₅FSVNLDVK₈₂, ₁₁₃FISREFHR₁₂₀, ₁₄₁GVLTVNGP₁₄₈ and ₁₅₇RTIPITRE₁₆₄. Ghosh and Clark, Protein Sci 14: 684-95, 2005.

Far UVCD analysis indicated that there was <10% loss of secondary structure of β_{H} crystallin and γD crystallin when they were heated at 45° C. for fifteen minutes. However, the magnitude of the absorption peaks in the near UVCD spectra of β_{H} crystallin and γD crystallin decreased by ˜20-25%, which indicated conformational changes in the tertiary structures of those proteins. In the absence of significant unfolding and loss of secondary structure of β_{H} crystallin and γD crystallin as determined by far UVCD, the increased interaction between interactive αB crystallin peptides and pre-heated β_{H} crystallin and γD crystallin suggested that the interactive domains of αB crystallin detected conformational changes in tertiary structure that resulted from pre-heating β_{H} crystallin and γD crystallin. It appeared that the pin arrays are as sensitive as near UVCD in detecting perturbations in the tertiary structure of unfolded proteins.

Two of the seven chaperone sequences were in the N-terminus, four in the conserved α crystallin core domain, and one in the C-terminal extension containing the highly conserved I-X-IIV amino acid motif. The pin array results suggested that point mutations within the interactive domains can be expected to modify the chaperone activity of αB crystallin and point mutations outside the interactive domains can be expected to have little or no effect on chaperone activity. While systematic studies have not been reported for the sequence motifs identified using the pin arrays to date, literature results are consistent with the suggestion that the sequences identified by the pin arrays are important motifs for the chaperone-like activity of human αB crystallin. Muchowski et al., J Mol Biol 289: 397-411, 1999; Gupta and Srivastava, Invest Ophthalmol Vis Sci 45: 206-14, 2004; Liao et al., Biochem Biophys Res Commun 297: 309-16, 2002; Kumar et al., J Biol Chem 274:24137-41, 1999; Horwitz et al., Int J Biol Macromol 22: 263-9, 1998; Plater et al., J Biol Chem 271: 28558-66, 1996; Derham et al., Eur J Biochem 268: 713-21, 2001. Mutagenesis of αB crystallin in which deletions of the C-terminus extension included Arginine 157 resulted in diminished chaperone-activity in vitro when compared to full-length αB crystallin. Thampi and Abraham, Biochemistry 42: 11857-63, 2003. Arginine 157 is present in the ₁₅₇ RTIPITRE₁₆₄ chaperone sequence identified by the pin arrays. X-ray solution scattering on a truncation mutant of αB crystallin (αB crystallin 57-157) indicated that the α crystallin domain in the absence of the N- and C-terminal extensions formed dimers and had decreased chaperone-like activity in vitro as compared to full-length αB crystallin. Feil et al., J Biol Chem 276: 12024-9, 2001. When two consensus chaperone sequences ₇₃DRFSVNLDVKHFS₈₅ and ₁₃₁LTITSSLSDGV₁₄₁ belonging to the α crystallin core domain were synthesized and tested for protection against thermal unfolding and aggregation of chaperone target proteins β_{H} crystallin, ADH and CS in vitro, a substantial protective effect was observed. The chaperone assays confirmed that the sequences identified using the pin array were important for the chaperone activity of αB crystallin and were consistent with an earlier study in which hydrophobic probes and chaperone assays identified the αB crystallin sequence ₇₃DRFSVNLDVK₈₂ as an interactive sequence for chaperone activity. Sharma et al., J Biol Chem 279: 6027-34, 2004. Selected point or combination mutations in the interactive sequences of αB crystallin can be expected to improve or diminish chaperone activity. A higher concentration of both peptides was required to protect against the aggregation of β_{H} crystallin and ADH, than to protect against the aggregation of CS. Circular dichroism analysis indicated that β_{H} crystallin was partially unfolded and both ADH and CS were almost completely unfolded at 50° C. Taken together, the chaperone assay and circular dichroism data suggested that the peptides were more efficient in protecting against the aggregation of a completely unfolded protein and less efficient in protecting against the aggregation of partially unfolded or native-like proteins.

The interactive sequences identified using the pin arrays were mapped onto a 3-dimensional model of αB crystallin to analyze the structural topography of the chaperone interface. Ghosh and Clark, Protein Sci 14: 684-95, 2005. In the absence of an X-ray crystal or NMR structure, it was observed that superposition of the computed αB crystallin homology model with the crystal structure of Mj sHSP16.5 and wheat sHSP16.9 was remarkable with a Cα root mean square deviation of 2.06 Å for the α crystallin core domain. Kim et al., Nature 394: 595-9, 1998; van Montfort et al., Nat Struct Biol 8: 1025-30, 2001. Secondary structure was assigned to the interactive sequences identified by the pin arrays on the basis of electron paramagnetic resonance data (EPR) and a multiple sequence alignment of human αB crystallin with the crystal structures of wheat sHSP16.9 and Mj sHSP16.5. Kim et al., Nature 394: 595-9, 1998; van Montfort et al., Nat Struct Biol 8: 1025-30, 2001; Koteiche and McHaourab, J Mol Biol 294: 561-77, 1999; Koteiche et al., Biochemistry 37: 12681-8, 1998. The N-terminal chaperone sequence ₉WIRRPFFPFHSP₂₀ was unstructured and formed a surface that was 70% hydrophobic while the sequence ₄₃SLSPFYLRPPSF₅₄ formed a helix-turn-helix motif with an external surface that was 72% hydrophobic and favorable for binding exposed hydrophobic patches of unfolding proteins. Three of the four sequences in the α crystallin core domain, ₇₅FSVNLDVK₈₂ (β3), ₁₃₁LTITSSLS₁₃₈ (β8) and ₁₄₁GVLTVNGP₁₄₈ (β9) were (strands and formed a surface that was 67% hydrophobic. The C-terminal chaperone sequence ₁₅₇RTIPITRE₁₆₄ containing the highly conserved I-X-I/V motif was unstructured and formed a surface that was 59% hydrophobic. The chaperone sequences ₄₃SLSPFYLRPPSF₅₄, ₇₅FSVNLDVK₈₂, ₁₃₁LTITSSLS₁₃₈, ₁₄₁GVLTVNGP₁₄₈ and ₁₅₇RTIPITRE₁₆₄ identified in this report overlapped significantly with sequences identified previously as subunit-subunit interaction sites in αB crystallin using protein pin arrays (FIG. 20: shaded residues). Ghosh and Clark, Protein Sci 14: 684-95, 2005. The sequence ₄₃SLSPFYLRPPSF₅₄ identified by the pin arrays is a subset of the sequence ₄₂TSLSPFYLRPPSFLRA₅₇, previously reported as an interactive region in αB crystallin that interacts with human αA crystallin. Sreelakshmi et al., Biochemistry 43: 15785-95, 2004; Lentze and Narberhaus, Biochem Biophys Res Commun 325: 401-7, 2004. Both synthesized peptides, ₇₃DRFSVNLDVKHFS₈₅ and ₁₃₁LTITSSLSDGV₁₄₁ that protected β_{H} crystallin, ADH and CS from aggregation were previously identified as interactive sequences for subunit-subunit interactions in αB crystallin. Ghosh and Clark, Protein Sci 14: 684-95, 2005. The results suggested that interactive sequences in αB crystallin may have dual roles in subunit assembly and chaperone function, which is consistent with previous mutagenesis data in which loss of chaperone function was accompanied by decreased assembly size. Saha and Das, Proteins 57: 610-7, 2004; Feil and Augustin, Biochem Biophys Res Commun 247: 38-45, 1998. The identification of common sequences for subunit-subunit interactions and chaperone activity in αB crystallin by the pin arrays suggests that dissociation of αB crystallin complexes may be required for the exposure of chaperone sequences that can bind unfolding chaperone target proteins. This hypothesis is consistent with previous studies of the role of oligomeric equilibrium in regulating the chaperone activity of HSP27 and αB crystallin. Shashidharamurthy et al., J Biol Chem 280: 5281-9, 2005; Srinivas et al., Mol Vis 11: 249-55, 2005. Shashidharamurthy et al. reported that dissociation of the HSP27 oligomer was required for interactions with destabilized T4 lysozyme mutants. Shashidharamurthy et al., J Biol Chem 280: 5281-9, 2005. Srinivas et al. reported that arginine hydrochloride enhanced the chaperone activity of αB crystallin by increasing its subunit-subunit dynamics. Srinivas et al., Mol Vis 11: 249-55, 2005.

The lack of interactive sequences similar to the αB crystallin N-terminal and C-terminal chaperone sequences, ₉WIRRPFFPFHSP₂₀, ₄₃SLSPFYLRPPSFLRAP₅₈ and ₁₅₇RTIPITRE₁₆₄, in sHSPs of C. elegans (sHSP12.2, sHSP12.3 and sHSP12.6) could account for the absence of chaperone-like activity of C. elegans sHSPs in vitro. Kokke et al., FEBS Lett 433: 228-32, 1998. The interface formed by the α crystallin core domain peptides ₇₅FSVNLDVK₈₂ (β3), ₁₃₁LTITSSLS₁₃₈ (β8), ₁₄₁GVLTVNGP₁₄₈ (β9) that interacted with all four chaperone target proteins and collectively formed an external surface that was 67% hydrophobic was previously identified as the interface for the assembly of human αB crystallin subunits using pin arrays. Ghosh and Clark, Protein Sci 14: 684-95, 2005. The interface provides residues for both hydrophobic and hydrophilic interactions with native proteins (αA and αB crystallin) as well as with unfolding chaperone target proteins (β_{H} crystallin, γD crystallin). The structure of the α crystallin core domain is highly conserved in the small heat shock protein family and sequences homologous to the αB crystallin chaperone sequences ₇₅FSVNLDVK₈₂, ₁₁₃FISREFHR₁₂₀, ₁₃₁LTITSSLS₁₃₈, ₁₄₁GVLTVNGP₁₄₈ in other small heat shock proteins are expected to be involved in the chaperone function of other sHSPs. In summary, pin array assays, in vitro chaperone assays and circular dichroism spectroscopy of target proteins identified the sequences in full-length αB crystallin that were responsible for interactions with a broad range of target proteins including proteins that are almost completely unfolded (ADH and CS) and proteins that are partially unfolded (γ_{H} crystallin and γD crystallin). Protein pin arrays were effective in identifying protein—protein interactive domains in human αB crystallin that were important in oligomeric assembly and in interactions with unfolding chaperone target proteins. Further investigation of the specific sequences and 3-dimensional structures of the interactive domains in physiologically relevant small heat shock proteins including human sHSP27 and Mycobacterium tuberculosis sHSP 16.3, will provide new information on the function of sHSPs and molecular chaperones in disease. The current results suggest that the collective response of sHSPs to protein unfolding involves several interactive domains and that sHSPs are exquisitely sensitive to protein unfolding. These results could account for the selectivity and sensitivity of small heat shock proteins and their adaptation to the needs of specific cells and their response to stress.

### Example 12

Effect of αB Crystallin and Five αB Crystallin Derived Peptides on the Fibrillization of Aβ or γD Crystallin

Aβ Thioflaviin T fluorescence. Aβ fibrils were grown in the presence and absence of αB crystallin and peptides in conditions similar to that previously described for the presence of αB crystallin. Bakthisaran et al., Biochem J, 2005. Peptides were dissolved to 9.1 mM in trifluorethanol (TFE) and diluted to 0.91 mM stock solutions in 50 mM PBS, 100 mM NaCl, pH 7.4. 3.5 mg of Thioflavin T was dissolved in 100 μl of 50 mM Glycine pH 8.5. A stock solution of 1 mg/ml (0.22 mM) Aβ was prepared. 20 μl Aβ, 50 μl chaperone, 2 μl Thioflavin T was mixed with 28 μl 50 mM PBS, 100 mM NaCl, pH 7.4. Fluorescence was read using a Beckman Coultier DTX 880 multimode detector. Samples were heated at 50° C. for 72 hours and fluorescence was read again.

γD crystallin Thioflaviin T fluorescence. γD crystallin fibrils were formed in a similar way to that of γ crystalline. Meehan et al., J Biol Chem 279: 3413-3419, 2004. Peptides were dissolved to 9.1 mM in TFE and diluted to 0.91 mM stock solutions in 10% TFE, pH 2.0. 3.5 mg of Thioflavin T was dissolved in 100 μl of 50 mM Glycine pH 8.5. A stock solution of 1 mg/ml (0.22 mM) Aβ was prepared. 20 μl Aβ, 5 μl chaperone, 2 μl Thioflavin T was mixed with 73 μl 10% TFE, pH 2.0. Fluorescence was read using a Beckman Coultier DTX 880 multimode detector. Samples were heated at 50° C. for 72 hours and fluorescence was read again.

Chaperone Assays. Chaperone assays of the five peptides were performed using previously established methods with minor modifications. Muchowski et al., J Mol Biol 289: 397-411, 1999. A 1:1 monomeric molar ratio of chaperone:target protein was determined to be the optimum ratio and all subsequent chaperone assays of the β3 mutants were performed in triplicate and at a 1:1 monomeric molar ratio of chaperone:target protein in a 96-well ELISA micro-titer plate. 0.1 mmoles of the chaperone and β_{L} crystallin were mixed in a total volume of 200 μl buffer (5 mM PBS, pH 7.0). Light scattering at λ=340 nm was measured using a Multiskan MCC/340 plate reader before heating (time=0). After the first reading, the plate was heated at 50° C. and readings were taken at fifteen minute intervals for one hour.

Table 5 shows the fluorescence of Aβ or γD crystallin in the absence or presence of wild type αB crystallin and five αB crystallin derived peptides.

TABLE 5

| | Mol. | | Δ_{fluorescence} |
|---|---|---|---|
| | Wt. | Δ_{fluorescence} (Aβ) | (γD crystallin) |
| No chaperone | — | 1,324,974,048 | 888,803,840 |
| αB crystallin | 20159 | 318,471,040 | 1,079,141,056 |
| STSLSPFYLRPPSFLRAP | 2036 | 478,389,568 | 606,290,176 |
| DRFSVNLDVKHFS | 1564 | 264,396,672 | 661,038,176 |
| HGKHEERQDE | 1264 | 796,478,624 | 734,394,240 |
| LTITSSLSSDGV | 1092 | 352,301,312 | 675,352,992 |
| ERTIPITRE | 1114 | 576,742,720 | 611,017,728 |

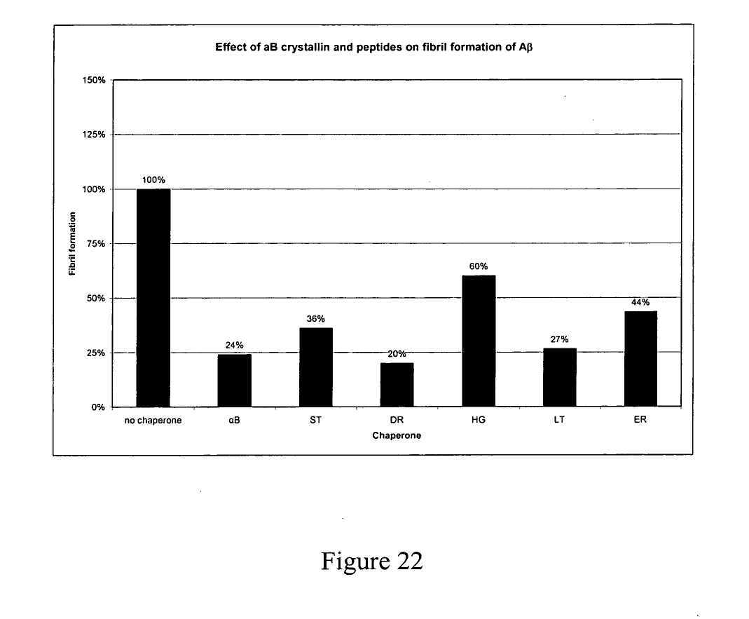

FIG. 22 shows the effect of αB crystallin and five αB crystallin derived peptides on the fibrillization of Aβ. The fibrillization of Aβ was measured as fluorescence of the fluorescent dye Thioflavin T from solutions containing 1:10 monomeric molar ratio of Aβ:peptide. In the absence of Aβ fibrils, Thioflavin T had little or no fluorescence. The fluorescence of the Thioflavin T bound to Aβ fibrils in the absence of any other molecule after 72 hrs was set to 100%. The DR peptide had the strongest effect and the HG peptide had the weakest effect on the fibrillization of Aβ. Fibril formation=Δ_{fluorescence}Aβ (with chaperone)*100/Δ_{fluorescence}Aβ (without chaperone).

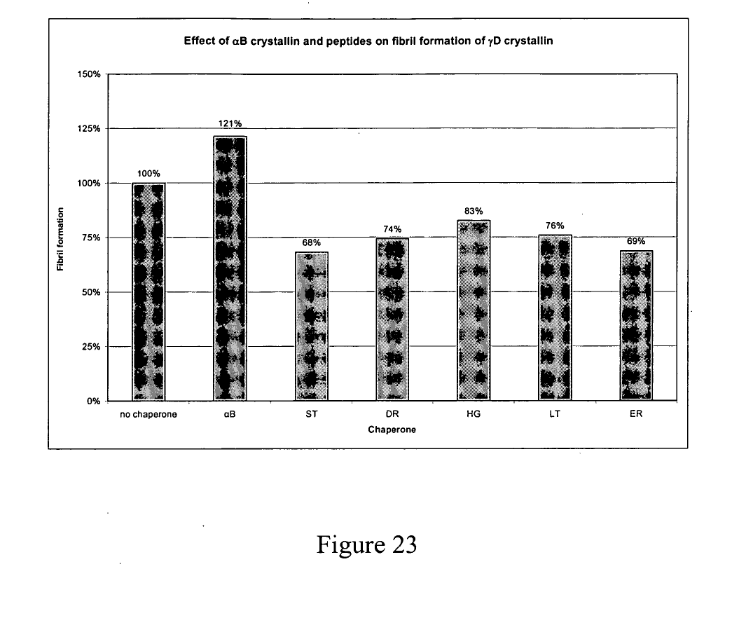

FIG. 23 shows the effect of αB crystallin and five αB crystallin derived peptides on the fibrillization of γD crystallin. The fibrillization of γD crystallin was measured as fluorescence of the fluorescent dye Thioflavin T from solutions containing 1:1 monomeric molar ratio of γD crystallin:peptide. In the absence of fibrils Thioflavin T had little or no fluorescence. The fluorescence of the Thioflavin T bound to γD crystallin fibrils in the absence of any other molecule after 72 hrs was set to 100%. The ST peptide had the strongest effect and wt αB crystallin had the weakest effect on the fibrillization of γD crystallin. Fibril formation=Δ_{fluorescence}γD crystallin (with chaperone)*100/Δ_{fluorescence}γD crystallin (without chaperone).

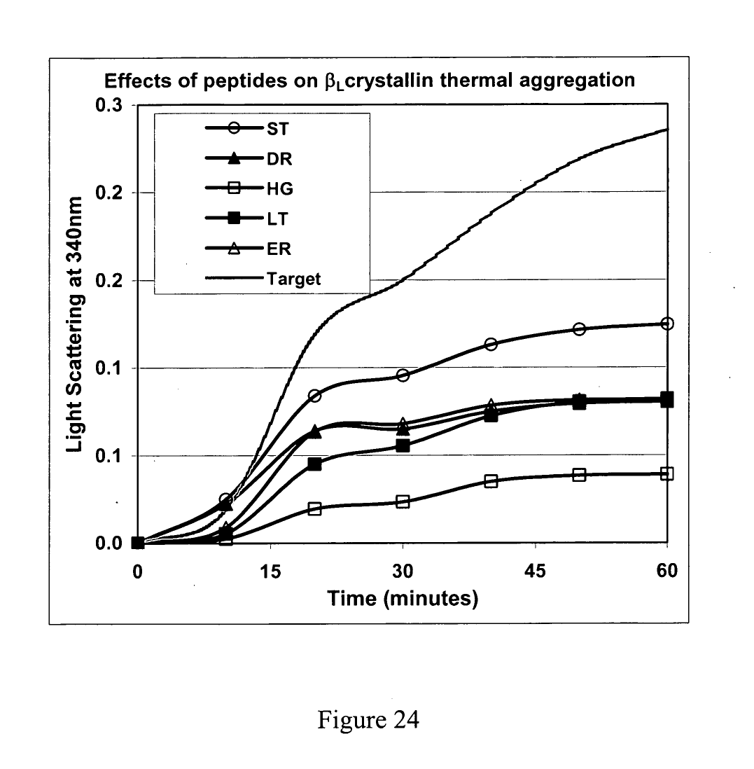

FIG. 24 shows chaperone assays of the five αB crystallin derived peptides. The ability of the peptides to suppress the thermal aggregation of β_{L} crystallin was measured spectrophotometrically as light scattering at λ=340 nm from solutions containing 1:10 monomeric molar ratio of target protein:peptide. In the absence of β_{L} crystallin, the light scattering of the peptides was similar to that of buffer alone indicating that the peptides did not aggregate upon heating. The HG peptide had the strongest effect and the ST peptide had the weakest effect on the aggregation of β_{L} crystallin. The chaperone activities of the DR, LT and ER peptides were identical and slightly lower than the HG peptide.

### Example 13

Chaperone Peptides Inhibit Fibril Formation of Amyloidogenic Proteins

Protein aggregation and fibril formation are the hallmarks of amyloidoses including Alzheimer's disease (AD), Parkinson's disease (PD), senile systemic amyloidosis, hemodialysis-related amyloidosis, and familial amyloidotic neuropathy I. Dobson, C. M., Philos Trans R Soc Lond B Biol Sci 351:133-45, 2001; Sunde et al., J Mol Biol 273:729-39, 1997; Selkoe and Schenk, Annu Rev Pharmacol Toxicol 49:545-84, 2003. The current hypothesis for the cause of these protein conformation diseases is the formation and accumulation of aggregates and fibrils of amyloidogenic proteins including amyloid-β (A,B), α-synuclein, transthyretin (TTR), and β2-microglobulin (β2m). Hardy and Selkoe, Science 297:353-6, 2002; Gorevic et al., J Clin Invest 76:2425-9, 1985; Looi, L. M., Malays J Pathol 17:1-10, 1995. Peptides and peptidomimetics corresponding to the chaperone sequences of molecular chaperones are an attractive strategy for modifying the disease causing mechanisms of local and systemic amyloidoses including Alzheimer's disease, senile systemic amyloidosis, hemodialysis-related amyloidosis, and familial amyloidotic neuropathy I. Amyloid fibrils are highly organized protein/peptide aggregates that are formed by continuous intermolecular f-sheet interactions of partially unfolded proteins or peptides. Fibrillation is distinct from amorphous, or disordered aggregation which can also result from partially or completely unfolded proteins and is the cause of diseases including cataract, desmin-related myopathy, and age-related macular degeneration. Dobson, C. M., Nat Rev Drug Discov 2:154-60, 2003; Dobson, C. M., Semin Cell Dev Biol 15:3-16, 2004. Consistent with their role as endogenous molecules entrusted with protecting cells from the effects of toxic aggregates, significant amounts of low molecular weight chaperones including αB crystallin, sHSP27, sHSP20, and sHSPB2 have been observed in neuritic plaques and Lewy bodies in brain tissues from Alzheimer's disease and Parkinson's disease patients. In addition, low molecular weight molecular chaperones including αB crystallin and sHSP20 inhibit the fibrillation of a number of amyloidogenic proteins including Aβ, β2-microglobulin, α-synuclein, and insulin in vitro. Lee et al., Protein Sci 14:593-601, 2005; Bakthisaran et al., Biochem J, 392:573-581, 2005. The presence of low molecular weight chaperones in both neuritic plaques and Lewy bodies suggests a protective role for molecular chaperones in neurodegenerative diseases. Wilhelmus et al., Acta Neuropathol (Berl), 111: 139-49, 2006; Wilhelmus et al., Neuropathol Appl Neurobiol 32:119-30, 2006; Wilhelmus et al., Brain Res, 1089:67-78, 2006; Dabir et al., Am J Pathol 164:155-66, 2004; Katayama et al., Neuropathol Appl Neurobiol 27:180-8, 2001; Renkawek et al., Acta Neuropathol (Berl) 87:511-9, 1994; Renkawek et al., Acta Neuropathol (Berl) 87:155-60, 1994.

The amyloid cascade pathway, which includes the processes of unfolding, aggregation, and oligomerization (fibril formation) of amyloidogenic proteins, has been the topic of intense research in the field. Hardy and Selkoe, Science 297:353-6, 2002; Hardy J., Curr Alzheimer Res 3:71-3, 2006; Watson et al., Neurol Res 27:869-81, 2005; Ross and Poirier, Nat Med 10 Suppl S10-7, 2004; Huse and Doms, Mol Neurobiol 22:81, 2000; Hardy and Higgins, Science 256:184-5, 1992; Wright et al., Nature 438:878-81, 2005; Bucciantini et al., Nature 416:507-11, 2002; Selkoe, D. J., Physiol Rev 81:741-66, 2001; Ono et al., Neurochem Int. 48:275-85, 2006; Cleary et al., Nat Neurosci 8:79-84, 2005; Hirschfield and Hawkins, Int J Biochem Cell Biol 35:1608-13, 2003; Lesne et al., Nature 440:352-7, 2006; Townsend et al., J Physiol 572:477-492, 2006; Glabe, C. G., Neurobiol Aging 27:570-5, 2006. Current treatment approaches for amyloidosis include preventing the formation of amyloidogenic proteins, stabilizing the non-amyloidogenic form and preventing the fibril formation of amyloidogenic proteins. Molecules that disrupt the amyloid cascade pathway (monomer→oligomer→fibril) including Tramiprosate (Alzhemed™ or Fibrillex™) have so far had the most success in the clinic. Cohen and Kelly, Nature 426:905-9, 2003; Aisen, P. S., CNS Drugs 19:989-96, 2005; Gervais et al., Neurobiol Aging, 2006 May 1 [Epub ahead of print]. Tramiprosate (3-aminopropane-1-sulfonic acid), a glycosaminoglycan-mimetic interferes with the association between glycosaminoglycans and Aβ and is believed to prevent the formation of β sheet fibrils by keeping monomeric Aβ soluble. Gervais et al., Neurobiol Aging, 2006 May 1 [Epub ahead of print]. Tramiprosate is currently in Phase III clinical trials for AD. Another molecule shown to inhibit Aβ fibrillation and Aβ-associated neurotoxicity is the neuropeptide, NAPVSIPQ (NAP), which is a hydrophobic peptide that acts by disrupting the β sheet structure of amyloid fibrils and is currently in Phase II clinical trials for AD. Gozes et al., CNS Drug Rev 11:353-68, 2005; Ashur-Fabian et al., Peptides 24:1413-23, 2003; Zemlyak et al., Regul Pept 96:39-43, 2000. The specific mechanism by which both these molecules target Aβ fibrils limits their potential use as therapeutics in other non-AD amyloidoses. However, small molecules or proteins/peptides that have similar mechanisms of action and are selective rather than specific for only one target protein may serve as the basis for a new class of disease modifying therapeutics for a variety of amyloidoses. Santhoshkumar and Sharma, Mol Cell Biochem 267:147-55, 2004; Gibson and Murphy, Biochemistry 44:8898-907, 2005; DeFelice et al., Faseb J 18:1366-72, 2004; Rzepecki et al., J Biol Chem 279:47497-505, 2004. Although, the use of molecular chaperones as therapeutic molecules in protein conformation diseases is attractive, the large size of these molecules presents significant bioavailability problems in humans. Identification of short peptide sequences in molecular chaperones that interact with the fibril forming regions of amyloidogenic proteins will provide the basis for developing peptide-based therapeutics that inhibit fibril formation and treat various amyloidoses.

Peptide pin array technology and chaperones assays were employed to identify chaperone sequences of the molecular chaperone, human αB crystallin that interact with unfolded proteins and prevent their aggregation. Ghosh et al., Biochemistry 44:14854-69, 2005; Ghosh and Clark, Protein Sci 14:684-95, 2005. In this report, synthesized versions of these five αB crystallin peptides, ₄₁STSLSPFYLRPPSFLRAP₅₈ (ST), ₇₃DRFSVNLDVKHFS₈₅ (DR), ₁₀₁HGKHEERQDE₁₁₀ (HG), ₁₃₁LTITSSLSSDGV₁₄₂ (LT), and ₁₅₆ERTIPITRE₁₆₄ (ER) were evaluated for their ability to inhibit the fibrillation of six amyloidogenic proteins, Aβ, transthyretin, β2-microglobulin, and α-synuclein at stoichometric and sub-stoichometric concentrations. All five βB crystallin peptides inhibited fibrillation of at least one amyloidogenic protein with the HG peptide being the most effective inhibitor of fibril formation. The αB crystallin peptides, STSLSPFYLRPPSFLRAP and HGKHEERQDE were more effective in inhibiting the fibrillation of amyloid-0, the main target for Alzheimer's disease than the two molecules, Tramiprosate and NAP that are currently in human clinical trials for Alzheimer's disease. Ten truncated fragments of the HG peptide achieved almost 100% inhibition of TTR and β2m fibrillation at a 1:1 ratio. Thioflavin T assays demonstrated the effectiveness of inhibiting fibrillation of six amyloidogenic proteins by 4-18 amino acid long peptide sequences of the molecular chaperone αB crystallin.

### Example 14

Effect of Synthesized βB Crystallin Peptides on Aβ₁₋₄₂ Fibril Formation

The effect of five synthesized αB crystallin peptides STSLSPFYLRPPSFLRAP (ST), DRFSVNLDVKHFS (DR), HGKHEERQDE (HG), LTITSSLSSDGV (LT), and ERTIPITRE (ER) on Aβ₁₋₄₂ fibrillation was measured using the ThT fluorescence assay (FIG. 1). The formation and inhibition of Aβ₁₋₄₂ fibrils was qualitatively verified by fluorescence and bright-field microscopy. The concentration and stoichometry of the peptides was varied relative to Aβ₁₋₄₂ to establish a concentration curve. In addition, the effect of two known inhibitors of Aβ₁₋₄₂ fibrillation (APS and NAP) and a control neuropeptide (AT1) with no fibril inhibition activity was measured and compared to the effect of the αB crystallin peptides on Aβ₁₋₄₂ fibrillation. The ThT fluorescence of 30 μM Aβ₁₋₄₂ alone incubated at 37° C. for 48 hrs increased from ˜350,000 to ˜450,000 RFU. 6 μM αB crystallin peptides ST and HG completely inhibited the fibril formation of 30 μM Aβ₁₋₄₂. In contrast, 6 μM DR, LT, and HG had no effect on Aβ₁₋₄₂ fibril formation. 6 μM APS and NAP modestly decreased ThT fluorescence (˜40% decrease), while 6 μM of the negative control peptide AT1 had no effect on Aβ₁₋₄₂ fibril formation. Increasing the concentration of the DR peptide to 30 μM decreased the ThT fluorescence by approximately 50%. At this concentration, no effect on Aβ₁₋₄₂ fibril formation was observed with the LT and ER peptides. However, when the concentrations of the LT and ER peptides were increased to 150 μM, a 60-100% decrease in ThT fluorescence was measured. Increasing the concentration of NAP (30 μM and 150 μM) slightly increased the ThT fluorescence, while increasing the concentration of APS to 150 μM decreased the ThT fluorescence by 100%. The negative control peptide AT1 had no effect on Aβ₁₋₄₂ fibril formation even at a concentration as high as 300 μM. At sub-stoichometric concentrations (6 μM), the αB crystallin peptides STSLSPFYLRPPSFLRAP (ST) and HGKHEERQDE (HG) were more effective than both APS and NAP in inhibiting Aβ₁₋₄₂ fibrillation.

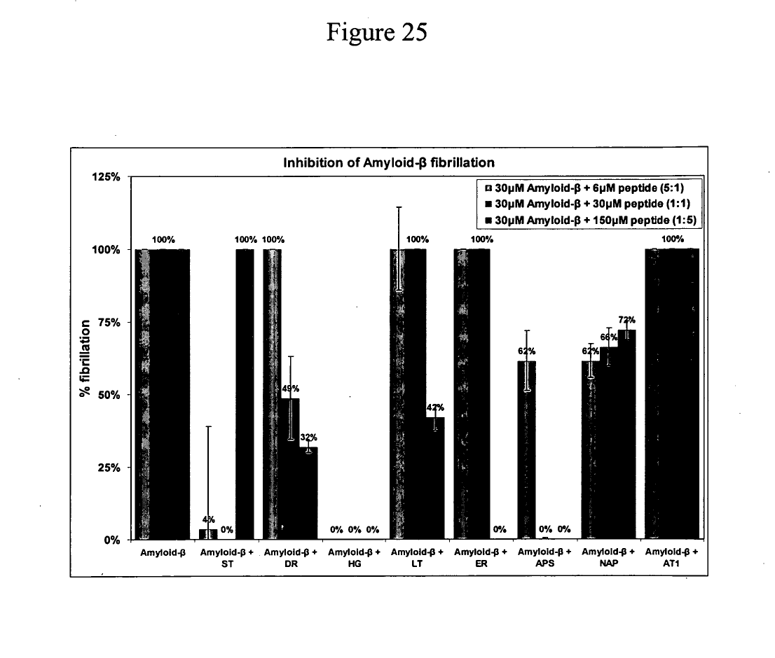

FIG. 25 shows an effect of five αB crystallin peptides on Aβ₁₋₄₂ fibril formation. The Thioflavin T (ThT) fluorescence of Aβ₁₋₄₂ in the presence of varying concentrations of αB crystallin peptides and controls was measured. The addition of 6 μM ST and HG to 30 μM Aβ₁₋₄₂ resulted in a 100% decrease in ThT fluorescence after 48 hrs. The addition of an equivalent concentration (6 μM) of APS and NAP resulted in a 38% decrease in ThT fluorescence. Addition of 6 μM DR, LT, and ER had no effect on the ThT fluorescence of Aβ₁₋₄₂. When the concentration of the peptides was increased to 30 μM (1:1::peptide:Aβ₁₋₄₂), the αB crystallin peptides ST, DR, HG decreased the ThT fluorescence by 50-100%. At the same concentrations, APS and NAP decreased the ThT fluorescence by 30-100%. When the concentration of the peptides was increased further to 150 μM (10:1::peptide:Aβ₁₋₄₂), four of the five αB crystallin peptides DR, HG, LT, and ER decreased the ThT fluorescence by 58-100%. At the same concentrations, APS and NAP decreased the fluorescence by 28-100%. The control peptide AT1 did not show a protective effect at any concentration including a 10:1 peptide:Aβ₁₋₄₂ ratio. The formation and inhibition of Aβ fibrils was verified microscopically. The HG peptide was the most effective inhibitor of Aβ₁₋₄₂ fibril formation and completely prevented the formation of fibrils at 1:5 peptide:Aβ₁₋₄₂ ratio.

### Example 15

Effect of Synthesized αB Crystallin Peptides on Transthyretin Fibril Formation

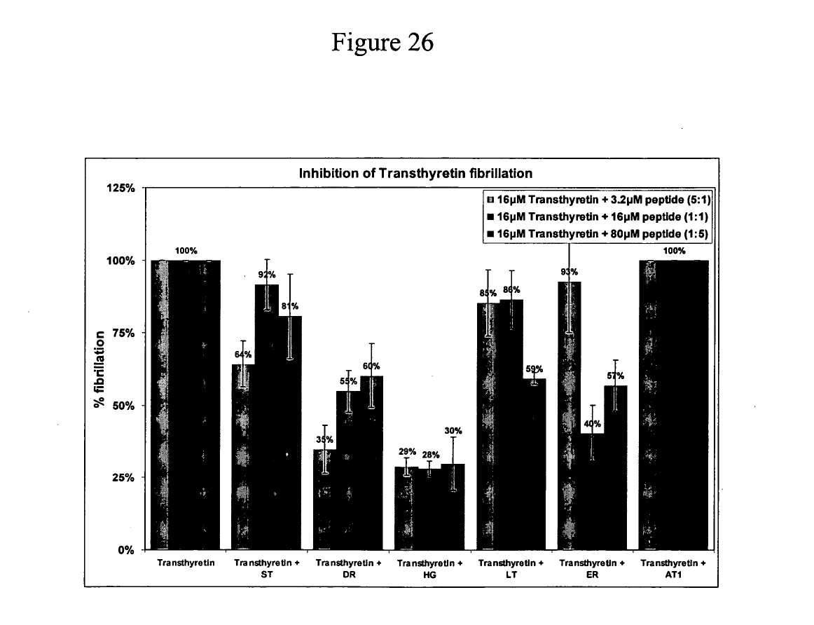

The effect of the five αB crystallin peptides ST, DR, HG, LT, and ER on TTR fibrillation was measured using the ThT fluorescence assay (FIG. 26). The formation and inhibition of TTR fibrils was qualitatively verified by fluorescence and bright-field microscopy. The concentration of the peptides was varied relative to TTR to establish a concentration curve. In addition, the effect of a control neuropeptide (AT1) with no TTR fibril inhibition activity was measured and compared to the effect of the αB crystallin peptides on TTR fibril formation. The ThT fluorescence of 16 μM TTR alone incubated at 37° C. for 4 hrs, increased from ˜15,000 to ˜45,000 RFU. Addition of 3.2 μM αB crystallin peptides, ST, DR, LT, and HG decreased the ThT fluorescence of TTR by 15-70%. In contrast, 3.2 μM ER had no effect on TTR fibril formation. Increasing the concentration of the ER peptide to 16 μM decreased the ThT fluorescence of TTR by 60%. The negative control peptide AT1 had no effect on TTR fibrillation even at a concentration as high as 160 μM. All five αB crystallin peptides inhibited TTR fibrillation at sub-stoichometric and stoichometric concentrations (3.2 μM and 16 μM).

FIG. 26 shows an effect of five αB crystallin peptides on transthyretin fibril formation. The Thioflavin T (ThT) fluorescence of TTR in the presence of varying concentrations of αB crystallin peptides and controls was measured. The addition of 3.2 μM ST, DR, HG and LT to 16 μM TTR resulted in a 15-71% decrease in ThT fluorescence. An equivalent amount (3.2 μM) of APS, NAP, and ER did not have any effect on ThT fluorescence. When the concentration of the peptides was increased to 16 μM (1:1::peptide:TTR), the αB crystallin peptides ST, DR, HG, LT and ER decreased the ThT fluorescence by 8-72%. When the concentration of the peptides was increased further to 80 μM (10:1::peptide:TTR), all of the five αB crystallin peptides ST, DR, HG, LT, and ER decreased the ThT fluorescence by 40-70%. The control peptide AT1 did not show a decrease in ThT fluorescence at any concentration including a 10:1 peptide:TTR ratio. The formation and inhibition of TTR fibrils was verified microscopically. The HG peptide was the most effective inhibitor of TTR fibril formation.

### Example 16

Effect of Synthesized αB Crystallin Peptides on β2-Microglobulin Fibril Formation

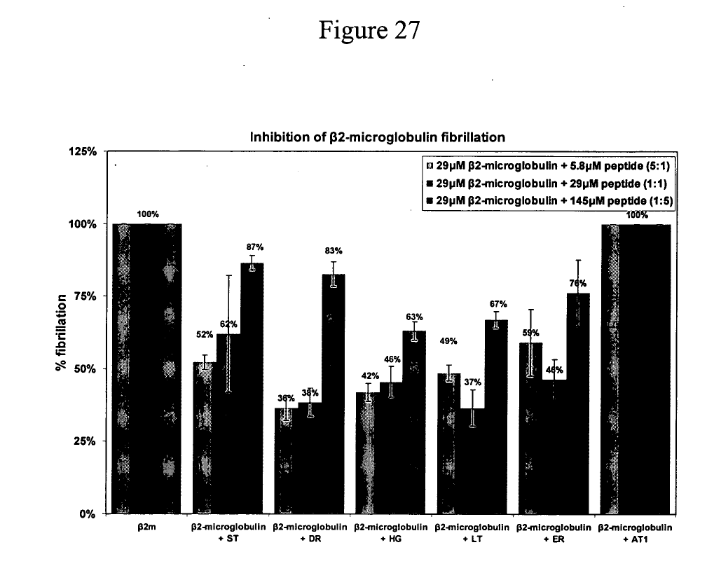

The effect of the five αB crystallin peptides ST, DR, HG, LT, and ER on (β2m fibrillation was measured using the ThT fluorescence assay (FIG. 27). The formation and inhibition of β2m fibrils was qualitatively verified by fluorescence and bright-field microscopy. The concentration of the peptides was varied relative to β2m to establish a concentration curve. In addition, the effect of a control neuropeptide (AT1) with no β2m fibril inhibition activity was measured and compared to the effect of the αB crystallin peptides on β2m fibrillation. The ThT fluorescence of 29 μM β2m alone incubated at 37° C. for 4 hrs, increased from ˜40,000 to ˜270,000 RFU. Addition of 5.8 μM αB crystallin peptides, ST, DR, LT, HG, and ER decreased the ThT fluorescence of β2m by 40-64%. When the concentration of the αB crystallin peptides was increased to 29 μM and 145 μM, no further decrease in ThT fluorescence was observed. The negative control peptide AT1 had no effect on β2m fibrillation even at a concentration as high as 290 μM. All five αB crystallin peptides inhibited β2m fibrillation at sub-stoichometric and stoichometric concentrations (5.8 μM and 29 μM).

FIG. 27 shows an effect of five αB crystallin peptides on β2-microglobulin fibril formation. The Thioflavin T (ThT) fluorescence of β2m in the presence of varying concentrations of αB crystallin peptides and controls was measured. The addition of 5.8 μM ST, DR, HG, LT or ER to 29 μM β2m resulted in a 40-65% decrease in ThT fluorescence. When the concentration of the peptides was increased to 29 μM (1:1::peptide:β2m), the αB crystallin peptides decreased the ThT fluorescence by 35-65%. When the concentration of the peptides was increased further to 145 μM (10:1::peptide:β2m), the αB crystallin peptides decreased the ThT fluorescence by 13-27%. The control peptide AT1 did not show a protective effect at any concentration including a 10:1 peptide:β2m ratio. The formation and inhibition of β2-microglobulin fibrils was verified microscopically. The HG and LT peptides were the most effective inhibitors of β2-microglobulin fibril formation.

### Example 17

Effect of Synthesized αB Crystallin Peptides on α-Synuclein Fibril Formation

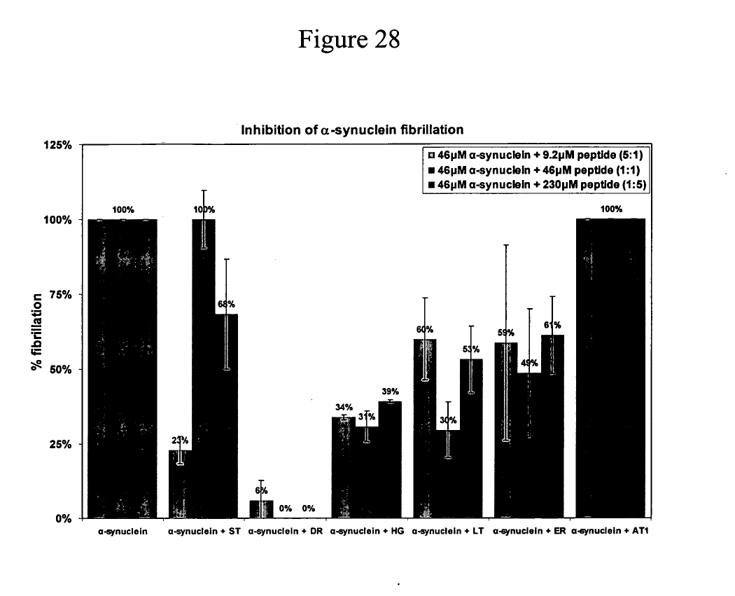

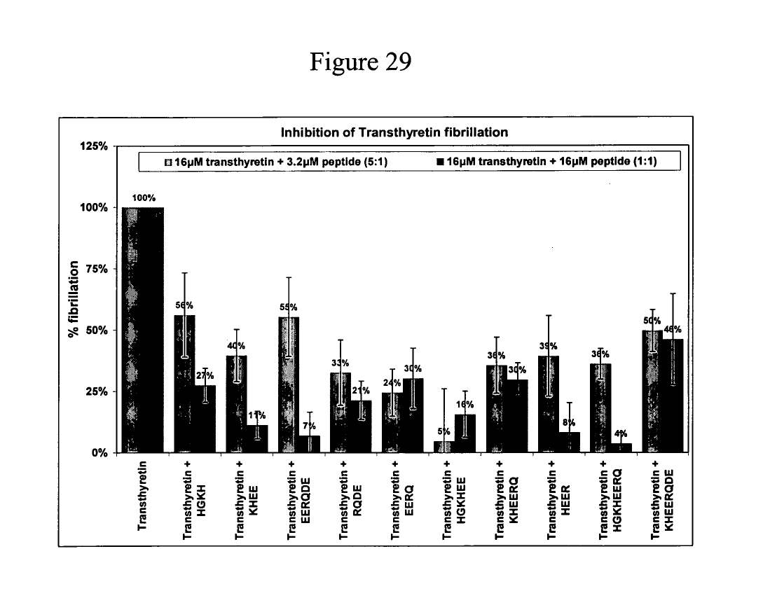

The effect of the five αB crystallin peptides, ST, DR, HG, LT, and ER on as synuclein fibrillation was measured using the ThT fluorescence assay (FIG. 28). The formation and inhibition of α-synuclein fibrils was qualitatively verified by fluorescence and bright-field microscopy. The concentration of the peptides was varied relative to α-synuclein to establish a concentration curve. In addition, the effect of a control neuropeptide (AT1) with no α-synuclein fibril inhibition activity was measured and compared to the effect of the αB crystallin peptides on α-synuclein fibrillation. The ThT fluorescence of 46 μM α-synuclein alone incubated at 37° C. for 96 hrs, increased from ˜9,500 to ˜16,000 RFU. Addition of 9.2 μM αB crystallin peptides ST, DR, HG, LT, and ER decreased the ThT fluorescence of α-synuclein by 32-100%. When the concentration of the αB crystallin peptides was increased to 46 μM, the ThT fluorescence of α-synuclein decreased with the αB crystallin peptides, DR, HG, LT, and ER, while the ThT fluorescence increased with the ST peptide. Increasing the concentration of the αB crystallin peptides further to a concentration of 230 μM did not decrease the ThT fluorescence of α-synuclein any further. The negative control peptide AT1 had no effect on α-synuclein fibrillation even at a concentration as high as 460 μM. All five αB crystallin peptides inhibited α-synuclein fibrillation at sub-stoichometric and stoichometric concentrations (9.2 μM and 46 μM).

FIG. 28 shows an effect of five αB crystallin peptides on α-synuclein fibril formation. The Thioflavin T (ThT) fluorescence of 46 μM α-synuclein in the presence of varying concentrations of αB crystallin peptides and controls was measured. The addition of 9.2 μM ST, DR, HG, LT, and ER to 46 μM α-synuclein resulted in a 32-100% decrease in ThT fluorescence after 96 hrs. When the concentration of the peptides was increased to 16 μM (1:1::peptide:α-synuclein), the αB crystallin peptides DR, HG, LT and ER decreased the ThT fluorescence by 51-100%, while the ST peptide had no effect on ThT fluorescence. When the concentration of the peptides was increased further to 80 μM (10:1::peptide:α-synuclein), the five αB crystallin peptides decreased the ThT fluorescence by 40-100%. The control peptide AT1 did not show a protective effect at any concentration including a 10:1 peptide:α-synuclein ratio. The formation and inhibition of β2-microglobulin fibrils was verified microscopically. The DR peptide was the most effective inhibitor of α-synuclein fibril formation.

### Example 18

Effect of Truncated HGKHEERQDE Peptides on Transthyretin Fibril Formation

The HGKHEERQDE was the most effective inhibitor of TTR fibrillation. To determine whether shorter peptide fragments of this peptide would be able to inhibit TTR fibrillation, ten truncated fragments of the HGKHEERQDE peptide HGKH, KHEE, EERQDE, RQDE, EERQ, HGKHEE, KHEERQ, HEER, HGKHEERQ, and KHEERQDE were synthesized and used in the TTR ThT assay (FIG. 27). The ten HGKHEERQDE derived peptides were HGKH, KHEE, EERQDE, RQDE, EERQ, HGKHEE, KHEERQ, HEER, HGKHEERQ, and KHEERQDE. 3.2 μM αB crystallin peptides, HGKH, KHEE, EERQDE, RQDE, EERQ, HGKHEE, KHEERQ, HEER, and HGKHEERQ decreased the ThT fluorescence of TTR by 44-95%. Increasing the concentration of the peptides to 16 μM resulted in increased inhibition of TTR fibrillation by seven of the ten peptides. All ten HGKHEERQDE derived peptides inhibited TTR fibrillation at sub-stoichometric and stoichometric concentrations (3.2 μM and 16 μM).

FIG. 27 shows an effect of HG derived peptides on TTR fibrillation. The Thioflavin T (ThT) fluorescence of TTR in the presence of varying concentrations of truncated fragments of the αB crystallin HGKHEERQDE peptide was measured. The addition of 3.2 μM HGKH, KHEE, EERQDE, RQDE, EERQ, HGKHEE, KHEERQ, HEER, HGKHEERQ, and KHEERQDE resulted in a 44-95% decrease in ThT fluorescence after 48 hrs. When the concentration of the peptides was increased to 16 μM (1:1::peptide:TTR), the peptides HGKH, KHEE, EERQDE, RQDE, KHEERQ, HEER, and HGKHEERQ decreased the ThT fluorescence by 54-96%. Increasing the concentration of the peptides, EERQ and HGKHEE decreased their effect on TTR fibril inhibition by 5-10%. EERQDE, HEER, and HGKHEERQ were the most effective inhibitors of TTR fibrils and achieved almost 100% inhibition at a 1:1 ratio.

### Example 19

Effect of Truncated HGKHEERQDE Peptides on β2-Microglobulin Fibril Formation

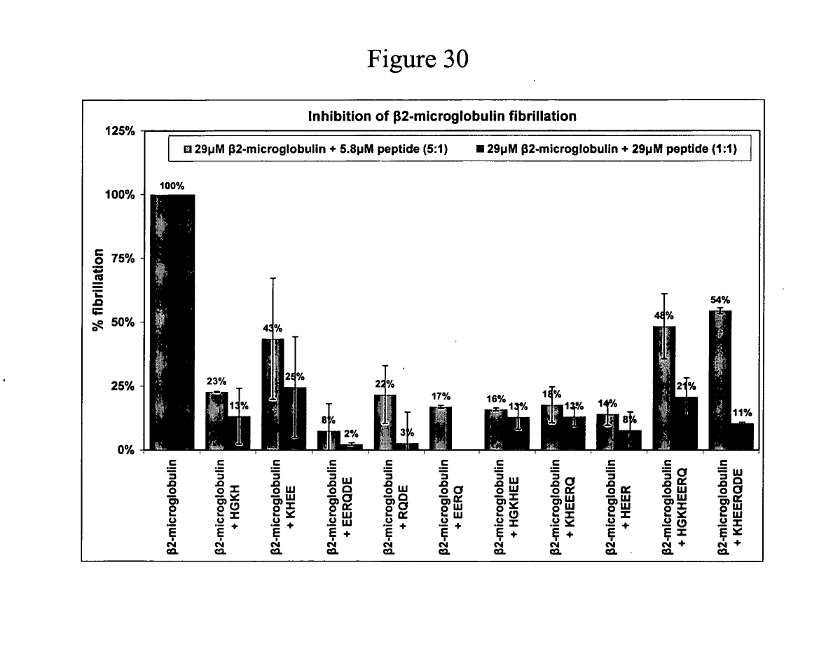

The HGKHEERQDE was the most effective inhibitor of β2m fibrillation. To determine whether shorter peptide fragments of this peptide would be able to inhibit β2m fibrillation, ten truncated fragments of the HGKHEERQDE peptide HGKH, KHEE, EERQDE, RQDE, EERQ, HGKHEE, KHEERQ, HEER, HGKHEERQ, and KHEERQDE were synthesized and used in the β2m ThT assay (FIG. 30). 5.8 μM HGKHEERQDE derived peptides, HGKH, KHEE, EERQDE, RQDE, EERQ, HGKHEE, KHEERQ, HEER, and HGKHEERQ decreased the ThT fluorescence of β2m by 44-100%. Increasing the concentration of the peptides to 29 μM resulted in increased inhibition of β2m fibrillation by all ten peptides. All ten HGKHEERQDE derived peptides inhibited β2m fibrillation at sub-stoichometric and stoichometric concentrations (5.8 μM and 29 μM).

FIG. 30 shows an effect of HG derived peptides on β2m fibrillation. The Thioflavin T (ThT) fluorescence of β2m in the presence of varying concentrations of truncated fragments of the αB crystallin HGKHEERQDE peptide was measured. The addition of 5.8 μM HGKH, KHEE, EERQDE, RQDE, EERQ, HGKHEE, KHEERQ, HEER, HGKHEERQ, and KHEERQDE to 29 μM β2m resulted in a 44-100% decrease in ThT fluorescence after 48 hrs. When the concentration of the peptides was increased to 29 μM (1:1::peptide:β2m), the αB crystallin peptides decreased the ThT fluorescence by 75-98%. The EERQDE, RQDE, and EERQ peptides were the most effective inhibitors of β2-microglobulin fibril formation and achieved almost 100% inhibition at a 1:1 ratio.

### Example 20

Effect of Truncated HGKHEERQDE Peptides on α-Synuclein Fibril Formation

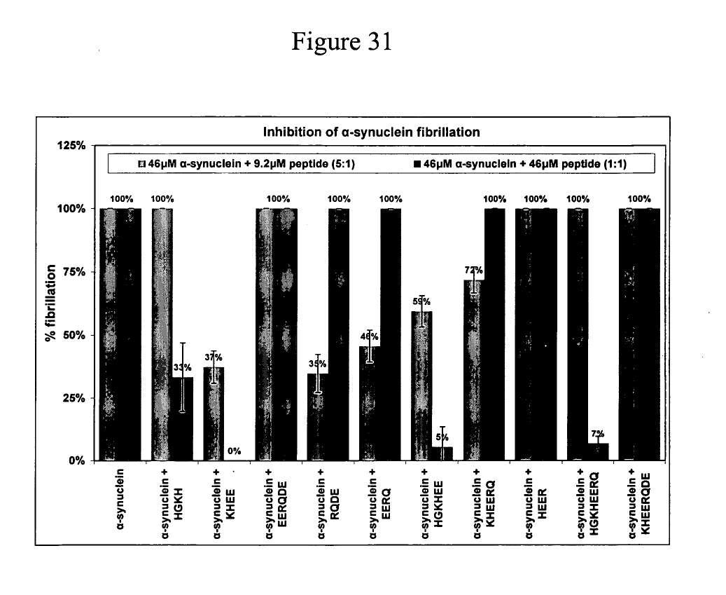

The HGKHEERQDE was the most effective inhibitor of α-synuclein. To determine whether shorter peptide fragments of this peptide would be able to inhibit α-synuclein fibrillation, ten truncated fragments of the HGKHEERQDE peptide HGKH, KHEE, EERQDE, RQDE, EERQ, HGKHEE, KHEERQ, HEER, HGKHEERQ, and KHEERQDE were synthesized and used in the α-synuclein ThT assay (FIG. 31). 5.8 μM HGKHEERQDE derived peptides, KHEE, RQDE, EERQ, HGKHEE, and KHEERQ decreased the ThT fluorescence of α-synuclein by 28-65%. Increasing the concentration of the peptides to 29 μM resulted in a 67-93% decrease in α-synuclein fibrillation by HGKH and HGKHEERQ. Seven of ten HGKHEERQDE derived peptides inhibited α-synuclein fibrillation at sub-stoichometric or stoichometric concentrations (5.8 μM and 29 μM).

FIG. 31 shows an effect of HG derived peptides on α-synuclein fibrillation. The Thioflavin T (ThT) fluorescence of Aβ in the presence of varying concentrations of truncated fragments of the αB crystallin HGKHEERQDE peptide was measured. The addition of 3.2 μM HGKH, KHEE, EERQDE, RQDE, EERQ, HGKHEE, KHEERQ, HEER, HGKHEERQ, and KHEERQDE resulted in a 44-95% decrease in ThT fluorescence after 48 hrs. When the concentration of the peptides was increased to 16 μM (1:1::peptide:TTR), the peptides HGKH, KHEE, EERQDE, RQDE, KHEERQ, HEER, and HGKHEERQ decreased the ThT fluorescence by 54-96%. Increasing the concentration of the peptides, EERQ and HGKHEE decreased their effect on TTR fibril inhibition by 5-10%. EERQDE, HEER, and HGKHEERQ were the most effective inhibitors of TTR fibrils and achieved almost 100% inhibition at a 1:1 ratio.

### EXAMPLE 21

Peptides of Molecular Chaperone αB Crystallin Inhibit Fibrillation Of Amyloidogenic Proteins

The present report has identified and evaluated fifteen interactive sequences from the molecular chaperone αB crystallin for their ability to inhibit fibrillation of a number of amyloidogenic proteins including Aβ, transthyretin, β2-microglobulin, and α-synuclein.

Initially five previously described chaperone sequences of human αB crystallin were evaluated for their ability to inhibit the fibrillation of the four amyloidogenic proteins, Aβ, transthyretin, β2-microglobulin, and α-synuclein. In vitro fibril assays indicated that at stoichometric and sub-stoichometric concentrations, the αB crystallin peptides inhibited the fibrillation of these amyloidogenic proteins as measured by bright-field microscopy and Thioflavin T fluorescence. At sub-stoichometric concentrations (peptide to substrate concentration of 1:5), the αB crystallin peptides, STSLSPFYLRPPSFLRAP and HGKHEERQDE achieved complete inhibition (100%) of Aβ fibrillation. At equivalent concentrations, the two molecules, Tramiprosate and NAP that are currently in human clinical trials for amyloidoses achieved only partial inhibition (˜38%) of Aβ fibrillation. At higher concentrations, the αB crystallin peptides DR, LT, and ER achieved 60-100% inhibition of Aβ fibrillation. The inhibition of Aβ fibrillation by the DR peptide was consistent with a previous report in which an αA crystallin peptide homologous to the DR peptide was observed to inhibit Aβ fibrillation and toxicity. Santhoshkumar and Sharma, Mol Cell Biochem 267:147-55, 2004.

Similarly, in experiments with transthyretin, β2-microglobulin, and α-synuclein, the αB crystallin peptides achieved 15-100% inhibition of fibrillation at sub-stoichometric concentrations, while the control peptide AT1 had no effect on fibrillation at the concentrations tested (1:10, 1:5, 1:1, 5:1, and 10:1). The HGKHEERQDE peptide was the most effective inhibitor of fibrillation. Ten truncated fragments of the HGKHEERQDE peptide varying in length from 4-8 amino acids achieved almost complete inhibition of TTR and β2m fibrillation at stoichometric and sub-stoichometric concentrations. The ability of the αB crystallin peptides to inhibit fibrillation of all six amyloidogenic proteins studied indicated a common mechanism of action for these peptides. The 3D structures of sHSPs including αB crystallin are similar to transthyretin and β2-microglobulin in that they have an immunoglobulin-like fold consisting of 6-100 strands arranged in two anti-parallel β sheets forming a compact β sandwich. In the 3D structure of αB crystallin, the peptides, DR and LT formed β strands, the HG and ER peptides have flexible secondary structure, and the ST peptide has partial helical structure (42, 43, 53-55). Ghosh et al., Biochemistry 44:14854-69, 2005; Ghosh and Clark, Protein Sci 14:684-95, 2005; Ghosh et al, Cell Stress Chaperones 11:187-97, 2006; Guruprasad and Kumari, Int J Biol Macromol 33:107, 2003; Koteiche and McHaourab, J Mol Biol 294:561-77, 1999. The similarity in 3D structure of αB crystallin, the archetype of sHSPs, and the two amyloidogenic proteins, transthyretin and β2-microglobulin suggested a structural basis for the inhibition of fibrillation of these two proteins.

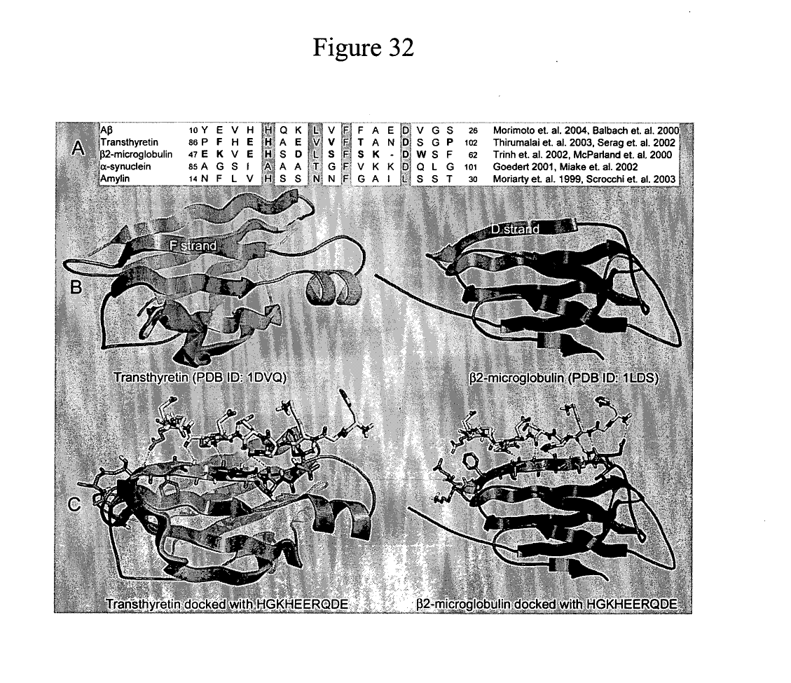

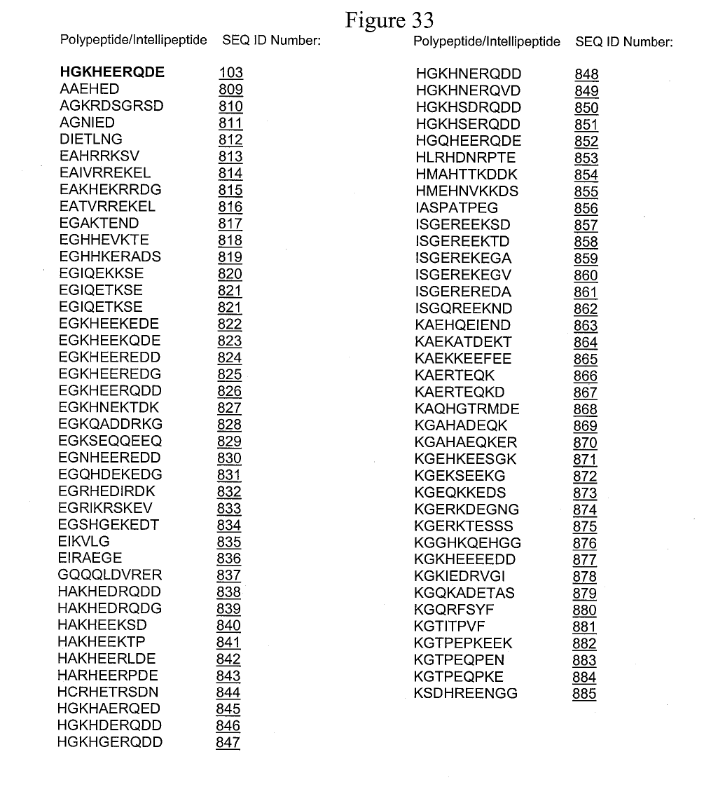

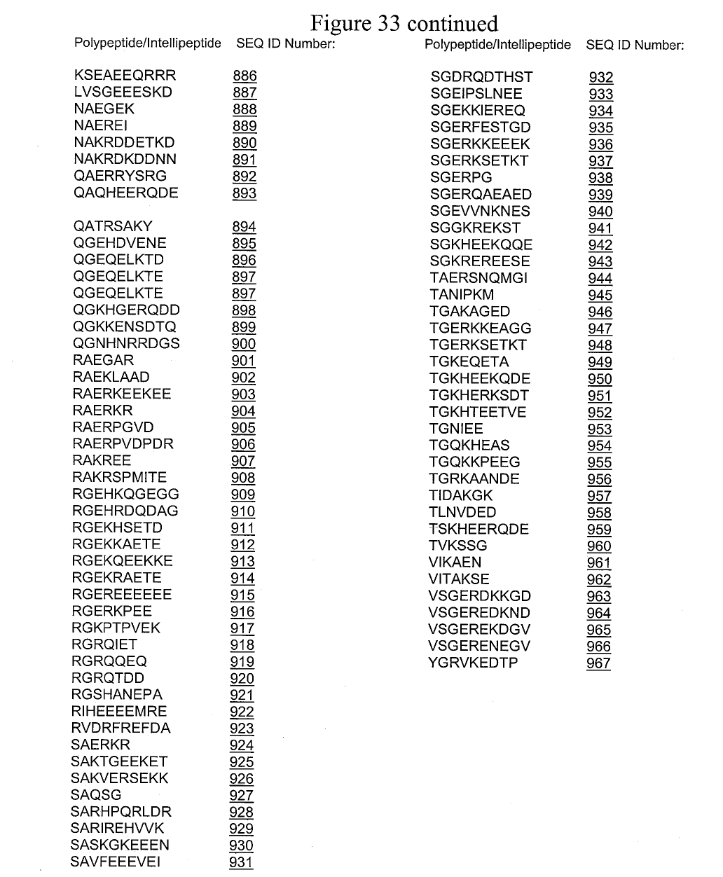

Sequence analysis of the amyloidogenic regions of Aβ, transthyretin, β2-microglobulin, and α-synuclein (Morimoto et al., J Biol Chem 279:52781-8, 2004; Balbach et al., Biochemistry 39:13748-59, 2000; Thirumalai et al., Curr Opin Struct Biol 13:146-59, 2003; Serag et al., Nat Struct Biol 9:734-9, 2002; Trinh et al., Proc Natl Acad Sci USA 99:9771-6, 2002; McParland et al., Biochemistry 39:8735-46, 2000; Goedert, M., Nat Rev Neurosci 2:492-501, 2001; Miake et al., J Biol Chem 277:19213-9, 2002) and structural analysis of the crystal structures of β2-microglobulin and transthyretin indicated the presence of a common amyloidogenic region in three of the four proteins (Aβ, β2-microglobulin, and transthyretin) that had a propensity to form a β strand secondary structure (FIG. 32A). In the crystal structure of human transthyretin, this region forms the F strand, while in the crystal structure of β2-microglobulin, this region forms the D strand (FIG. 32B). To determine the mechanism by which the αB crystallin peptides interact and inhibit the fibrillation of transthyretin and β2-microglobulin, the αB crystallin peptides were individually docked with the crystal structures of human transthyretin and β2-microglobulin. It is important to note that the active sites for docking the αB crystallin peptides onto the 3D structures of transthyretin and β2-microglobulin were not specified during the docking run, only the 3D co-ordinates for the αB crystallin peptides and crystal structures of transthyretin and β2-microglobulin were provided to the ClusPro docking program. The transthyretin and β2-microglobulin docked models, indicated that the αB crystallin peptides that inhibited fibrillation had a propensity to interact with the amyloidogenic β strands of the transthyretin (F strand) and β2-microglobulin (D strand) (FIG. 32C). In the transthyretin-docked models, the αB crystallin peptides, DR and HG that showed strong inhibition of TTR fibrils interacted directly with amyloidogenic F strand of transthyretin. Similarly, the ER peptide that modestly inhibited TTR fibrillation interacted partially with the F strand of transthyretin. In contrast, the ST and LT peptides that partially inhibited TTR fibril formation did not interact with F strand of TTR. In the β2-microglobulin-docked models, the αB crystallin peptides ST, DR, HG, and ER interacted directly with the amyloidogenic D strand, while the LT peptide interacted with a different amyloidogenic region of β2-microglobulin. Based on the effect of the αB crystallin peptides on fibril formation, sequence and structure analysis and protein-peptide docking, we hypothesize that αB crystallin peptides inhibit fibrillation by interacting directly with the amyloidogenic regions of proteins that have a propensity to unfold and form toxic fibrils.

FIG. 32 shows a hypothetical mechanism by which αB crystallin peptides inhibit the fibrillation of amyloidogenic proteins. A) Sequence alignment of the amyloidogenic regions of amyloid-β (Aβ), transthyretin, β2-microglobulin, α-synuclein, and amylin. The amyloidogenic regions of Aβ, transthyretin, β2-microglobulin, and α-synuclein identified previously (Morimoto et al., J Biol Chem 279:52781-8, 2004; Balbach et al., Biochemistry 39:13748-59, 2000; Thirumalai et al., Curr Opin Struct Biol 13:146-59, 2003; Serag et al., Nat Struct Biol 9:734-9, 2002; Trinh et al., Proc Natl Acad Sci USA 99:9771-6, 2002; McParland et al., Biochemistry 39:8735-46, 2000; Goedert, M., Nat Rev Neurosci 2:492-501, 2001; Miake et al., J Biol Chem 277:19213-9, 2002) were aligned using DOTTER. Sonnhammer and Durbin, Gene 167:GC1-10, 1995. Conserved residues are highlighted with grey backgrounds and surface exposed residues in the crystal structures of transthyretin and β2-microglobulin are in BOLD. B) Crystal structures of the monomeric subunits of transthyretin and β2-microglobulin. The amyloidogenic regions in both proteins are shown in blue. In transthyretin, the amyloidogenic region forms the F strand (Thirumalai et al., Curr Opin Struct Biol 13:146-59, 2003; Serag et al., Nat Struct Biol 9:734-9, 2002), while in β2-microglobulin, the amyloidogenic region forms the D strand (Trinh et al., Proc Natl Acad Sci USA 99:9771-6, 2002; McParland et al., Biochemistry 39:8735-46, 2000) C) Computed models of the αB crystallin peptide HGKHEERQDE docked with the monomers of transthyretin and β2-microglobulin. The HG peptide was docked with the crystal structures of monomeric human transthyretin and β2-microglubulin using the ClusPro protein-protein docking program. Comeau et al., Nucleic Acids Res 32:W96-9, 2004; Comeau et al., Bioinformatics 20:45-50, 2004. The HG peptide interacts with residues of the F stand and the D strand of transthyretin and β2-microglobulin respectively. Similar interactions were observed when the remaining αB crystallin peptides were docked with the crystal structures of transthyretin and β2-microglobulin respectively. The interaction between the HG peptide and transthyretin/β2-microglobulin appears to be mediated predominantly by salt bridges.

The results presented in this report validate experimentally the strategy of identifying and characterizing novel peptide chaperones that modify the monomer→oligomer→fibril pathway of amyloidogenic proteins, which is one of the main disease-causing pathways in various amyloidoses including Alzheimer's disease, senile systemic amyloidosis, hemodialysis-related amyloidosis, and Parkinson's disease. The αB crystallin peptides identified in this report selectively inhibit fibrillation at low concentrations and are excellent building blocks for the rational design of peptidic and peptidomimetic inhibitors that target select oligomerization and fibrillation interfaces of specific amyloidogenic proteins.

### Example 22

Materials and Methods

Materials. The αB crystallin peptides ₇₃DRFSVNLDVKHFS₈₅ (DR) and ₁₀₁HGKHEERQDE₁₁₀ (HG) were synthesized by Advanced ChemTech. The remaining three αB crystallin peptides, ₄₁STSLSPFYLRPPSFLRAP₅₈ (ST), ₁₃₁LTITSSLSSDGV₁₄₂ (LT) and ₁₅₆ERTIPITRE₁₆₄ (ER), were synthesized by Genscript. The peptides, NAPVSIPQ (NAP) and DRVYIHPFHL (Angiotensin 1, AT1) were purchased from Genscript. Aβ₁₋₄₂ (American Peptide), transthyretin (Sigma-Aldrich), β2-microglobulin (MP Biomedical), α-synuclein (rPeptide) were also procured from external vendors as listed above. Tramiprosate (3-aminopropanesulfonic acid, APS) was purchased from Sigma-Aldrich. The peptides, HGKH, KHEE, EERQDE, RQDE, EERQ, HGKHEE, KHEERQ, HEER, HGKHEERQ, and KHEERQDE were synthesized and procured from Genscript.

Thioflavin T (ThT) Assays. The effect of the five αB crystallin peptides (ST, DR, HG, LT, and ER) on Aβ₁₋₄₂ fibril formation was measured using the ThT fluorescence assay (44-47) and compared with two known inhibitors of Aβ₁₋₄₂ fibril formation (Tramiprosate and NAP)(34, 36) and a negative control neuropeptide Angiotensin 1 (AT1). Thioflavin T is a fluorescent dye that has a high affinity for amyloid fibrils and becomes fluorescent (excitation λ=450 nm, emission λ=486 nm) when bound to fibrils. For the Aβ₁₋₄₂ assays, peptides and APS were dissolved to 2 mM in 0.25% DMSO and diluted accordingly, while Aβ₁₋₄₂ was dissolved in 20 mM Tris-Cl, pH 7.40, and 100 mM NaCl. For the 5:1::inhibitor:Aβ₁₋₄₂ assay, 10 μL of 450 μM peptide or APS was mixed with 10 μL of 90 μM Aβ₁₋₄₂), 7 μL 20 mM Tris-Cl, 100 mM NaCl, pH 7.4 and 3 μL of 1 mM ThT. For the 1:1::inhibitor:Aβ₁₋₄₂ assay, 10 μL of 90 μM peptide or APS was mixed with 10 μL of 90 μM Aβ(1-42), 7 μL 20 mM Tris-Cl, 100 mM NaCl, pH 7.4 and 3 μL of 1 mM ThT. For the 1:5::inhibitor:Aβ₁₋₄₂ assay, 10 μL of 18 μM peptide or APS was mixed with 10 μL of 90 μM Aβ(1-42), 7 μL 20 mM Tris-Cl, 100 mM NaCl, pH 7.4 and 3 μL of 1 mM ThT. ThT fluorescence in relative fluorescence units (RFU) was measured using a Perkin Elmer Victor³ V plate reader. Assays were performed in 384-well fluorescence plates in duplicates or triplicates. Samples were incubated at 37° C. for a total of 48 hours. ThT fluorescence was measured at 0, 24, and 48 hours. The effect of the peptides and APS was calculated as percentage fibril formation relative to Aβ₁₋₄₂ alone (100%) at the end of 48 hours.

Transthyretin fibrillation ThT assays were performed as described previously. Hurshman et al., Biochemistry 43:7365-81, 2004; O'Nuallain and Wetzel, Proc Natl Acad Sci USA 99:1485-90, 2002. Peptides were dissolved to 2 mM in 0.25% DMSO and diluted accordingly, while TTR was dissolved in 200 mM KCl, 2 mM EDTA, 5 mM PBS, 200 mM sodium acetate, pH 4.4. For the 5::peptide:TTR assay, 10 μL of 160 μM peptide was mixed with 10 μL of 32 μM TTR, and 1 μL of 1 mM ThT. For the 1:1::peptide:TTR assay, 10 μL of 32 μM peptide was mixed with 10 μL of 32 μM TTR and 1 μL of 1 mM ThT. For the 1:5::peptide:TTR assay, 10 μL of 6.4 μM peptide was mixed with 10 μL of 32 μM TTR and 1 μL of 1 mM ThT. ThT fluorescence was measured using a Perkin Elmer Victor³ V plate reader. Assays were performed in 384-well fluorescence plates in duplicates or triplicates. Samples were incubated at 37° C. ThT fluorescence was measured continuously for 150 minutes. The effect of the peptides and APS was calculated as percentage fibril formation relative to TTR alone (100%) at the end of 150 minutes.

β2-microglobulin fibrillation ThT assays were performed as described previously. Raman et al., Biochem J 392:573-81, 2005; Ivanova et al., Proc Natl Acad Sci USA 101:10584-9, 2004. Peptides were dissolved to 2 mM in 0.25% DMSO and diluted accordingly, while β2-microglobulin was dissolved in 25 mM sodium acetate, 25 mM sodium phosphate, pH 2.4. For the 5:1::peptide:β2-microglobulin assay, 10 μL of 430 μM peptide was mixed with 10 μL of 86 μM β2-microglobulin, 7 μl of 25 mM sodium acetate, 25 mM sodium phosphate, pH 2.4 and 3 μL of 1 mM ThT. For the 1:1::peptide:β2-microglobulin assay, 10 μL of 86 μM peptide was mixed with 10 μL of 86 μM (2-microglobulin, 7 μl of 25 mM sodium acetate, 25 mM sodium phosphate, pH 2.4 and 3 μL of 1 mM ThT. For the 1:5::peptide:β2-microglobulin assay, 10 μL of 17.2 μM peptide was mixed with 10 μL of 86 μM β2-microglobulin, 7 μl of 25 mM sodium acetate, 25 mM sodium phosphate, pH 2.4 and 3 μL of 1 mM ThT. ThT fluorescence was measured using a Perkin Elmer Victor³ V plate reader. Assays were performed in 384-well fluorescence plates in duplicates or triplicates. Samples were incubated at 37° C. ThT fluorescence was measured continuously for 180 minutes. The effect of the peptides and APS was calculated as percentage fibril formation relative to β2-microglobulin alone (100%) at the end of 180 minutes.

α-synuclein fibrillation ThT assays were performed as described previously. Zhu et al., J Biol Chem 279:26846-57, 2004; Dedmon et al., J Biol Chem 280:14733-40, 2005. Peptides were dissolved to 2 mM in 0.25% DMSO and diluted accordingly, while α-synuclein was dissolved in 20 mM Tris-Cl, 100 mM NaCl, pH 7.4. For the 5:1::peptide: α-synuclein assay, 5 μL of 700 μM peptide or APS was mixed with 5 μL of 140 μM α-synuclein, 17 μt of 20 mM Tris-Cl, 100 mM NaCl, pH 7.4 and 3 μL of 1 mM ThT. For the 1:1::peptide:α-synuclein assay, 5 μL of 140 μM peptide was mixed with 5 μL of 140 μM α-synuclein, 17 μl of 20 mM Tris-Cl, 100 mM NaCl, pH 7.4 and 3 μL of 1 mM ThT. For the 1:5::peptide:α-synuclein assay, 5 μL of 28 μM peptide was mixed with 5 μL of 140 μM α-synuclein, and 7 μl of 20 mM Tris-Cl, 100 mM NaCl, pH 7.4 and 3/L of 1 mM ThT. ThT fluorescence was measured using a Perkin Elmer Victor³ V plate reader. Assays were performed in 384-well fluorescence plates in duplicates or triplicates. Samples were incubated at 37° C. for a total of 96 hours. ThT fluorescence was measured at 0, 24, 48, and 96 hours. The effect of the peptides and APS was calculated as percentage fibril formation relative to α-synuclein alone (100%) at the end of 96 hours.

This description of the invention, will enable those skilled in the art to perform within a wide range of equivalent parameters, concentrations, and conditions without departing from the spirit and scope of the invention and without due experimentation results that are presented here.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

1. A polypeptide X₁-HGKHEERQDE-X₂ (SEQ ID NO:85), or a functional variant or mimetic thereof,
wherein each X₁ and X₂ independently of one another represents any amino acid sequence of n amino acids, n varying from 0 to 50, and n being identical or different in X₁ and X₂.

2. The polypeptide of claim 1 wherein the functional variant or mimetic is a conservative amino acid substitution or peptide mimetic substitution.

3. The polypeptide of claim 1 wherein the functional variant has about 70% or greater amino acid sequence identity to X₁-HGKHEERQDE-X₂ (SEQ ID NO:85).

4. The polypeptide of claim 1 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

5. The polypeptide of claim 1 wherein the functional variant has about 70% or greater amino acid sequence identity to X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

6. A polypeptide X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95), or a functional variant or mimetic thereof,
wherein each X₁ and X₂ independently of one another represents any amino acid sequence of n amino acids, n varying from 0 to 50, and n being identical or different in X₁ and X₂.

7. The polypeptide of claim 6 wherein the functional variant has about 70% or greater amino acid sequence identity to X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

8. A method for treating a protein conformation disease in a mammalian subject comprising administering a polypeptide to the subject in need thereof, wherein the polypeptide is X₁-HGKHEERQDE-X₂ (SEQ ID NO:85), or a functional variant or mimetic thereof, wherein each X₁ and X₂ independently of one another represents any amino acid sequence of n amino acids, n varying from 0 to 50, and n being identical or different in X₁ and X₂.

9. The method of claim 6 wherein the functional variant or mimetic comprises a conservative amino acid substitution or peptide mimetic substitution.

10. The method of claim 6 wherein the functional variant comprises about 70% or greater amino acid sequence identity to X₁-HGKHEERQDE-X₂ (SEQ ID NO:85).

11. The method of claim 6 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

12. The method of claim 6 wherein the functional variant has about 70% or greater amino acid sequence identity to X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

13. The method of claim 6 wherein the disease is Alexander's disease, Alzheimer's disease, Creutzfeld-Jakob disease, Parkinson's disease, senile systemic amyloidosis, hemodialysis-related amyloidosis, Huntington's disease, cataract, retinitis pigmentosa, prion disease, or mad cow disease.

14. The method of claim 6 wherein the disease is age-related myopathy.

15. The method of claim 6 wherein the disease is cardiac ischemia.

16. A method for stabilizing a protein comprising contacting the protein with a polypeptide X₁-HGKHEERQDE-X₂ (SEQ ID NO:85), or a functional variant or mimetic thereof, wherein each X₁ and X₂ independently of one another represents any amino acid sequence of n amino acids, n varying from 0 to 50, and n being identical or different in X₁ and X₂.

17. The method of claim 14 wherein the functional variant has about 70% or greater amino acid sequence identity to X₁-HGKHEERQDE-X₂ (SEQ ID NO:85).

18. The method of claim 14 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

19. The method of claim 14 wherein the functional variant has about 70% or greater amino acid sequence identity to X₁-HGKH-X₂ (SEQ ID NO:86), X₁-KHEE-X₂ (SEQ ID NO: 87), X₁-EERQDE-X₂ (SEQ ID NO: 88), X₁-RQDE-X₂ (SEQ ID NO: 89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

20. The method of claim 14 wherein the protein is a therapeutic protein.

21. The method of claim 18 wherein the therapeutic protein is a vaccine, insulin, growth factor, or antibody.

22. The method of claim 18, further comprising increasing the stability of the therapeutic protein to treat a disease state in a mammalian subject.

23. The method of claim 14 wherein the protein is a recombinantly-produced protein.

24. The method of claim 14, further comprising inhibiting protein misfolding or reducing protein aggregation.

25. The method of claim 14, further comprising restoring correct or native folding to the protein.

26. The method of claim 14 wherein the functional variant or mimetic comprises a conservative amino acid substitution or peptide mimetic substitution.

27. The method of claim 14 wherein the functional variant comprises about 70% or greater amino acid sequence identity to X₁-HGKHEERQDE-X₂ (SEQ ID NO:85).

28. A method for diagnosing a protein conformation disease in a mammalian subject comprising:
contacting a tissue sample from the mammalian subject with a polypeptide X₁-HGKHEERQDE-X₂ (SEQ ID NO:85), or a functional variant or mimetic thereof, wherein each X₁ and X₂ independently of one another represents any amino acid sequence of n amino acids, n varying from 0 to 50, and n being identical or different in X₁ and X₂,
detecting binding of a misfolded protein, an unfolded protein, or an aggregated protein to the polypeptide, and
determining a presence or absence of the disease in the mammalian subject.

29. The method of claim 26 wherein the functional variant has about 70% or greater amino acid sequence identity to X₁-HGKHEERQDE-X₂ (SEQ ID NO:85).

30. The method of claim 26 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

31. The method of claim 26 wherein the functional variant has about 70% or greater amino acid sequence identity to X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

32. The method of claim 26 wherein the presence or absence of the disease is detected by a stage of misfolding, unfolding, or aggregation of the protein.

33. The method of claim 26 wherein the protein conformation disease is Alexander's disease, Alzheimer's disease, Creutzfeld-Jakob disease, Parkinson's disease, senile systemic amyloidosis, hemodialysis-related amyloidosis, Huntington's disease, cataract, retinitis pigmentosa, prion disease, or mad cow disease.

34. The method of claim 26 wherein the disease is age-related myopathy.

35. The method of claim 26 wherein the disease is cardiac ischemia.

36. The method of claim 26 wherein the functional variant or mimetic comprises a conservative amino acid substitution or peptide mimetic substitution.

37. The method of claim 26 wherein the functional variant comprises about 70% or greater amino acid sequence identity to X₁-HGKHEERQDE-X₂ (SEQ ID NO:85).

38. An in vitro method of screening for a modulator of protein misfolding, protein unfolding, or protein aggregation activity comprising:
contacting a target protein with a polypeptide X₁-HGKHEERQDE-X₂ (SEQ ID NO:85), or a functional variant or mimetic thereof, wherein each X₁ and X₂ independently of one another represents any amino acid sequence of n amino acids, n varying from 0 to 50, and n being identical or different in X₁ and X₂,
and detecting a decrease in the amount of protein aggregation activity; thereby identifying the polypeptide as a modulator of protein misfolding, protein unfolding, or protein aggregation activity.

39. The method of claim 36 wherein the functional variant has about 70% or greater amino acid sequence identity to X₁-HGKHEERQDE-X₂ (SEQ ID NO:85).

40. The method of claim 36 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

41. The method of claim 36 wherein the functional variant has about 70% or greater amino acid sequence identity to X₁-HGKH-X₂ (SEQ ID NO:86), X₁-KHEE-X₂ (SEQ ID NO: 87), X₁-EERQDE-X₂ (SEQ ID NO: 88), X₁-RQDE-X₂ (SEQ ID NO: 89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

42. The method of claim 36 wherein the target protein is amyloid-beta, beta/gamma crystallins, actin, desmin, vimentin, insulin, citrate synthase, alcohol dehydrogenase, glial fibrillary acidic protein, alpha-lactalbumin, fibroblast growth factor, insulin-like growth factor, transforming growth factor-beta, nerve growth factor-beta, epidermal growth factor, vascular endothelial growth factor, beta-catenin, tumor necrosis factor-alpha, Bcl-2, Bcl-X1 or caspase.

43. The method of claim 36 wherein the functional variant or mimetic comprises a conservative amino acid substitution or peptide mimetic substitution.

44. The method of claim 36 wherein the functional variant comprises about 70% or greater amino acid sequence identity to X₁-HGKHEERQDE-X₂ (SEQ ID NO:85).

45. An in vivo method of screening for a modulator of protein misfolding, protein unfolding, or protein aggregation activity comprising:
contacting a cell or cell line expressing a target protein with a test compound encoding a polypeptide X₁-HGKHEERQDE-X₂ (SEQ ID NO:85), or a functional variant or mimetic thereof, wherein each X₁ and X₂ independently of one another represents any amino acid sequence of n amino acids, n varying from 0 to 50, and n being identical or different in X₁ and X₂,
and detecting a decrease in the amount of protein aggregation activity in the cell line, thereby identifying the polypeptide as a modulator of protein misfolding, protein unfolding, or protein aggregation activity.

46. The method of claim 43 wherein the functional variant has about 70% or greater amino acid sequence identity to X₁-HGKHEERQDE-X₂ (SEQ ID NO:85).

47. The method of claim 43 wherein the functional variant is X₁-HGKH-X₂ (SEQ ID NO:86), X₁-KHEE-X₂ (SEQ ID NO:87), X₁-EERQDE-X₂ (SEQ ID NO:88), X₁-RQDE-X₂ (SEQ ID NO:89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

48. The method of claim 43 wherein the functional variant has about 70% or greater amino acid sequence identity to X₁-HGKH-X₂ (SEQ ID NO:86), X₁-KHEE-X₂ (SEQ ID NO: 87), X₁-EERQDE-X₂ (SEQ ID NO: 88), X₁-RQDE-X₂ (SEQ ID NO: 89), X₁-EERQ-X₂ (SEQ ID NO:90), X₁-HGKHEE-X₂ (SEQ ID NO:91), X₁-KHEERQ-X₂ (SEQ ID NO:92), X₁-HEER-X₂ (SEQ ID NO:93), X₁-HGKHEERQ-X₂ (SEQ ID NO:94), or X₁-KHEERQDE-X₂ (SEQ ID NO:95).

49. The method of claim 43 wherein the target protein is amyloid-beta, beta/gamma crystallins, actin, desmin, vimentin, insulin, citrate synthase, alcohol dehydrogenase, glial fibrillary acidic protein, alpha-lactalbumin, fibroblast growth factor, insulin-like growth factor, transforming growth factor-beta, nerve growth factor-beta, epidermal growth factor, vascular endothelial growth factor, beta-catenin, tumor necrosis factor-alpha, Bcl-2, Bcl-X1 or caspase.

50. The method of claim 43 wherein the functional variant or mimetic comprises a conservative amino acid substitution or peptide mimetic substitution.

51. The method of claim 43 wherein the functional variant comprises about 70% or greater amino acid sequence identity to X₁-HGKHEERQDE-X₂ (SEQ ID NO:85).


SEQUENCE LISTING (967 sequences)

SEQ ID NO: 1 - protein, 8 aa, Homo sapiens
MDIAIHHP

SEQ ID NO: 2 - protein, 8 aa, Homo sapiens
IAIHHPWI

SEQ ID NO: 3 - protein, 8 aa, Homo sapiens
IHHPWIRR

SEQ ID NO: 4 - protein, 8 aa, Homo sapiens
HPWIRRPF

SEQ ID NO: 5 - protein, 8 aa, Homo sapiens
WIRRPFFP

SEQ ID NO: 6 - protein, 8 aa, Homo sapiens
RRPFFPFH

SEQ ID NO: 7 - protein, 8 aa, Homo sapiens
PFFPFHSP

SEQ ID NO: 8 - protein, 8 aa, Homo sapiens
FPFHSPSR

SEQ ID NO: 9 - protein, 8 aa, Homo sapiens
FHSPSRLF

SEQ ID NO: 10 - protein, 8 aa, Homo sapiens
SPSRLFDQ

SEQ ID NO: 11 - protein, 8 aa, Homo sapiens
SRLFDQFF

SEQ ID NO: 12 - protein, 8 aa, Homo sapiens
LFDQFFGE

SEQ ID NO: 13 - protein, 8 aa, Homo sapiens
DQFFGEHL

SEQ ID NO: 14 - protein, 8 aa, Homo sapiens
FFGEHLLE

SEQ ID NO: 15 - protein, 8 aa, Homo sapiens
GEHLLESD

SEQ ID NO: 16 - protein, 8 aa, Homo sapiens
HLLESDLF

SEQ ID NO: 17 - protein, 8 aa, Homo sapiens
LESDLFPT

SEQ ID NO: 18 - protein, 8 aa, Homo sapiens
SDLFPTST

SEQ ID NO: 19 - protein, 8 aa, Homo sapiens
LFPTSTSL

SEQ ID NO: 20 - protein, 8 aa, Homo sapiens
PTSTSLSP

SEQ ID NO: 21 - protein, 8 aa, Homo sapiens
STSLSPFY

SEQ ID NO: 22 - protein, 8 aa, Homo sapiens
SLSPFYLR

SEQ ID NO: 23 - protein, 8 aa, Homo sapiens
SPFYLRPP

SEQ ID NO: 24 - protein, 8 aa, Homo sapiens
FYLRPPSF

SEQ ID NO: 25 - protein, 8 aa, Homo sapiens
LRPPSFLR

SEQ ID NO: 26 - protein, 8 aa, Homo sapiens
PPSFLRAP

SEQ ID NO: 27 - protein, 8 aa, Homo sapiens
SFLRAPSW

SEQ ID NO: 28 - protein, 8 aa, Homo sapiens
LRAPSWFD

SEQ ID NO: 29 - protein, 8 aa, Homo sapiens
APSWFDTG

SEQ ID NO: 30 - protein, 8 aa, Homo sapiens
SWFDTGLS

SEQ ID NO: 31 - protein, 8 aa, Homo sapiens
FDTGLSEM

SEQ ID NO: 32 - protein, 8 aa, Homo sapiens
TGLSEMRL

SEQ ID NO: 33 - protein, 8 aa, Homo sapiens
LSEMRLEK

SEQ ID NO: 34 - protein, 8 aa, Homo sapiens
EMRLEKDR

SEQ ID NO: 35 - protein, 8 aa, Homo sapiens
RLEKDRFS

SEQ ID NO: 36 - protein, 8 aa, Homo sapiens
EKDRFSVN

SEQ ID NO: 37 - protein, 8 aa, Homo sapiens
DRFSVNLD

SEQ ID NO: 38 - protein, 8 aa, Homo sapiens
FSVNLDVK

SEQ ID NO: 39 - protein, 8 aa, Homo sapiens
VNLDVKHF

SEQ ID NO: 40 - protein, 8 aa, Homo sapiens
LDVKHFSP

SEQ ID NO: 41 - protein, 8 aa, Homo sapiens
VKHFSPEE

SEQ ID NO: 42 - protein, 8 aa, Homo sapiens
HFSPEELK

SEQ ID NO: 43 - protein, 8 aa, Homo sapiens
SPEELKVK

SEQ ID NO: 44 - protein, 8 aa, Homo sapiens
EELKVKVL

SEQ ID NO: 45 - protein, 8 aa, Homo sapiens
LKVKVLGD

SEQ ID NO: 46 - protein, 8 aa, Homo sapiens
VKVLGDVI

SEQ ID NO: 47 - protein, 8 aa, Homo sapiens
VLGDVIEV

SEQ ID NO: 48 - protein, 8 aa, Homo sapiens
GDVIEVHG

SEQ ID NO: 49 - protein, 8 aa, Homo sapiens
VIEVHGKH

SEQ ID NO: 50 - protein, 8 aa, Homo sapiens
EVHGKHEE

SEQ ID NO: 51 - protein, 8 aa, Homo sapiens
HGKHEERQ

SEQ ID NO: 52 - protein, 8 aa, Homo sapiens
KHEERQDE

SEQ ID NO: 53 - protein, 8 aa, Homo sapiens
EERQDEHG

SEQ ID NO: 54 - protein, 8 aa, Homo sapiens
RQDEHGFI

SEQ ID NO: 55 - protein, 8 aa, Homo sapiens
DEHGFISR

SEQ ID NO: 56 - protein, 8 aa, Homo sapiens
HGFISREF

SEQ ID NO: 57 - protein, 8 aa, Homo sapiens
FISREFHR

SEQ ID NO: 58 - protein, 8 aa, Homo sapiens
SREFHRKY

SEQ ID NO: 59 - protein, 8 aa, Homo sapiens
EFHRKYRI

SEQ ID NO: 60 - protein, 8 aa, Homo sapiens
HRKYRIPA

SEQ ID NO: 61 - protein, 8 aa, Homo sapiens
KYRIPADV

SEQ ID NO: 62 - protein, 8 aa, Homo sapiens
RIPADVDP

SEQ ID NO: 63 - protein, 8 aa, Homo sapiens
PADVDPLT

SEQ ID NO: 64 - protein, 8 aa, Homo sapiens
DVDPLTIT

SEQ ID NO: 65 - protein, 8 aa, Homo sapiens
DPLTITSS

SEQ ID NO: 66 - protein, 8 aa, Homo sapiens
LTITSSLS

SEQ ID NO: 67 - protein, 8 aa, Homo sapiens
ITSSLSSD

SEQ ID NO: 68 - protein, 8 aa, Homo sapiens
SSLSSDGV

SEQ ID NO: 69 - protein, 8 aa, Homo sapiens
LSSDGVLT

SEQ ID NO: 70 - protein, 8 aa, Homo sapiens
SDGVLTVN

SEQ ID NO: 71 - protein, 8 aa, Homo sapiens
GVLTVNGP

SEQ ID NO: 72 - protein, 8 aa, Homo sapiens
LTVNGPRK

SEQ ID NO: 73 - protein, 8 aa, Homo sapiens
VNGPRKQV

SEQ ID NO: 74 - protein, 8 aa, Homo sapiens
GPRKQVSG

SEQ ID NO: 75 - protein, 8 aa, Homo sapiens
RKQVSGPE

SEQ ID NO: 76 - protein, 8 aa, Homo sapiens
QVSGPERT

SEQ ID NO: 77 - protein, 8 aa, Homo sapiens
SGPERTIP

SEQ ID NO: 78 - protein, 8 aa, Homo sapiens
PERTIPIT

SEQ ID NO: 79 - protein, 8 aa, Homo sapiens
RTIPITRE

SEQ ID NO: 80 - protein, 8 aa, Homo sapiens
IPITREEK

SEQ ID NO: 81 - protein, 8 aa, Homo sapiens
ITREEKPA

SEQ ID NO: 82 - protein, 8 aa, Homo sapiens
REEKPAVT

SEQ ID NO: 83 - protein, 8 aa, Homo sapiens
EKPAVTAA

SEQ ID NO: 84 - protein, 8 aa, Homo sapiens
PAVTAAPK

SEQ ID NO: 85 - protein, 12 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (12)..(12): Xaa = each X1 or X2 represents any amino acid sequence of n amino acids, n varying from 0 to 50, and n being identical or different in X1 and X2
XHGKHEERQDEX

SEQ ID NO: 86 - protein, 6 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (6)..(6): Xaa = each X1 or X2 represents any amino acid sequence of n amino acids, n varying from 0 to 50, and n being identical or different in X1 and X2
XHGKHX

SEQ ID NO: 87 - protein, 6 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (6)..(6): Xaa = each X1 or X2 represents any amino acid sequence of n amino acids, n varying from 0 to 50, and n being identical or different in X1 and X2
XKHEEX

SEQ ID NO: 88 - protein, 8 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (8)..(8): Xaa = each X1 or X2 represents any amino acid sequence of n amino acids, n varying from 0 to 50, and n being identical or different in X1 and X2
XEERQDEX

SEQ ID NO: 89 - protein, 6 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (6)..(6): Xaa = each X1 or X2 represents any amino acid sequence of n amino acids, n varying from 0 to 50, and n being identical or different in X1 and X2
XRQDEX

SEQ ID NO: 90 - protein, 6 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (6)..(6): Xaa = each X1 or X2 represents any amino acid sequence of n amino acids, n varying from 0 to 50, and n being identical or different in X1 and X2
XEERQX

SEQ ID NO: 91 - protein, 8 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (8)..(8): Xaa = each X1 or X2 represents any amino acid sequence of n amino acids, n varying from 0 to 50, and n being identical or different in X1 and X2
XHGKHEEX

SEQ ID NO: 92 - protein, 8 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (8)..(8): Xaa = each X1 or X2 represents any amino acid sequence of n amino acids, n varying from 0 to 50, and n being identical or different in X1 and X2
XKHEERQX

SEQ ID NO: 93 - protein, 6 aa, Homo sapiens
  MISC_FEATURE (1)..(6): Xaa = each X1 or X2 represents any amino acid sequence of n amino acids, n varying from 0 to 50, and n being identical or different in X1 and X2
  MISC_FEATURE (6)..(6): Xaa = each X1 or X2 represents any amino acid sequence of n amino acids, n varying from 0 to 50, and n being identical or different in X1 and X2
XHEERX

SEQ ID NO: 94 - protein, 10 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (10)..(10): Xaa = each X1 or X2 represents any amino acid sequence of n amino acids, n varying from 0 to 50, and n being identical or different in X1 and X2
XHGKHEERQX

SEQ ID NO: 95 - protein, 10 aa, Homo sapiens
  MISC_FEATURE (1)..(1): Xaa = each X1 or X2 represents any amino acid sequence of n amino acids, n varying from 0 to 50, and n being identical or different in X1 and X2
  MISC_FEATURE (10)..(10): Xaa = each X1 or X2 represents any amino acid sequence of n amino acids, n varying from 0 to 50, and n being identical or different in X1 and X2
XKHEERQDEX

SEQ ID NO: 96 - protein, 17 aa, Homo sapiens
SLSPFYLRPPSFLRAPS

SEQ ID NO: 97 - protein, 15 aa, Homo sapiens
EKDRFSVNLDVKHFS

SEQ ID NO: 98 - protein, 13 aa, Homo sapiens
HGFISREFHRKYR

SEQ ID NO: 99 - protein, 23 aa, Homo sapiens
DPLTITSSLSSDGVLTVNGPRKQ

SEQ ID NO: 100 - protein, 12 aa, Homo sapiens
PERTIPITREEK

SEQ ID NO: 101 - protein, 13 aa, Homo sapiens
DRFSVNLDVKHFS

SEQ ID NO: 102 - protein, 11 aa, Homo sapiens
LTITSSLSDGV

SEQ ID NO: 103 - protein, 10 aa, Homo sapiens
HGKHEERQDE

SEQ ID NO: 104 - protein, 12 aa, Homo sapiens
WIRRPFFPFHSP

SEQ ID NO: 105 - protein, 16 aa, Homo sapiens
SLSPFYLRPPSFLRAP

SEQ ID NO: 106 - protein, 19 aa, Homo sapiens
LTITSSLSDGVLTVNGPRK

SEQ ID NO: 107 - protein, 18 aa, Homo sapiens
STSLSPFYLRPPSFLRAP

SEQ ID NO: 108 - protein, 13 aa, Homo sapiens
GPERTIPITREEK

SEQ ID NO: 109 - protein, 18 aa, Homo sapiens
LFPTSTSLSPFYLRPPSF

SEQ ID NO: 110 - protein, 19 aa, Homo sapiens
KFVIFLDVKHFSPEDLTVK

SEQ ID NO: 111 - protein, 13 aa, Homo sapiens
REEKPAVTAAPKK

SEQ ID NO: 112 - protein, 12 aa, Homo sapiens
SLSPFYLRPPSF

SEQ ID NO: 113 - protein, 16 aa, Homo sapiens
TSLSPFYLRPPSFLRA

SEQ ID NO: 114 - protein, 9 aa, Homo sapiens
ERTIPITRE

SEQ ID NO: 115 - protein, 12 aa, Homo sapiens
LTITSSLSSDGV

SEQ ID NO: 116 - protein, 4 aa, Homo sapiens
HGKH

SEQ ID NO: 117 - protein, 4 aa, Homo sapiens
KHEE

SEQ ID NO: 118 - protein, 6 aa, Homo sapiens
EERQDE

SEQ ID NO: 119 - protein, 4 aa, Homo sapiens
RQDE

SEQ ID NO: 120 - protein, 4 aa, Homo sapiens
EERQ

SEQ ID NO: 121 - protein, 6 aa, Homo sapiens
HGKHEE

SEQ ID NO: 122 - protein, 6 aa, Homo sapiens
KHEERQ

SEQ ID NO: 123 - protein, 4 aa, Homo sapiens
HEER

SEQ ID NO: 124 - protein, 11 aa, Homo sapiens
NAPVSIPQNAP

SEQ ID NO: 125 - protein, 10 aa, Homo sapiens
DRVYIHPFHL

SEQ ID NO: 126 - protein, 10 aa, Homo sapiens
DRFSVNLDVK

SEQ ID NO: 127 - protein, 17 aa, Homo sapiens
AASQHRHHPYNRGASAA

SEQ ID NO: 128 - protein, 17 aa, Homo sapiens
AGMPRQQPQPVQDGASQ

SEQ ID NO: 129 - protein, 15 aa, Homo sapiens
ALMERQFAPVCRISP

SEQ ID NO: 130 - protein, 15 aa, Homo sapiens
AQMERQFTPVCRGSP

SEQ ID NO: 131 - protein, 12 aa, Homo sapiens
ATMPVRLLRDSP

SEQ ID NO: 132 - protein, 17 aa, Homo sapiens
AVMPRFSSQLDRLYPGI

SEQ ID NO: 133 - protein, 14 aa, Homo sapiens
DAASTWDWPLQHND

SEQ ID NO: 134 - protein, 12 aa, Homo sapiens
DAFTNGFMNDIF

SEQ ID NO: 135 - protein, 13 aa, Homo sapiens
DCVGAGLLERAGR

SEQ ID NO: 136 - protein, 17 aa, Homo sapiens
DDFGSFMRPHSEAFPRP

SEQ ID NO: 137 - protein, 14 aa, Homo sapiens
DEGTKWDWPLQKGD

SEQ ID NO: 138 - protein, 16 aa, Homo sapiens
DFFDKMFDNFFSDDFF

SEQ ID NO: 139 - protein, 17 aa, Homo sapiens
DFFPLLRDSPGFDRLFR

SEQ ID NO: 140 - protein, 17 aa, Homo sapiens
DFSPLSRSSVGFEHLFD

SEQ ID NO: 141 - protein, 17 aa, Homo sapiens
DFSPLYRSTVGFDRLFS

SEQ ID NO: 142 - protein, 17 aa, Homo sapiens
DFSPLYRSTVGFDRLFT

SEQ ID NO: 143 - protein, 17 aa, Homo sapiens
DFTPLYRNAIGFDRLLN

SEQ ID NO: 144 - protein, 17 aa, Homo sapiens
DFWKQVFDTPFMTNGQS

SEQ ID NO: 145 - protein, 17 aa, Homo sapiens
DLAPLYRSAIGFDRLFN

SEQ ID NO: 146 - protein, 17 aa, Homo sapiens
DLSPFYRSSIGFDRIFN

SEQ ID NO: 147 - protein, 17 aa, Homo sapiens
DLSPLLRQWIGFDKLAN

SEQ ID NO: 148 - protein, 17 aa, Homo sapiens
DLSPLLRQWIGFDKLAS

SEQ ID NO: 149 - protein, 17 aa, Homo sapiens
DLSPLMRQWIGFDKLAN

SEQ ID NO: 150 - protein, 17 aa, Homo sapiens
DLSPLYRSAIGFDRLFN

SEQ ID NO: 151 - protein, 17 aa, Homo sapiens
DLSPLYRSLVGFDRLAD

SEQ ID NO: 152 - protein, 17 aa, Homo sapiens
DMMPYWAQRHSMNFNVP

SEQ ID NO: 153 - protein, 17 aa, Homo sapiens
DPFAIEPAGDIFQGLLR

SEQ ID NO: 154 - protein, 17 aa, Homo sapiens
DPFEELRRQERFNRLLE

SEQ ID NO: 155 - protein, 17 aa, Homo sapiens
DPFLSLHRNRLFDEVFR

SEQ ID NO: 156 - protein, 17 aa, Homo sapiens
DPFMGLHRSRLVDDMFR

SEQ ID NO: 157 - protein, 17 aa, Homo sapiens
DPFREIEETQRMDRAFA

SEQ ID NO: 158 - protein, 11 aa, Homo sapiens
DPFRELDRFAE

SEQ ID NO: 159 - protein, 17 aa, Homo sapiens
DQMPYWRRPLSRARPLS

SEQ ID NO: 160 - protein, 14 aa, Homo sapiens
DQTAKWDWPFQKGD

SEQ ID NO: 161 - protein, 17 aa, Homo sapiens
DRLFDDFASLWHRPLAS

SEQ ID NO: 162 - protein, 9 aa, Homo sapiens
DRLFDDFFR

SEQ ID NO: 163 - protein, 14 aa, Homo sapiens
DSAASWDWPLQHND

SEQ ID NO: 164 - protein, 17 aa, Homo sapiens
DYAPLFRNSIGFDRALN

SEQ ID NO: 165 - protein, 17 aa, Homo sapiens
DYAPLFRSSVGFDRVFN

SEQ ID NO: 166 - protein, 17 aa, Homo sapiens
DYTPLYRSTVGFDRLFD

SEQ ID NO: 167 - protein, 17 aa, Homo sapiens
EALLEEMGRPGEDPELR

SEQ ID NO: 168 - protein, 17 aa, Homo sapiens
EEDFRRFSGPDGKPVVR

SEQ ID NO: 169 - protein, 17 aa, Homo sapiens
EEIDAMFDRRWKEPELY

SEQ ID NO: 170 - protein, 17 aa, Homo sapiens
EEIDAMFDRRWREPEVY

SEQ ID NO: 171 - protein, 17 aa, Homo sapiens
EEIFRKAEGPDGKPEIR

SEQ ID NO: 172 - protein, 15 aa, Homo sapiens
EEVERLRRDFERLLE

SEQ ID NO: 173 - protein, 16 aa, Homo sapiens
EITNEYKQSDFWTNVL

SEQ ID NO: 174 - protein, 17 aa, Homo sapiens
EKLASRVQGPDGVPKVE

SEQ ID NO: 175 - protein, 15 aa, Homo sapiens
ENQNFFNGNPSDTFK

SEQ ID NO: 176 - protein, 12 aa, Homo sapiens
EPWDRWLRDFFG

SEQ ID NO: 177 - protein, 14 aa, Homo sapiens
ERLNQLFEDFMPME

SEQ ID NO: 178 - protein, 17 aa, Homo sapiens
ERLSGDMDRPGEEPEIR

SEQ ID NO: 179 - protein, 16 aa, Homo sapiens
ERMMEDMMADGQRLDR

SEQ ID NO: 180 - protein, 17 aa, Homo sapiens
ERSHGHHNQMSRRASGG

SEQ ID NO: 181 - protein, 17 aa, Homo sapiens
ETFSTMTRLREFEELLE

SEQ ID NO: 182 - protein, 14 aa, Homo sapiens
EYMEQMMRTFPALE

SEQ ID NO: 183 - protein, 13 aa, Homo sapiens
FATPMTGTTMIQS

SEQ ID NO: 184 - protein, 9 aa, Homo sapiens
FPEFSELFA

SEQ ID NO: 185 - protein, 17 aa, Homo sapiens
GGMQRRLMPISGTFNPM

SEQ ID NO: 186 - protein, 16 aa, Homo sapiens
GQVLALRREMANRNDI

SEQ ID NO: 187 - protein, 17 aa, Homo sapiens
GRLDREYVPISENNDLS

SEQ ID NO: 188 - protein, 16 aa, Homo sapiens
KFFTNEMIKNVSNTVK

SEQ ID NO: 189 - protein, 17 aa, Homo sapiens
KLFREDGREMEPEEITE

SEQ ID NO: 190 - protein, 15 aa, Homo sapiens
KNMREMQKEFEKKIA

SEQ ID NO: 191 - protein, 13 aa, Homo sapiens
KQVFDRFFEHNGD

SEQ ID NO: 192 - protein, 16 aa, Homo sapiens
LPPAAIESPAVAAPLS

SEQ ID NO: 193 - protein, 17 aa, Homo sapiens
LRMDDSAWCHGSLVGIE

SEQ ID NO: 194 - protein, 17 aa, Homo sapiens
LVSRRVRNQLIQTPYNY

SEQ ID NO: 195 - protein, 17 aa, Homo sapiens
LYHGYYVRPRAAAGESR

SEQ ID NO: 196 - protein, 16 aa, Homo sapiens
MIRPYWADQTMLGHRV

SEQ ID NO: 197 - protein, 17 aa, Homo sapiens
MRGFRGKGGPDGVPKIE

SEQ ID NO: 198 - protein, 17 aa, Homo sapiens
MRIWNRILGSDGKPIFQ

SEQ ID NO: 199 - protein, 15 aa, Homo sapiens
NNMATFNTTSSYNRT

SEQ ID NO: 200 - protein, 17 aa, Homo sapiens
NPFLALHRNRLFDDAFR

SEQ ID NO: 201 - protein, 16 aa, Homo sapiens
PNVYQDFLRSIDDFFS

SEQ ID NO: 202 - protein, 15 aa, Homo sapiens
PTVNDLFSDFVSYSP

SEQ ID NO: 203 - protein, 17 aa, Homo sapiens
QLANTPAKDETLDDWFD

SEQ ID NO: 204 - protein, 17 aa, Homo sapiens
QPYNAYSNTSSHAHRLK

SEQ ID NO: 205 - protein, 16 aa, Homo sapiens
QQLRQLEKQVGASSGS

SEQ ID NO: 206 - protein, 12 aa, Homo sapiens
QQMNQLFEEVFV

SEQ ID NO: 207 - protein, 17 aa, Homo sapiens
REANFFESTSSTSRIIS

SEQ ID NO: 208 - protein, 16 aa, Homo sapiens
RFFTNEMLKNVSNTVK

SEQ ID NO: 209 - protein, 15 aa, Homo sapiens
RHLDQMRREFDRFFP

SEQ ID NO: 210 - protein, 17 aa, Homo sapiens
RHVADLIDITNIDHLFN

SEQ ID NO: 211 - protein, 17 aa, Homo sapiens
RLSSAWPGTLRSPRGPA

SEQ ID NO: 212 - protein, 17 aa, Homo sapiens
RMGAHAHHLVANKRNAA

SEQ ID NO: 213 - protein, 17 aa, Homo sapiens
RQATPNSQPELEGKLFA

SEQ ID NO: 214 - protein, 11 aa, Homo sapiens
RQINRLFDRLM

SEQ ID NO: 215 - protein, 12 aa, Homo sapiens
RQLNHLFEEDML

SEQ ID NO: 216 - protein, 17 aa, Homo sapiens
RQVADLIDITNIDNLFK

SEQ ID NO: 217 - protein, 17 aa, Homo sapiens
SEDETMIALFGADPYRN

SEQ ID NO: 218 - protein, 17 aa, Homo sapiens
SFIPNFNDSNRFNQIDK

SEQ ID NO: 219 - protein, 17 aa, Homo sapiens
SGKPVLARLQQLMTLRE

SEQ ID NO: 220 - protein, 17 aa, Homo sapiens
SKRNDANDFDSMDEWLR

SEQ ID NO: 221 - protein, 17 aa, Homo sapiens
SLAPLFRHSVGFDRFND

SEQ ID NO: 222 - protein, 17 aa, Homo sapiens
SLSPFYLRPPSFLRAFD

SEQ ID NO: 223 - protein, 17 aa, Homo sapiens
SPAPAAVESPAVAAPLS

SEQ ID NO: 224 - protein, 17 aa, Homo sapiens
SSFAHLQASPIPDPLQV

SEQ ID NO: 225 - protein, 15 aa, Homo sapiens
TISPYYRQSLFRTLD

SEQ ID NO: 226 - protein, 17 aa, Homo sapiens
TIVDLRKTRAAQSPPDS

SEQ ID NO: 227 - protein, 15 aa, Homo sapiens
TLAPYYLRAPSVALP

SEQ ID NO: 228 - protein, 17 aa, Homo sapiens
TVFPTHRGPPDQTKIPV

SEQ ID NO: 229 - protein, 16 aa, Homo sapiens
VALPRNWQQIARWQEQ

SEQ ID NO: 230 - protein, 16 aa, Homo sapiens
VFADSLFSDRFNRIDR

SEQ ID NO: 231 - protein, 17 aa, Homo sapiens
VLRSGYLRPWHTSLQKQ

SEQ ID NO: 232 - protein, 17 aa, Homo sapiens
VQFPYWRNADHNSFNFS

SEQ ID NO: 233 - protein, 16 aa, Homo sapiens
WDLPYWKRSLSRVGSA

SEQ ID NO: 234 - protein, 15 aa, Homo sapiens
AEHGVRITLAVAGFS

SEQ ID NO: 235 - protein, 15 aa, Homo sapiens
AGTHLDLLLDVPGVD

SEQ ID NO: 236 - protein, 15 aa, Homo sapiens
ARDGFQMKLDAHGFA

SEQ ID NO: 237 - protein, 15 aa, Homo sapiens
DDDHFEVGLEAHNFL

SEQ ID NO: 238 - protein, 15 aa, Homo sapiens
DDDKYTVAADLPGVK

SEQ ID NO: 239 - protein, 15 aa, Homo sapiens
DDENLDIEIDMFGVK

SEQ ID NO: 240 - protein, 15 aa, Homo sapiens
DDKKVYLTFELPGVS

SEQ ID NO: 241 - protein, 15 aa, Homo sapiens
DDKKVYVTFELPGVS

SEQ ID NO: 242 - protein, 15 aa, Homo sapiens
DDNHYRITLALAGFR

SEQ ID NO: 243 - protein, 15 aa, Homo sapiens
DDRRFAVDMDCYQFR

SEQ ID NO: 244 - protein, 15 aa, Homo sapiens
DEEALELKVDMPGLA

SEQ ID NO: 245 - protein, 15 aa, Homo sapiens
DEEYNFIIVEMPGVY

SEQ ID NO: 246 - protein, 15 aa, Homo sapiens
DEKEVKMRFDMPGLS

SEQ ID NO: 247 - protein, 15 aa, Homo sapiens
DEKGFRIDIDVRQFH

SEQ ID NO: 248 - protein, 15 aa, Homo sapiens
DENEIKMRFDMPGLS

SEQ ID NO: 249 - protein, 15 aa, Homo sapiens
DENHYRIAIAVAGFA

SEQ ID NO: 250 - protein, 15 aa, Homo sapiens
DEQGLELTLDIPGVK

SEQ ID NO: 251 - protein, 15 aa, Homo sapiens
DESKFSVQLDVSHFK

SEQ ID NO: 252 - protein, 15 aa, Homo sapiens
DGDDAVVRLELPGID

SEQ ID NO: 253 - protein, 15 aa, Homo sapiens
DGKTLRLRFDVANYK

SEQ ID NO: 254 - protein, 15 aa, Homo sapiens
DGTDLVLEAEMPGFD

SEQ ID NO: 255 - protein, 15 aa, Homo sapiens
DKDKFAIHLDVKHFT

SEQ ID NO: 256 - protein, 15 aa, Homo sapiens
DNKVLKLRFDVSQYA

SEQ ID NO: 257 - protein, 15 aa, Homo sapiens
DPGHFSVLLDVKHFS

SEQ ID NO: 258 - protein, 15 aa, Homo sapiens
DRDKFTIMLDVKHFS

SEQ ID NO: 259 - protein, 15 aa, Homo sapiens
DRDKFVIFLDVKHFS

SEQ ID NO: 260 - protein, 15 aa, Homo sapiens
DSEKFEVILDVQQFS

SEQ ID NO: 261 - protein, 15 aa, Homo sapiens
DSSDLVLEAEMAGFD

SEQ ID NO: 262 - protein, 15 aa, Homo sapiens
EDDEFVLSVEMPGFD

SEQ ID NO: 263 - protein, 15 aa, Homo sapiens
EDDKIKVVVDMPGVE

SEQ ID NO: 264 - protein, 15 aa, Homo sapiens
EDDKLVVTTDLPGIN

SEQ ID NO: 265 - protein, 15 aa, Homo sapiens
EDNKVIVTTDLPGIN

SEQ ID NO: 266 - protein, 15 aa, Homo sapiens
EEHEIKMRFDMPGLS

SEQ ID NO: 267 - protein, 15 aa, Homo sapiens
EEHEIRMRFDMPGLA

SEQ ID NO: 268 - protein, 15 aa, Homo sapiens
EEHEIRMRFDMPGVS

SEQ ID NO: 269 - protein, 15 aa, Homo sapiens
EGEEFVVEFDLPGIK

SEQ ID NO: 270 - protein, 15 aa, Homo sapiens
EGEEVRIEIDMPGLE

SEQ ID NO: 271 - protein, 15 aa, Homo sapiens
EGEQIRVVADLPGFS

SEQ ID NO: 272 - protein, 15 aa, Homo sapiens
EGGELVVVADLAGFN

SEQ ID NO: 273 - protein, 15 aa, Homo sapiens
EKDKFAVRADVSHFH

SEQ ID NO: 274 - protein, 15 aa, Homo sapiens
EKDKFSVNLDVKHFS

SEQ ID NO: 275 - protein, 15 aa, Homo sapiens
EKDRFMINLNVKHFS

SEQ ID NO: 276 - protein, 15 aa, Homo sapiens
EKSHYRITLALAGFR

SEQ ID NO: 277 - protein, 15 aa, Homo sapiens
EMKRYEVRAELPGVD

SEQ ID NO: 278 - protein, 15 aa, Homo sapiens
EPNQFVLYADLPGID

SEQ ID NO: 279 - protein, 15 aa, Homo sapiens
GADAYRYVVDVAGVT

SEQ ID NO: 280 - protein, 15 aa, Homo sapiens
GDQHIKVIAWLPGVN

SEQ ID NO: 281 - protein, 15 aa, Homo sapiens
GEDHFKVRFDAQGFA

SEQ ID NO: 282 - protein, 15 aa, Homo sapiens
GKDGFEANVDVHLFK

SEQ ID NO: 283 - protein, 15 aa, Homo sapiens
GKDGFQVCMDVAQFK

SEQ ID NO: 284 - protein, 15 aa, Homo sapiens
GKDGFQVCMDVSHFK

SEQ ID NO: 285 - protein, 15 aa, Homo sapiens
GKDGFQVCMDVSQFK

SEQ ID NO: 286 - protein, 15 aa, Homo sapiens
GKDHFELTLNVRDFS

SEQ ID NO: 287 - protein, 15 aa, Homo sapiens
GKDTVSVDVELPGVK

SEQ ID NO: 288 - protein, 15 aa, Homo sapiens
GKSHFQILLDVVQFL

SEQ ID NO: 289 - protein, 15 aa, Homo sapiens
GRGTFKVVLDVHHFQ

SEQ ID NO: 290 - protein, 15 aa, Homo sapiens
GSPNVRVTTQTHTAI

SEQ ID NO: 291 - protein, 15 aa, Homo sapiens
HDNNYELKVVVPGVK

SEQ ID NO: 292 - protein, 15 aa, Homo sapiens
HGDEYRIVIAAAGFQ

SEQ ID NO: 293 - protein, 15 aa, Homo sapiens
HPNAYAFVVDMPGIK

SEQ ID NO: 294 - protein, 15 aa, Homo sapiens
HPNSYVFMVDMPGVK

SEQ ID NO: 295 - protein, 15 aa, Homo sapiens
IEGHIKVIAWLPGVN

SEQ ID NO: 296 - protein, 15 aa, Homo sapiens
IEHAYAFVVDMPGIK

SEQ ID NO: 297 - protein, 15 aa, Homo sapiens
IESAFELHADAPGMG

SEQ ID NO: 298 - protein, 15 aa, Homo sapiens
INEEYQLTLSIPGYQ

SEQ ID NO: 299 - protein, 15 aa, Homo sapiens
INEKFAVRADVSHFH

SEQ ID NO: 300 - protein, 15 aa, Homo sapiens
IRGRLRITLAVAGFS

SEQ ID NO: 301 - protein, 15 aa, Homo sapiens
KDDAIHLKLEVPGLE

SEQ ID NO: 302 - protein, 15 aa, Homo sapiens
KDKQYIIVMEVPGFD

SEQ ID NO: 303 - protein, 15 aa, Homo sapiens
KEDKYTVAADLPGVK

SEQ ID NO: 304 - protein, 15 aa, Homo sapiens
KEDQYRITMAVAGFG

SEQ ID NO: 305 - protein, 15 aa, Homo sapiens
KEEEIKMRFDMPGLS

SEQ ID NO: 306 - protein, 15 aa, Homo sapiens
KEGRYEVRAELPGVD

SEQ ID NO: 307 - protein, 15 aa, Homo sapiens
KELAYAFVVDMPGLG

SEQ ID NO: 308 - protein, 15 aa, Homo sapiens
KETAHVFKADLPGVK

SEQ ID NO: 309 - protein, 15 aa, Homo sapiens
KETAHVFKADVPGLK

SEQ ID NO: 310 - protein, 15 aa, Homo sapiens
KETGHVIMVDVPGLK

SEQ ID NO: 311 - protein, 15 aa, Homo sapiens
KGDEIKVVAEVPGVN

SEQ ID NO: 312 - protein, 15 aa, Homo sapiens
KQGNFEVHLDVGLFQ

SEQ ID NO: 313 - protein, 15 aa, Homo sapiens
LDHNYELKVVVPGVK

SEQ ID NO: 314 - protein, 15 aa, Homo sapiens
LGDAYEFAVDVRDFS

SEQ ID NO: 315 - protein, 15 aa, Homo sapiens
LGDRLRILIDLPGMD

SEQ ID NO: 316 - protein, 14 aa, Homo sapiens
LGEIVLYVDMPGIR

SEQ ID NO: 317 - protein, 15 aa, Homo sapiens
LGETYRIVLAVAGFG

SEQ ID NO: 318 - protein, 15 aa, Homo sapiens
LGGRLRITLAVAGFA

SEQ ID NO: 319 - protein, 15 aa, Homo sapiens
LGPDYRITMAIAGFS

SEQ ID NO: 320 - protein, 15 aa, Homo sapiens
LPGAYAFVVDMPGLG

SEQ ID NO: 321 - protein, 15 aa, Homo sapiens
LPGRLRITIAVAGFS

SEQ ID NO: 322 - protein, 15 aa, Homo sapiens
LRGRLRITLAVAGFA

SEQ ID NO: 323 - protein, 15 aa, Homo sapiens
NDQEYNVSVDVSQFE

SEQ ID NO: 324 - protein, 15 aa, Homo sapiens
NDQKFAINLNVSQFK

SEQ ID NO: 325 - protein, 15 aa, Homo sapiens
NENMVHVLAEFPEIE

SEQ ID NO: 326 - protein, 15 aa, Homo sapiens
NKDGYKLTLDVKDYS

SEQ ID NO: 327 - protein, 15 aa, Homo sapiens
NQDGVIITAELPGVR

SEQ ID NO: 328 - protein, 15 aa, Homo sapiens
NRNGFQVSMNVKQFA

SEQ ID NO: 329 - protein, 15 aa, Homo sapiens
PGEPWKVCVNVHSFK

SEQ ID NO: 330 - protein, 15 aa, Homo sapiens
RDANYLLTVSVPGWK

SEQ ID NO: 331 - protein, 15 aa, Homo sapiens
RDDEVWIVADIPGAN

SEQ ID NO: 332 - protein, 15 aa, Homo sapiens
RDKEIKVTAELPGLD

SEQ ID NO: 333 - protein, 15 aa, Homo sapiens
REDALELKVDMPGLA

SEQ ID NO: 334 - protein, 15 aa, Homo sapiens
RETAHVFKADVPGLK

SEQ ID NO: 335 - protein, 15 aa, Homo sapiens
RETAYIFKADLPGVD

SEQ ID NO: 336 - protein, 15 aa, Homo sapiens
RGDEFVVIAELPGVR

SEQ ID NO: 337 - protein, 15 aa, Homo sapiens
RGDELVVIAELPGVR

SEQ ID NO: 338 - protein, 15 aa, Homo sapiens
RSGEFVIVAEVPGAR

SEQ ID NO: 339 - protein, 15 aa, Homo sapiens
SADSWKVTLDVNHFA

SEQ ID NO: 340 - protein, 15 aa, Homo sapiens
SDDEYLLVFDTPGAD

SEQ ID NO: 341 - protein, 15 aa, Homo sapiens
SDDHYRITLALAGFR

SEQ ID NO: 342 - protein, 15 aa, Homo sapiens
SDTAYSVVAEIPGAK

SEQ ID NO: 343 - protein, 15 aa, Homo sapiens
SEAEVVVMIEVPGFS

SEQ ID NO: 344 - protein, 15 aa, Homo sapiens
SEGKFQAFLDVSHFT

SEQ ID NO: 345 - protein, 15 aa, Homo sapiens
SNDKFAVNLNVSNFK

SEQ ID NO: 346 - protein, 15 aa, Homo sapiens
SPTAFELHADAPGMG

SEQ ID NO: 347 - protein, 15 aa, Homo sapiens
TADRWRVSLDINYFA

SEQ ID NO: 348 - protein, 15 aa, Homo sapiens
TADRWRVSLDVNHFA

SEQ ID NO: 349 - protein, 15 aa, Homo sapiens
TAEVIYLKLELPGID

SEQ ID NO: 350 - protein, 15 aa, Homo sapiens
TAQKFCVKLDVAAFK

SEQ ID NO: 351 - protein, 15 aa, Homo sapiens
TDDAIIIKTDLPGVK

SEQ ID NO: 352 - protein, 15 aa, Homo sapiens
TDDEVVIEVEIPGID

SEQ ID NO: 353 - protein, 15 aa, Homo sapiens
TDDHYRISLALAGFK

SEQ ID NO: 354 - protein, 15 aa, Homo sapiens
TDEEIRLTAEIPGLD

SEQ ID NO: 355 - protein, 15 aa, Homo sapiens
TDELYYLEAELAGVN

SEQ ID NO: 356 - protein, 15 aa, Homo sapiens
TDHEVILECDIPGLE

SEQ ID NO: 357 - protein, 15 aa, Homo sapiens
TDKDIRVTAEIPGME

SEQ ID NO: 358 - protein, 15 aa, Homo sapiens
TDKEVVIKMDLPGVK

SEQ ID NO: 359 - protein, 15 aa, Homo sapiens
TDNEIQVIAEMPGVN

SEQ ID NO: 360 - protein, 15 aa, Homo sapiens
TEDAIRVIADLPGVE

SEQ ID NO: 361 - protein, 15 aa, Homo sapiens
TEDTIEVDVEVPGID

SEQ ID NO: 362 - protein, 15 aa, Homo sapiens
TEDTYVVVLALPGAN

SEQ ID NO: 363 - protein, 15 aa, Homo sapiens
TEEAYVLKLELPGMD

SEQ ID NO: 364 - protein, 15 aa, Homo sapiens
TEEKVHVIAEMPGIE

SEQ ID NO: 365 - protein, 15 aa, Homo sapiens
TEKTYEITCELPGME

SEQ ID NO: 366 - protein, 15 aa, Homo sapiens
TETGLVVVADLPGVG

SEQ ID NO: 367 - protein, 15 aa, Homo sapiens
TGDRYRIAIAVAGFE

SEQ ID NO: 368 - protein, 15 aa, Homo sapiens
TGDSYRIAIAVAGFA

SEQ ID NO: 369 - protein, 15 aa, Homo sapiens
TGEAYRIEIAVAGFK

SEQ ID NO: 370 - protein, 15 aa, Homo sapiens
TGEAYRITMAVAGFD

SEQ ID NO: 371 - protein, 15 aa, Homo sapiens
TGEAYRITMAVAGFS

SEQ ID NO: 372 - protein, 15 aa, Homo sapiens
TGESYRISMAVAGFS

SEQ ID NO: 373 - protein, 15 aa, Homo sapiens
THEKFSVNLNVPDVK

SEQ ID NO: 374 - protein, 15 aa, Homo sapiens
TKEAYIFKADLPGVD

SEQ ID NO: 375 - protein, 15 aa, Homo sapiens
TKEKFEVGLDVQFFT

SEQ ID NO: 376 - protein, 15 aa, Homo sapiens
TPEAHVFKADLPGVK

SEQ ID NO: 377 - protein, 15 aa, Homo sapiens
TPEAHVFKADVPGLK

SEQ ID NO: 378 - protein, 15 aa, Homo sapiens
TPEGHVIMVDVPGLK

SEQ ID NO: 379 - protein, 15 aa, Homo sapiens
TSEKFEVGLDAGFFG

SEQ ID NO: 380 - protein, 15 aa, Homo sapiens
TSHGFTIEIDVFHFM

SEQ ID NO: 381 - protein, 15 aa, Homo sapiens
TSQQYIIEADLTFLQ

SEQ ID NO: 382 - protein, 15 aa, Homo sapiens
TVAEVQFLIYLPGYR

SEQ ID NO: 383 - protein, 15 aa, Homo sapiens
VDEHYRIAIAVAGFA

SEQ ID NO: 384 - protein, 15 aa, Homo sapiens
VDENYRIAIAVAGFA

SEQ ID NO: 385 - protein, 15 aa, Homo sapiens
VGEQYRIAMSVAGFA

SEQ ID NO: 386 - protein, 15 aa, Homo sapiens
VNDKFSVQLDVSHFK

SEQ ID NO: 387 - protein, 15 aa, Homo sapiens
VNRGFQVSMNVKQFA

SEQ ID NO: 388 - protein, 15 aa, Homo sapiens
WREEFVVEFDLPGIK

SEQ ID NO: 389 - protein, 15 aa, Homo sapiens
YEDHFEVGLEAHNFL

SEQ ID NO: 390 - protein, 15 aa, Homo sapiens
YEKQFIIEAELPGVS

SEQ ID NO: 391 - protein, 15 aa, Homo sapiens
YPNSYVFIADMPGVK

SEQ ID NO: 392 - protein, 15 aa, Homo sapiens
YPNSYVFIIDMPGLK

SEQ ID NO: 393 - protein, 13 aa, Homo sapiens
RERAYGEFERTFR

SEQ ID NO: 394 - protein, 13 aa, Homo sapiens
EIPEEEEIYRTIK

SEQ ID NO: 395 - protein, 13 aa, Homo sapiens
RERRMGKVYRRIA

SEQ ID NO: 396 - protein, 13 aa, Homo sapiens
RGIAARQFQRVFV

SEQ ID NO: 397 - protein, 13 aa, Homo sapiens
RGIATRAFERRFQ

SEQ ID NO: 398 - protein, 13 aa, Homo sapiens
RGIAARSFEHRFE

SEQ ID NO: 399 - protein, 13 aa, Homo sapiens
RGIAGRPFEHRFE

SEQ ID NO: 400 - protein, 13 aa, Homo sapiens
EKFSFREYRGVYR

SEQ ID NO: 401 - protein, 13 aa, Homo sapiens
REREIGHFRRVVP

SEQ ID NO: 402 - protein, 13 aa, Homo sapiens
KSISSQARERVIP

SEQ ID NO: 403 - protein, 13 aa, Homo sapiens
RGIASRAFERRFQ

SEQ ID NO: 404 - protein, 13 aa, Homo sapiens
RERYYGEIERIIQ

SEQ ID NO: 405 - protein, 13 aa, Homo sapiens
KEGKYGSFEKKIP

SEQ ID NO: 406 - protein, 13 aa, Homo sapiens
KGIVFNNFSLNFN

SEQ ID NO: 407 - protein, 13 aa, Homo sapiens
RERSYGELRRSFY

SEQ ID NO: 408 - protein, 13 aa, Homo sapiens
RGLAERNFERKFQ

SEQ ID NO: 409 - protein, 13 aa, Homo sapiens
RGIAARGFVRTFV

SEQ ID NO: 410 - protein, 13 aa, Homo sapiens
VERAYGTFTRTFS

SEQ ID NO: 411 - protein, 13 aa, Homo sapiens
SERPSGRFVRELA

SEQ ID NO: 412 - protein, 13 aa, Homo sapiens
QGLMNQPFSLSFT

SEQ ID NO: 413 - protein, 13 aa, Homo sapiens
QGIAERNFERKFQ

SEQ ID NO: 414 - protein, 13 aa, Homo sapiens
KERAYTQFYRAVR

SEQ ID NO: 415 - protein, 13 aa, Homo sapiens
KERSFMRYYREIP

SEQ ID NO: 416 - protein, 13 aa, Homo sapiens
RERVTGEVRRRID

SEQ ID NO: 417 - protein, 13 aa, Homo sapiens
AERPRGVFNRQLV

SEQ ID NO: 418 - protein, 13 aa, Homo sapiens
REIRYGSFRRSFR

SEQ ID NO: 419 - protein, 13 aa, Homo sapiens
SEFAYGSFVRTVS

SEQ ID NO: 420 - protein, 13 aa, Homo sapiens
KERKYGEAKREMR

SEQ ID NO: 421 - protein, 13 aa, Homo sapiens
QGIAQRAFKLSFR

SEQ ID NO: 422 - protein, 13 aa, Homo sapiens
IERYYSGYRRVIR

SEQ ID NO: 423 - protein, 13 aa, Homo sapiens
ERISNFPVSRKIE

SEQ ID NO: 424 - protein, 13 aa, Homo sapiens
VERYYSGYRRVIR

SEQ ID NO: 425 - protein, 13 aa, Homo sapiens
DNYMNKNFNYVIS

SEQ ID NO: 426 - protein, 13 aa, Homo sapiens
SERYSGSMQRMFT

SEQ ID NO: 427 - protein, 13 aa, Homo sapiens
DERNFESLMRQFD

SEQ ID NO: 428 - protein, 13 aa, Homo sapiens
SERRYGSFQRTFR

SEQ ID NO: 429 - protein, 13 aa, Homo sapiens
SERYYGRFERRIA

SEQ ID NO: 430 - protein, 13 aa, Homo sapiens
RGIAARNFERRFQ

SEQ ID NO: 431 - protein, 13 aa, Homo sapiens
RGIAARQFQRTFV

SEQ ID NO: 432 - protein, 13 aa, Homo sapiens
RGIAKRAFERRFQ

SEQ ID NO: 433 - protein, 13 aa, Homo sapiens
SERRFGSFSRTIT

SEQ ID NO: 434 - protein, 13 aa, Homo sapiens
SERCVGAFSRTIT

SEQ ID NO: 435 - protein, 13 aa, Homo sapiens
ATQRPLKIHKVIR

SEQ ID NO: 436 - protein, 13 aa, Homo sapiens
RGIRKADFQLSFS

SEQ ID NO: 437 - protein, 13 aa, Homo sapiens
QGLVMQPFSLSFT

SEQ ID NO: 438 - protein, 13 aa, Homo sapiens
VERSAGKFERAIR

SEQ ID NO: 439 - protein, 12 aa, Homo sapiens
DQRVDKVYKVVK

SEQ ID NO: 440 - protein, 12 aa, Homo sapiens
DQRVDKVFKVVR

SEQ ID NO: 441 - protein, 13 aa, Homo sapiens
QGIAERDFERKFQ

SEQ ID NO: 442 - protein, 13 aa, Homo sapiens
GRGLTLEGHATLP

SEQ ID NO: 443 - protein, 13 aa, Homo sapiens
REDRSLFVDAELP

SEQ ID NO: 444 - protein, 13 aa, Homo sapiens
LRECPDRLERAIT

SEQ ID NO: 445 - protein, 13 aa, Homo sapiens
TRSQRKTYHRRFR

SEQ ID NO: 446 - protein, 13 aa, Homo sapiens
IERRYGSFHRRFA

SEQ ID NO: 447 - protein, 13 aa, Homo sapiens
KESSSGKFKRVIT

SEQ ID NO: 448 - protein, 13 aa, Homo sapiens
TELKYGAFERTVK

SEQ ID NO: 449 - protein, 13 aa, Homo sapiens
QGLVRKEFSLTFT

SEQ ID NO: 450 - protein, 13 aa, Homo sapiens
SEFQYGKFQRVIP

SEQ ID NO: 451 - protein, 13 aa, Homo sapiens
SEFRYGKFQRVIP

SEQ ID NO: 452 - protein, 13 aa, Homo sapiens
AERFYGVIERVIP

SEQ ID NO: 453 - protein, 13 aa, Homo sapiens
SERFYGRFERRIP

SEQ ID NO: 454 - protein, 13 aa, Homo sapiens
SERYYGRFERRFG

SEQ ID NO: 455 - protein, 13 aa, Homo sapiens
HGLALRSFARRFE

SEQ ID NO: 456 - protein, 13 aa, Homo sapiens
QGLAIGNFRQAFK

SEQ ID NO: 457 - protein, 13 aa, Homo sapiens
RGIAARQFQRCFV

SEQ ID NO: 458 - protein, 13 aa, Homo sapiens
RERRQGRFVRTVS

SEQ ID NO: 459 - protein, 13 aa, Homo sapiens
REYEVGDFERQFT

SEQ ID NO: 460 - protein, 13 aa, Homo sapiens
TEFRYGSFRRVIP

SEQ ID NO: 461 - protein, 13 aa, Homo sapiens
HGYVSRSFVRKYL

SEQ ID NO: 462 - protein, 13 aa, Homo sapiens
HGVISRHFIRKYI

SEQ ID NO: 463 - protein, 13 aa, Homo sapiens
HGYVSRQFSRRYQ

SEQ ID NO: 464 - protein, 13 aa, Homo sapiens
TKSVYREYNREFL

SEQ ID NO: 465 - protein, 13 aa, Homo sapiens
HGMIQRHFVRKYT

SEQ ID NO: 466 - protein, 13 aa, Homo sapiens
HGFITRHFVRRYA

SEQ ID NO: 467 - protein, 13 aa, Homo sapiens
HGHIMRHFVRRYK

SEQ ID NO: 468 - protein, 13 aa, Homo sapiens
NGLVERHFVRKYL

SEQ ID NO: 469 - protein, 13 aa, Homo sapiens
GGYSSRHFLRRFV

SEQ ID NO: 470 - protein, 13 aa, Homo sapiens
HGHVSRHFVRRYP

SEQ ID NO: 471 - protein, 13 aa, Homo sapiens
DTFVGRHIVKRFV

SEQ ID NO: 472 - protein, 13 aa, Homo sapiens
QLCITREFTRSYK

SEQ ID NO: 473 - protein, 13 aa, Homo sapiens
HGTVAREINRAYK

SEQ ID NO: 474 - protein, 13 aa, Homo sapiens
HGFSKRSFTRQFT

SEQ ID NO: 475 - protein, 13 aa, Homo sapiens
GHTLRRSFSRKYS

SEQ ID NO: 476 - protein, 13 aa, Homo sapiens
QRTVFREYNQEFL

SEQ ID NO: 477 - protein, 13 aa, Homo sapiens
FGSITRSITRCYR

SEQ ID NO: 478 - protein, 13 aa, Homo sapiens
FGDVSRNITRCYK

SEQ ID NO: 479 - protein, 13 aa, Homo sapiens
DNFTKMYFVRKYQ

SEQ ID NO: 480 - protein, 13 aa, Homo sapiens
YGQVERHFVRKYN

SEQ ID NO: 481 - protein, 13 aa, Homo sapiens
NISTTQTYSKSIV

SEQ ID NO: 482 - protein, 13 aa, Homo sapiens
HGASRKSFSRMIL

SEQ ID NO: 483 - protein, 13 aa, Homo sapiens
YGIVNREVHRTYK

SEQ ID NO: 484 - protein, 13 aa, Homo sapiens
HGYSKKSFSRVIL

SEQ ID NO: 485 - protein, 13 aa, Homo sapiens
HGYSKRSFSKMIL

SEQ ID NO: 486 - protein, 13 aa, Homo sapiens
HGYLKRSFSKMIL

SEQ ID NO: 487 - protein, 13 aa, Homo sapiens
YGTIESTFKRRFP

SEQ ID NO: 488 - protein, 13 aa, Homo sapiens
VSYRMSQKVHRKM

SEQ ID NO: 489 - protein, 13 aa, Homo sapiens
HGYISRCFTRKYT

SEQ ID NO: 490 - protein, 13 aa, Homo sapiens
HGFVAREFHRRYR

SEQ ID NO: 491 - protein, 13 aa, Homo sapiens
HGYISREFHRRYR

SEQ ID NO: 492 - protein, 13 aa, Homo sapiens
HGFVSREFCRTYV

SEQ ID NO: 493 - protein, 13 aa, Homo sapiens
HGFISRSFTRQYK

SEQ ID NO: 494 - protein, 13 aa, Homo sapiens
GGIVSKNFTKKIQ

SEQ ID NO: 495 - protein, 18 aa, Homo sapiens
AEDDIRAAYRNGVLEVRM

SEQ ID NO: 496 - protein, 18 aa, Homo sapiens
AEDKVAADFRNGVLSVSL

SEQ ID NO: 497 - protein, 23 aa, Homo sapiens
APESVQSQLTADGHLTIDAKAPE

SEQ ID NO: 498 - protein, 18 aa, Homo sapiens
DADGITASGSHGVLSIFI

SEQ ID NO: 499 - protein, 20 aa, Homo sapiens
DADNIKADYANGVLTLTVPK

SEQ ID NO: 500 - protein, 22 aa, Homo sapiens
DADNIKADYANGVLTLTVPKLK

SEQ ID NO: 501 - protein, 18 aa, Homo sapiens
DADRIEANFSNGLLTVTL

SEQ ID NO: 502 - protein, 18 aa, Homo sapiens
DAHSGAATYNNGILEVAF

SEQ ID NO: 503 - protein, 23 aa, Homo sapiens
DATQARATFSADGILMITVPAPP

SEQ ID NO: 504 - protein, 22 aa, Homo sapiens
DDSKIDASFLDGVLRITLPKKV

SEQ ID NO: 505 - protein, 20 aa, Homo sapiens
DDSQLKCRMTDGVLMLEAPV

SEQ ID NO: 506 - protein, 18 aa, Homo sapiens
DEDDIKATYDKGILTVSV

SEQ ID NO: 507 - protein, 20 aa, Homo sapiens
DEDDIKATYDKGILTVSVAV

SEQ ID NO: 508 - protein, 22 aa, Homo sapiens
DEDDIKATYDKGILTVSVAVSE

SEQ ID NO: 509 - protein, 18 aa, Homo sapiens
DEKAAKANFKNGVLEITL

SEQ ID NO: 510 - protein, 18 aa, Homo sapiens
DEKLIHASLNNGILSIVM

SEQ ID NO: 511 - protein, 18 aa, Homo sapiens
DENKVEATYESGLLRVTL

SEQ ID NO: 512 - protein, 18 aa, Homo sapiens
DESKVNATFRNGVLTVTL

SEQ ID NO: 513 - protein, 23 aa, Homo sapiens
DFNSIQSSIDAKGRLQVEAGKFN

SEQ ID NO: 514 - protein, 20 aa, Homo sapiens
DGDNVRADLKNGVLTLTLPK

SEQ ID NO: 515 - protein, 22 aa, Homo sapiens
DGDNVRADLKNGVLTLTLPKRP

SEQ ID NO: 516 - protein, 18 aa, Homo sapiens
DIDAVKAKYNNGVLEITI

SEQ ID NO: 517 - protein, 23 aa, Homo sapiens
DITSVATNLSNDGKLCIEAPKLE

SEQ ID NO: 518 - protein, 22 aa, Homo sapiens
DKDKVKAELKNGVLLISIPKTK

SEQ ID NO: 519 - protein, 22 aa, Homo sapiens
DKSQVRAELKNGVLLVSVPKRE

SEQ ID NO: 520 - protein, 23 aa, Homo sapiens
DLAHIHTVINKEGQMTIDAPKTG

SEQ ID NO: 521 - protein, 20 aa, Homo sapiens
DLDKISAVCHDGVLKVTVQK

SEQ ID NO: 522 - protein, 22 aa, Homo sapiens
DLDKISAVCHDGVLKVTVQKLP

SEQ ID NO: 523 - protein, 20 aa, Homo sapiens
DLDSVKAKMENGVLTLTLHK

SEQ ID NO: 524 - protein, 22 aa, Homo sapiens
DLDSVKAKMENGVLTLTLHKLS

SEQ ID NO: 525 - protein, 20 aa, Homo sapiens
DLPSVKSAISEGKLQIEAPK

SEQ ID NO: 526 - protein, 23 aa, Homo sapiens
DLPSVKSAISNEGKLQIEAPKKT

SEQ ID NO: 527 - protein, 18 aa, Homo sapiens
DLTKVEADFDHGTLNLRV

SEQ ID NO: 528 - protein, 23 aa, Homo sapiens
DLTSVKSAISNEGKLQIEAPKKT

SEQ ID NO: 529 - protein, 20 aa, Homo sapiens
DMDKISAVCRDGVLTVTVEK

SEQ ID NO: 530 - protein, 22 aa, Homo sapiens
DMDKISAVCRDGVLTVTVEKLP

SEQ ID NO: 531 - protein, 23 aa, Homo sapiens
DMKTIKSNLDSHGILHIEARKMH

SEQ ID NO: 532 - protein, 18 aa, Homo sapiens
DPAAVTSALSPEGVLSIQ

SEQ ID NO: 533 - protein, 23 aa, Homo sapiens
DPATIKSKLDGSGILHISGNKKK

SEQ ID NO: 534 - protein, 18 aa, Homo sapiens
DPDSAKARYNNGVLEVIL

SEQ ID NO: 535 - protein, 18 aa, Homo sapiens
DPEKAEAKYENGVLEIRI

SEQ ID NO: 536 - protein, 18 aa, Homo sapiens
DPGEATAEHVDGVCHVTV

SEQ ID NO: 537 - protein, 18 aa, Homo sapiens
DPKSAKASYKNGVLEVTF

SEQ ID NO: 538 - protein, 18 aa, Homo sapiens
DPKSAKASYRNGVLEIKL

SEQ ID NO: 539 - protein, 20 aa, Homo sapiens
DPLTITSSLSDGVLTVSAPR

SEQ ID NO: 540 - protein, 23 aa, Homo sapiens
DPLTITSSLSLDGVLTVSAPRKQ

SEQ ID NO: 541 - protein, 23 aa, Homo sapiens
DPLVITCSLSADGVLTITGPRKV

SEQ ID NO: 542 - protein, 20 aa, Homo sapiens
DPLVITCSLSDGVLTITGPR

SEQ ID NO: 543 - protein, 20 aa, Homo sapiens
DPNEVHSTLSDGILTVKAPQ

SEQ ID NO: 544 - protein, 23 aa, Homo sapiens
DPNEVHSTLSSDGILTVKAPPPL

SEQ ID NO: 545 - protein, 23 aa, Homo sapiens
DPNEVHSTLSSDGILTVKAPQPL

SEQ ID NO: 546 - protein, 20 aa, Homo sapiens
DPNEVVSTVSDGVLTLKAPP

SEQ ID NO: 547 - protein, 23 aa, Homo sapiens
DPNEVVSTVSSDGVLTLKAPPPP

SEQ ID NO: 548 - protein, 18 aa, Homo sapiens
DPNNVGANLINGLLTLGL

SEQ ID NO: 549 - protein, 18 aa, Homo sapiens
DPQSAKATYKNGVLEVTF

SEQ ID NO: 550 - protein, 18 aa, Homo sapiens
DPRSVRIRTRGSLIIVEA

SEQ ID NO: 551 - protein, 18 aa, Homo sapiens
DPSKAKATYKNGVLSIEL

SEQ ID NO: 552 - protein, 18 aa, Homo sapiens
DPSTATALYRHGVLIITA

SEQ ID NO: 553 - protein, 22 aa, Homo sapiens
DPSTVRSHLNSSGVLTITANKL

SEQ ID NO: 554 - protein, 23 aa, Homo sapiens
DPTKVSSSLSPEGTLTVEAPMPK

SEQ ID NO: 555 - protein, 23 aa, Homo sapiens
DPTSVTSALREDGSLTIRARRHP

SEQ ID NO: 556 - protein, 23 aa, Homo sapiens
DPVTVFASLSPEGLLIIEAPQVP

SEQ ID NO: 557 - protein, 23 aa, Homo sapiens
DPWRVRAALSHDGILNLEAPRGG

SEQ ID NO: 558 - protein, 18 aa, Homo sapiens
DQANISSSLKNGILTIIL

SEQ ID NO: 559 - protein, 16 aa, Homo sapiens
DQASISAKYQDGLLTV

SEQ ID NO: 560 - protein, 18 aa, Homo sapiens
DQKSISARLKNGILTIIL

SEQ ID NO: 561 - protein, 23 aa, Homo sapiens
DQSALSCSLSADGMLTFCGPKIQ

SEQ ID NO: 562 - protein, 23 aa, Homo sapiens
DQSAVTCVLSADGMLTFSGSKVQ

SEQ ID NO: 563 - protein, 20 aa, Homo sapiens
DQSAVTCVLSDGMLTFSGSK

SEQ ID NO: 564 - protein, 20 aa, Homo sapiens
DSDAIVSTLSDGVLNITVPP

SEQ ID NO: 565 - protein, 23 aa, Homo sapiens
DSDAIVSTLSEDGVLNITVPPLV

SEQ ID NO: 566 - protein, 17 aa, Homo sapiens
DSKIDASFLDGVLRITL

SEQ ID NO: 567 - protein, 19 aa, Homo sapiens
DSKIDASFLDGVLRITLPK

SEQ ID NO: 568 - protein, 18 aa, Homo sapiens
DTEKIAAKFSKSVLSITL

SEQ ID NO: 569 - protein, 18 aa, Homo sapiens
DTERILASYQEGVLKLSI

SEQ ID NO: 570 - protein, 20 aa, Homo sapiens
DTERILASYQEGVLKLSIPV

SEQ ID NO: 571 - protein, 22 aa, Homo sapiens
DTERILASYQEGVLKLSIPVAE

SEQ ID NO: 572 - protein, 18 aa, Homo sapiens
DVEKIKAEYKNGVLTIRV

SEQ ID NO: 573 - protein, 23 aa, Homo sapiens
DVGAVASNLSEDGKLSIEAPKKE

SEQ ID NO: 574 - protein, 23 aa, Homo sapiens
DVSTVKSHLATRGVLTITASKKA

SEQ ID NO: 575 - protein, 23 aa, Homo sapiens
DVTHLSSNLSEDGKLLIEVPKVE

SEQ ID NO: 576 - protein, 20 aa, Homo sapiens
EADKVASTLSDGVLTIKVPK

SEQ ID NO: 577 - protein, 23 aa, Homo sapiens
EADKVASTLSSDGVLTIKVPKPP

SEQ ID NO: 578 - protein, 23 aa, Homo sapiens
EADKVTSTLSSDGVLTISVPNPP

SEQ ID NO: 579 - protein, 20 aa, Homo sapiens
EATAVRSSLSDGMLTVEAPL

SEQ ID NO: 580 - protein, 23 aa, Homo sapiens
EATAVRSSLSPDGMLTVEAPLPK

SEQ ID NO: 581 - protein, 18 aa, Homo sapiens
EDDDIEAQYNNGILEVRL

SEQ ID NO: 582 - protein, 18 aa, Homo sapiens
EDDKVAATFKNGVLTVTL

SEQ ID NO: 583 - protein, 23 aa, Homo sapiens
EIKDLSAVLCHDGILVVEVKDPV

SEQ ID NO: 584 - protein, 16 aa, Homo sapiens
EIKSASLKNGLLHVDL

SEQ ID NO: 585 - protein, 18 aa, Homo sapiens
EINGIEANIKDGVLHLAI

SEQ ID NO: 586 - protein, 20 aa, Homo sapiens
EKDKIKAELKNGVLFITIPK

SEQ ID NO: 587 - protein, 22 aa, Homo sapiens
EKDKIKAELKNGVLFITIPKTK

SEQ ID NO: 588 - protein, 22 aa, Homo sapiens
EKDKVKAELKNGVLYITIPKTK

SEQ ID NO: 589 - protein, 23 aa, Homo sapiens
ENIHVRGANLVNGLLYIDLERVI

SEQ ID NO: 590 - protein, 23 aa, Homo sapiens
ENMEVSGATFVNGLLHIDLIRNE

SEQ ID NO: 591 - protein, 16 aa, Homo sapiens
EQQDISAKYSEGILTV

SEQ ID NO: 592 - protein, 17 aa, Homo sapiens
ETHLQIQLLNGLLHISY

SEQ ID NO: 593 - protein, 16 aa, Homo sapiens
EVKAASLANGLLNIDL

SEQ ID NO: 594 - protein, 16 aa, Homo sapiens
EVQTASLKNGLLHIDL

SEQ ID NO: 595 - protein, 16 aa, Homo sapiens
EVSGASLKNGLLHIDL

SEQ ID NO: 596 - protein, 16 aa, Homo sapiens
EVSGATFTNGLLHIDL

SEQ ID NO: 597 - protein, 16 aa, Homo sapiens
EVSGATFVNGLLHIDL

SEQ ID NO: 598 - protein, 18 aa, Homo sapiens
EVSGATFVNGLLHIDLIR

SEQ ID NO: 599 - protein, 16 aa, Homo sapiens
EVTAATLEHGLLHIDL

SEQ ID NO: 600 - protein, 16 aa, Homo sapiens
EVVGATLKNGLLFVDL

SEQ ID NO: 601 - protein, 18 aa, Homo sapiens
GVDDIKADYANGVLTLTV

SEQ ID NO: 602 - protein, 17 aa, Homo sapiens
HGKMNAIFKDGILYITI

SEQ ID NO: 603 - protein, 23 aa, Homo sapiens
HLDTIRSHLTNSGVLIINVSFSK

SEQ ID NO: 604 - protein, 16 aa, Homo sapiens
HVRGANLVNGLLYIDL

SEQ ID NO: 605 - protein, 18 aa, Homo sapiens
HVRGANLVNGLLYIDLER

SEQ ID NO: 606 - protein, 16 aa, Homo sapiens
HVRGANLVNGLLYIEL

SEQ ID NO: 607 - protein, 18 aa, Homo sapiens
IPEKAKAKYNNGVLEIRI

SEQ ID NO: 608 - protein, 23 aa, Homo sapiens
KAEQVVSQLSSDGVLTVSIPKPQ

SEQ ID NO: 609 - protein, 18 aa, Homo sapiens
KEEGSTAKMENGVLTISL

SEQ ID NO: 610 - protein, 18 aa, Homo sapiens
KEENASAKFENGVLSVIL

SEQ ID NO: 611 - protein, 20 aa, Homo sapiens
KEENASAKFENGVLSVILPK

SEQ ID NO: 612 - protein, 20 aa, Homo sapiens
KIDGIKAEMKNGVLKVTVPK

SEQ ID NO: 613 - protein, 22 aa, Homo sapiens
KIDGIKAEMKNGVLKVTVPKIK

SEQ ID NO: 614 - protein, 16 aa, Homo sapiens
KIKGANLVNGLLYIDL

SEQ ID NO: 615 - protein, 20 aa, Homo sapiens
KPEQIKASMENGVLTVTVPK

SEQ ID NO: 616 - protein, 21 aa, Homo sapiens
KPEQIKASMENGVLTVTVPKE

SEQ ID NO: 617 - protein, 18 aa, Homo sapiens
KPETASAGFSDGLLRITV

SEQ ID NO: 618 - protein, 18 aa, Homo sapiens
KPETATAKFNNGLLDVTV

SEQ ID NO: 619 - protein, 18 aa, Homo sapiens
KPETAVAKFNNGLLDVTV

SEQ ID NO: 620 - protein, 17 aa, Homo sapiens
KQHITASFENGLLTITL

SEQ ID NO: 621 - protein, 18 aa, Homo sapiens
KRDQVTAKYENGVLTIRI

SEQ ID NO: 622 - protein, 16 aa, Homo sapiens
KVARATLENGLLSVDL

SEQ ID NO: 623 - protein, 21 aa, Homo sapiens
KVDQVKAGMENGVLTVTVPKN

SEQ ID NO: 624 - protein, 20 aa, Homo sapiens
KVEEVKAGLENGVLTVTVPK

SEQ ID NO: 625 - protein, 21 aa, Homo sapiens
KVEEVKAGLENGVLTVTVPKA

SEQ ID NO: 626 - protein, 20 aa, Homo sapiens
KVEEVKASMENGVLSVTVPK

SEQ ID NO: 627 - protein, 21 aa, Homo sapiens
KVEEVKASMENGVLSVTVPKV

SEQ ID NO: 628 - protein, 16 aa, Homo sapiens
KVKKAELSLGLLKLDF

SEQ ID NO: 629 - protein, 16 aa, Homo sapiens
KVNNAKLEQGLLLVEI

SEQ ID NO: 630 - protein, 16 aa, Homo sapiens
KVTGATMEHGLLHIDL

SEQ ID NO: 631 - protein, 18 aa, Homo sapiens
NADGRAELNEDGTLHVFL

SEQ ID NO: 632 - protein, 23 aa, Homo sapiens
NANEVISDISSDGILTIKAPPPP

SEQ ID NO: 633 - protein, 18 aa, Homo sapiens
NDKNIQVECENQILTVAV

SEQ ID NO: 634 - protein, 20 aa, Homo sapiens
NESAIACSLSEGLLTLCCPK

SEQ ID NO: 635 - protein, 23 aa, Homo sapiens
NESAIACSLSNEGLLTLCCPKTR

SEQ ID NO: 636 - protein, 22 aa, Homo sapiens
NIEAISAISQDGVLTVTVNKLP

SEQ ID NO: 637 - protein, 22 aa, Homo sapiens
NKEKITAVCQDGVLTVTVENVP

SEQ ID NO: 638 - protein, 20 aa, Homo sapiens
NPDGITAAMDKGVLVVTVPK

SEQ ID NO: 639 - protein, 22 aa, Homo sapiens
NPDGITAAMDKGVLVVTVPKRE

SEQ ID NO: 640 - protein, 23 aa, Homo sapiens
NPDTVTSSLSSDGLLTIKAPMKA

SEQ ID NO: 641 - protein, 23 aa, Homo sapiens
NPEQISSTLSTDGVLTVEAPLPQ

SEQ ID NO: 642 - protein, 23 aa, Homo sapiens
NPEQVVCSLSKNGHLHIQAPRLA

SEQ ID NO: 643 - protein, 20 aa, Homo sapiens
NPEQVVCSLSNGHLHIQAPR

SEQ ID NO: 644 - protein, 23 aa, Homo sapiens
NPESIRSSLSKDGVLTVDAPLPA

SEQ ID NO: 645 - protein, 18 aa, Homo sapiens
NPSAVSAKCGRGLLIVRA

SEQ ID NO: 646 - protein, 16 aa, Homo sapiens
NVDNAQFENGLLHIDL

SEQ ID NO: 647 - protein, 22 aa, Homo sapiens
NVEAINAVYQDGVLQVTVEKLP

SEQ ID NO: 648 - protein, 18 aa, Homo sapiens
QEGDIKARLRNGLLTIAI

SEQ ID NO: 649 - protein, 18 aa, Homo sapiens
QNDKVQAEYKNGILRLTV

SEQ ID NO: 650 - protein, 18 aa, Homo sapiens
QNTEVKANYDAGILTLTL

SEQ ID NO: 651 - protein, 18 aa, Homo sapiens
QNTNVTAEYKDGILNLTL

SEQ ID NO: 652 - protein, 23 aa, Homo sapiens
QPDTIESHLSDKGVLTICANKTA

SEQ ID NO: 653 - protein, 20 aa, Homo sapiens
QPDTIESHLSKGVLTICANK

SEQ ID NO: 654 - protein, 16 aa, Homo sapiens
QVLGAELRNGLLAVDL

SEQ ID NO: 655 - protein, 16 aa, Homo sapiens
QVREARLRNGLLSIDL

SEQ ID NO: 656 - protein, 18 aa, Homo sapiens
RIKEIKATYNNGLLQIKV

SEQ ID NO: 657 - protein, 17 aa, Homo sapiens
RPAGVASLAGGVLTVRF

SEQ ID NO: 658 - protein, 23 aa, Homo sapiens
RPEQIKSELSNNGVLTVKYEKNQ

SEQ ID NO: 659 - protein, 18 aa, Homo sapiens
RVEDASAKFENGVLTVEL

SEQ ID NO: 660 - protein, 18 aa, Homo sapiens
RVESAKAVYKDGVLQIVV

SEQ ID NO: 661 - protein, 16 aa, Homo sapiens
RVIAAELKNGLLSIDL

SEQ ID NO: 662 - protein, 16 aa, Homo sapiens
RVLACELRNGLLAIDL

SEQ ID NO: 663 - protein, 16 aa, Homo sapiens
RVNGADLKNGLLSIDL

SEQ ID NO: 664 - protein, 16 aa, Homo sapiens
RVSAAELKNGLLSVNL

SEQ ID NO: 665 - protein, 23 aa, Homo sapiens
SPTAMTCCLTPSGQLWVRGQCVA

SEQ ID NO: 666 - protein, 18 aa, Homo sapiens
TADGATATVSNGVLTVSL

SEQ ID NO: 667 - protein, 18 aa, Homo sapiens
TEEGATAQLKNGVLTVTM

SEQ ID NO: 668 - protein, 16 aa, Homo sapiens
TIVGAKLENGLLIIDL

SEQ ID NO: 669 - protein, 18 aa, Homo sapiens
TSEAIAASYDAGVLTVRV

SEQ ID NO: 670 - protein, 16 aa, Homo sapiens
TVTGANLANGLLKIDL

SEQ ID NO: 671 - protein, 18 aa, Homo sapiens
VPEKAVAKYVDGKLYVKV

SEQ ID NO: 672 - protein, 16 aa, Homo sapiens
VVVDADLSNGLLSIAL

SEQ ID NO: 673 - protein, 23 aa, Homo sapiens
YPNDVRSELSSDGILTVKCPPYL

SEQ ID NO: 674 - protein, 5 aa, Homo sapiens
AAPAS

SEQ ID NO: 675 - protein, 9 aa, Homo sapiens
AAPASAQAP

SEQ ID NO: 676 - protein, 8 aa, Homo sapiens
AAQRIAIS

SEQ ID NO: 677 - protein, 9 aa, Homo sapiens
ADIHVTATD

SEQ ID NO: 678 - protein, 8 aa, Homo sapiens
AERAIPVS

SEQ ID NO: 679 - protein, 12 aa, Homo sapiens
AERAIPVSREEK

SEQ ID NO: 680 - protein, 9 aa, Homo sapiens
AETQAQRIA

SEQ ID NO: 681 - protein, 9 aa, Homo sapiens
AKPKRIAIN

SEQ ID NO: 682 - protein, 9 aa, Homo sapiens
AKPRRIEIT

SEQ ID NO: 683 - protein, 8 aa, Homo sapiens
ALTEKRIP

SEQ ID NO: 684 - protein, 9 aa, Homo sapiens
ANAKRIAIN

SEQ ID NO: 685 - protein, 8 aa, Homo sapiens
ASRNIPIR

SEQ ID NO: 686 - protein, 8 aa, Homo sapiens
ASSGTEQK

SEQ ID NO: 687 - protein, 9 aa, Homo sapiens
DNGRRIDIH

SEQ ID NO: 688 - protein, 8 aa, Homo sapiens
DVKSIQIT

SEQ ID NO: 689 - protein, 8 aa, Homo sapiens
EAQTGPSP

SEQ ID NO: 690 - protein, 12 aa, Homo sapiens
EAQTGPSPRLGS

SEQ ID NO: 691 - protein, 9 aa, Homo sapiens
EKPKKIAIE

SEQ ID NO: 692 - protein, 9 aa, Homo sapiens
EKPKKISIN

SEQ ID NO: 693 - protein, 8 aa, Homo sapiens
EPKRIAVT

SEQ ID NO: 694 - protein, 11 aa, Homo sapiens
ERSVYVRQVGP

SEQ ID NO: 695 - protein, 9 aa, Homo sapiens
ETLIPIAHK

SEQ ID NO: 696 - protein, 9 aa, Homo sapiens
ETTSSTSIP

SEQ ID NO: 697 - protein, 8 aa, Homo sapiens
EVKAIQIS

SEQ ID NO: 698 - protein, 8 aa, Homo sapiens
EVKSVDIS

SEQ ID NO: 699 - protein, 8 aa, Homo sapiens
FGFLSKFR

SEQ ID NO: 700 - protein, 12 aa, Homo sapiens
FGFLSKFRCMPE

SEQ ID NO: 701 - protein, 8 aa, Homo sapiens
FNNELPQD

SEQ ID NO: 702 - protein, 12 aa, Homo sapiens
FNNELPQDSQEV

SEQ ID NO: 703 - protein, 8 aa, Homo sapiens
GARPIQIK

SEQ ID NO: 704 - protein, 12 aa, Homo sapiens
GARPIQIKVINT

SEQ ID NO: 705 - protein, 8 aa, Homo sapiens
GERLVRVH

SEQ ID NO: 706 - protein, 12 aa, Homo sapiens
GERLVRVHETGK

SEQ ID NO: 707 - protein, 9 aa, Homo sapiens
GKNHVKKIE

SEQ ID NO: 708 - protein, 8 aa, Homo sapiens
GPRMVSIV

SEQ ID NO: 709 - protein, 11 aa, Homo sapiens
GRSIPIQQAIV

SEQ ID NO: 710 - protein, 11 aa, Homo sapiens
GRSIPIQQAPV

SEQ ID NO: 711 - protein, 11 aa, Homo sapiens
GRSVPVKEASM

SEQ ID NO: 712 - protein, 8 aa, Homo sapiens
HVKKIEVS

SEQ ID NO: 713 - protein, 9 aa, Homo sapiens
IAAQRIAIS

SEQ ID NO: 714 - protein, 9 aa, Homo sapiens
IAPQRIAIN

SEQ ID NO: 715 - protein, 9 aa, Homo sapiens
IEEPKKKIE

SEQ ID NO: 716 - protein, 9 aa, Homo sapiens
IEVKPMEEE

SEQ ID NO: 717 - protein, 9 aa, Homo sapiens
KEGEGFEVK

SEQ ID NO: 718 - protein, 8 aa, Homo sapiens
KEREVTIE

SEQ ID NO: 719 - protein, 12 aa, Homo sapiens
KEREVTIEQTGE

SEQ ID NO: 720 - protein, 8 aa, Homo sapiens
KERIIPIK

SEQ ID NO: 721 - protein, 12 aa, Homo sapiens
KERIIPIKHVGP

SEQ ID NO: 722 - protein, 8 aa, Homo sapiens
KERIIQIQ

SEQ ID NO: 723 - protein, 12 aa, Homo sapiens
KERIIQIQQVGP

SEQ ID NO: 724 - protein, 8 aa, Homo sapiens
KERRIQIQ

SEQ ID NO: 725 - protein, 8 aa, Homo sapiens
KKDVFQVM

SEQ ID NO: 726 - protein, 9 aa, Homo sapiens
KKPKRIEIE

SEQ ID NO: 727 - protein, 9 aa, Homo sapiens
KKPRRIEIN

SEQ ID NO: 728 - protein, 9 aa, Homo sapiens
KPKPKKRIA

SEQ ID NO: 729 - protein, 8 aa, Homo sapiens
KPKTIEVK

SEQ ID NO: 730 - protein, 8 aa, Homo sapiens
KPKTIQVK

SEQ ID NO: 731 - protein, 8 aa, Homo sapiens
KPKTIQVQ

SEQ ID NO: 732 - protein, 8 aa, Homo sapiens
KPKTVEVK

SEQ ID NO: 733 - protein, 8 aa, Homo sapiens
KPRKISVD

SEQ ID NO: 734 - protein, 8 aa, Homo sapiens
KPRRIEIN

SEQ ID NO: 735 - protein, 8 aa, Homo sapiens
KPRTIEVK

SEQ ID NO: 736 - protein, 9 aa, Homo sapiens
KRIEVRSVS

SEQ ID NO: 737 - protein, 9 aa, Homo sapiens
KRSISVRSG

SEQ ID NO: 738 - protein, 8 aa, Homo sapiens
KVIDVQIQ

SEQ ID NO: 739 - protein, 8 aa, Homo sapiens
KVIDVQVQ

SEQ ID NO: 740 - protein, 8 aa, Homo sapiens
KVTDVEIK

SEQ ID NO: 741 - protein, 9 aa, Homo sapiens
LKPQKIDIQ

SEQ ID NO: 742 - protein, 9 aa, Homo sapiens
LKPRKIAIT

SEQ ID NO: 743 - protein, 9 aa, Homo sapiens
LKPRRIAIG

SEQ ID NO: 744 - protein, 9 aa, Homo sapiens
LKPRRIEIK

SEQ ID NO: 745 - protein, 9 aa, Homo sapiens
LKPRTVEIK

SEQ ID NO: 746 - protein, 9 aa, Homo sapiens
LQPQRIAIG

SEQ ID NO: 747 - protein, 9 aa, Homo sapiens
MKPRKIEVT

SEQ ID NO: 748 - protein, 9 aa, Homo sapiens
MKPRRIAIN

SEQ ID NO: 749 - protein, 9 aa, Homo sapiens
MKPRRIAIS

SEQ ID NO: 750 - protein, 9 aa, Homo sapiens
MKPRRIEIH

SEQ ID NO: 751 - protein, 9 aa, Homo sapiens
MQPRKIAIN

SEQ ID NO: 752 - protein, 9 aa, Homo sapiens
MRPRKIAIE

SEQ ID NO: 753 - protein, 8 aa, Homo sapiens
NEITIPVT

SEQ ID NO: 754 - protein, 12 aa, Homo sapiens
NEITIPVTFESR

SEQ ID NO: 755 - protein, 8 aa, Homo sapiens
NEITLESR

SEQ ID NO: 756 - protein, 8 aa, Homo sapiens
NERIVQIQ

SEQ ID NO: 757 - protein, 12 aa, Homo sapiens
NERIVQIQQVGP

SEQ ID NO: 758 - protein, 8 aa, Homo sapiens
NERSIPIE

SEQ ID NO: 759 - protein, 12 aa, Homo sapiens
NERSIPIEQVGP

SEQ ID NO: 760 - protein, 8 aa, Homo sapiens
NEVYISLL

SEQ ID NO: 761 - protein, 12 aa, Homo sapiens
NEVYISLLPAPP

SEQ ID NO: 762 - protein, 8 aa, Homo sapiens
NGRRIDIH

SEQ ID NO: 763 - protein, 9 aa, Homo sapiens
NKPRRIEIN

SEQ ID NO: 764 - protein, 8 aa, Homo sapiens
PERSIPIT

SEQ ID NO: 765 - protein, 8 aa, Homo sapiens
PERSVPIS

SEQ ID NO: 766 - protein, 8 aa, Homo sapiens
PETPIPIS

SEQ ID NO: 767 - protein, 11 aa, Homo sapiens
PKSIPITIVPK

SEQ ID NO: 768 - protein, 9 aa, Homo sapiens
PRKISVDRG

SEQ ID NO: 769 - protein, 9 aa, Homo sapiens
PRRIQVGNA

SEQ ID NO: 770 - protein, 8 aa, Homo sapiens
QDRPIPVS

SEQ ID NO: 771 - protein, 8 aa, Homo sapiens
QERIVDIQ

SEQ ID NO: 772 - protein, 12 aa, Homo sapiens
QERIVDIQQISQ

SEQ ID NO: 773 - protein, 8 aa, Homo sapiens
QGRSIPIQ

SEQ ID NO: 774 - protein, 9 aa, Homo sapiens
QNERKIQIK

SEQ ID NO: 775 - protein, 8 aa, Homo sapiens
QVKAINVY

SEQ ID NO: 776 - protein, 8 aa, Homo sapiens
RERMIPIE

SEQ ID NO: 777 - protein, 12 aa, Homo sapiens
RERMIPIEGAGH

SEQ ID NO: 778 - protein, 8 aa, Homo sapiens
SDRPIPVA

SEQ ID NO: 779 - protein, 8 aa, Homo sapiens
SEITIPVT

SEQ ID NO: 780 - protein, 8 aa, Homo sapiens
SERIVQIQ

SEQ ID NO: 781 - protein, 12 aa, Homo sapiens
SERIVQIQQTGP

SEQ ID NO: 782 - protein, 9 aa, Homo sapiens
SFSLQFPLS

SEQ ID NO: 783 - protein, 9 aa, Homo sapiens
SKAKRIAIN

SEQ ID NO: 784 - protein, 9 aa, Homo sapiens
SKAKRIPIG

SEQ ID NO: 785 - protein, 11 aa, Homo sapiens
SRSIPINFVAK

SEQ ID NO: 786 - protein, 8 aa, Homo sapiens
SSRSIPIN

SEQ ID NO: 787 - protein, 8 aa, Homo sapiens
TEKHIQIR

SEQ ID NO: 788 - protein, 9 aa, Homo sapiens
TEKHIQIRS

SEQ ID NO: 789 - protein, 8 aa, Homo sapiens
TERLVQIT

SEQ ID NO: 790 - protein, 12 aa, Homo sapiens
TERLVQITQTGP

SEQ ID NO: 791 - protein, 9 aa, Homo sapiens
TRGKQIEVQ

SEQ ID NO: 792 - protein, 8 aa, Homo sapiens
TVIDVQIQ

SEQ ID NO: 793 - protein, 8 aa, Homo sapiens
TYSRVLVK

SEQ ID NO: 794 - protein, 12 aa, Homo sapiens
TYSRVLVKDGVR

SEQ ID NO: 795 - protein, 7 aa, Homo sapiens
VAELKID

SEQ ID NO: 796 - protein, 7 aa, Homo sapiens
VAFNKGL

SEQ ID NO: 797 - protein, 12 aa, Homo sapiens
VEREIEIEPTGN

SEQ ID NO: 798 - protein, 8 aa, Homo sapiens
VQQTFRTE

SEQ ID NO: 799 - protein, 11 aa, Homo sapiens
VQQTFRTEIKI

SEQ ID NO: 800 - protein, 11 aa, Homo sapiens
VRALPIHTSAG

SEQ ID NO: 801 - protein, 9 aa, Homo sapiens
VTARPAPGD

SEQ ID NO: 802 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 803 - protein, 17 aa, Homo sapiens
SLSPFYPLRPPSFLRAP

SEQ ID NO: 804 - protein, 17 aa, Homo sapiens
YEVHHQKLVFFAEDVGS

SEQ ID NO: 805 - protein, 17 aa, Homo sapiens
PFHEHAEVVFTANDSGP

SEQ ID NO: 806 - protein, 16 aa, Homo sapiens
EKVEHSDLSFSKDWSF

SEQ ID NO: 807 - protein, 17 aa, Homo sapiens
AGSIAAATGFVKKDQLG

SEQ ID NO: 808 - protein, 17 aa, Homo sapiens
NFLVHSSNNFGAILSST

SEQ ID NO: 809 - protein, 6 aa, Homo sapiens
AAEHED

SEQ ID NO: 810 - protein, 10 aa, Homo sapiens
AGKRDSGRSD

SEQ ID NO: 811 - protein, 6 aa, Homo sapiens
AGNIED

SEQ ID NO: 812 - protein, 7 aa, Homo sapiens
DIETLNG

SEQ ID NO: 813 - protein, 8 aa, Homo sapiens
EAHRRKSV

SEQ ID NO: 814 - protein, 10 aa, Homo sapiens
EAIVRREKEL

SEQ ID NO: 815 - protein, 10 aa, Homo sapiens
EAKHEKRRDG

SEQ ID NO: 816 - protein, 10 aa, Homo sapiens
EATVRREKEL

SEQ ID NO: 817 - protein, 8 aa, Homo sapiens
EGAKTEND

SEQ ID NO: 818 - protein, 9 aa, Homo sapiens
EGHHEVKTE

SEQ ID NO: 819 - protein, 10 aa, Homo sapiens
EGHHKERADS

SEQ ID NO: 820 - protein, 9 aa, Homo sapiens
EGIQEKKSE

SEQ ID NO: 821 - protein, 9 aa, Homo sapiens
EGIQETKSE

SEQ ID NO: 822 - protein, 10 aa, Homo sapiens
EGKHEEKEDE

SEQ ID NO: 823 - protein, 10 aa, Homo sapiens
EGKHEEKQDE

SEQ ID NO: 824 - protein, 10 aa, Homo sapiens
EGKHEEREDD

SEQ ID NO: 825 - protein, 10 aa, Homo sapiens
EGKHEEREDG

SEQ ID NO: 826 - protein, 10 aa, Homo sapiens
EGKHEERQDD

SEQ ID NO: 827 - protein, 10 aa, Homo sapiens
EGKHNEKTDK

SEQ ID NO: 828 - protein, 10 aa, Homo sapiens
EGKQADDRKG

SEQ ID NO: 829 - protein, 10 aa, Homo sapiens
EGKSEQQEEQ

SEQ ID NO: 830 - protein, 10 aa, Homo sapiens
EGNHEEREDD

SEQ ID NO: 831 - protein, 10 aa, Homo sapiens
EGQHDEKEDG

SEQ ID NO: 832 - protein, 10 aa, Homo sapiens
EGRHEDIRDK

SEQ ID NO: 833 - protein, 10 aa, Homo sapiens
EGRIKRSKEV

SEQ ID NO: 834 - protein, 10 aa, Homo sapiens
EGSHGEKEDT

SEQ ID NO: 835 - protein, 6 aa, Homo sapiens
EIKVLG

SEQ ID NO: 836 - protein, 7 aa, Homo sapiens
EIRAEGE

SEQ ID NO: 837 - protein, 10 aa, Homo sapiens
GQQQLDVRER

SEQ ID NO: 838 - protein, 10 aa, Homo sapiens
HAKHEDRQDD

SEQ ID NO: 839 - protein, 10 aa, Homo sapiens
HAKHEDRQDG

SEQ ID NO: 840 - protein, 9 aa, Homo sapiens
HAKHEEKSD

SEQ ID NO: 841 - protein, 9 aa, Homo sapiens
HAKHEEKTP

SEQ ID NO: 842 - protein, 10 aa, Homo sapiens
HAKHEERLDE

SEQ ID NO: 843 - protein, 10 aa, Homo sapiens
HARHEERPDE

SEQ ID NO: 844 - protein, 10 aa, Homo sapiens
HCRHETRSDN

SEQ ID NO: 845 - protein, 10 aa, Homo sapiens
HGKHAERQED

SEQ ID NO: 846 - protein, 10 aa, Homo sapiens
HGKHDERQDD

SEQ ID NO: 847 - protein, 10 aa, Homo sapiens
HGKHGERQDD

SEQ ID NO: 848 - protein, 10 aa, Homo sapiens
HGKHNERQDD

SEQ ID NO: 849 - protein, 10 aa, Homo sapiens
HGKHNERQVD

SEQ ID NO: 850 - protein, 10 aa, Homo sapiens
HGKHSDRQDD

SEQ ID NO: 851 - protein, 10 aa, Homo sapiens
HGKHSERQDD

SEQ ID NO: 852 - protein, 10 aa, Homo sapiens
HGQHEERQDE

SEQ ID NO: 853 - protein, 10 aa, Homo sapiens
HLRHDNRPTE

SEQ ID NO: 854 - protein, 10 aa, Homo sapiens
HMAHTTKDDK

SEQ ID NO: 855 - protein, 10 aa, Homo sapiens
HMEHNVKKDS

SEQ ID NO: 856 - protein, 9 aa, Homo sapiens
IASPATPEG

SEQ ID NO: 857 - protein, 10 aa, Homo sapiens
ISGEREEKSD

SEQ ID NO: 858 - protein, 10 aa, Homo sapiens
ISGEREEKTD

SEQ ID NO: 859 - protein, 10 aa, Homo sapiens
ISGEREKEGA

SEQ ID NO: 860 - protein, 10 aa, Homo sapiens
ISGEREKEGV

SEQ ID NO: 861 - protein, 10 aa, Homo sapiens
ISGEREREDA

SEQ ID NO: 862 - protein, 10 aa, Homo sapiens
ISGQREEKND

SEQ ID NO: 863 - protein, 10 aa, Homo sapiens
KAEHQEIEND

SEQ ID NO: 864 - protein, 10 aa, Homo sapiens
KAEKATDEKT

SEQ ID NO: 865 - protein, 10 aa, Homo sapiens
KAEKKEEFEE

SEQ ID NO: 866 - protein, 8 aa, Homo sapiens
KAERTEQK

SEQ ID NO: 867 - protein, 9 aa, Homo sapiens
KAERTEQKD

SEQ ID NO: 868 - protein, 10 aa, Homo sapiens
KAQHGTRMDE

SEQ ID NO: 869 - protein, 9 aa, Homo sapiens
KGAHADEQK

SEQ ID NO: 870 - protein, 10 aa, Homo sapiens
KGAHAEQKER

SEQ ID NO: 871 - protein, 10 aa, Homo sapiens
KGEHKEESGK

SEQ ID NO: 872 - protein, 9 aa, Homo sapiens
KGEKSEEKG

SEQ ID NO: 873 - protein, 9 aa, Homo sapiens
KGEQKKEDS

SEQ ID NO: 874 - protein, 10 aa, Homo sapiens
KGERKDEGNG

SEQ ID NO: 875 - protein, 10 aa, Homo sapiens
KGERKTESSS

SEQ ID NO: 876 - protein, 10 aa, Homo sapiens
KGGHKQEHGG

SEQ ID NO: 877 - protein, 10 aa, Homo sapiens
KGKHEEEEDD

SEQ ID NO: 878 - protein, 10 aa, Homo sapiens
KGKIEDRVGI

SEQ ID NO: 879 - protein, 10 aa, Homo sapiens
KGQKADETAS

SEQ ID NO: 880 - protein, 8 aa, Homo sapiens
KGQRFSYF

SEQ ID NO: 881 - protein, 8 aa, Homo sapiens
KGTITPVF

SEQ ID NO: 882 - protein, 10 aa, Homo sapiens
KGTPEPKEEK

SEQ ID NO: 883 - protein, 9 aa, Homo sapiens
KGTPEQPEN

SEQ ID NO: 884 - protein, 9 aa, Homo sapiens
KGTPEQPKE

SEQ ID NO: 885 - protein, 10 aa, Homo sapiens
KSDHREENGG

SEQ ID NO: 886 - protein, 10 aa, Homo sapiens
KSEAEEQRRR

SEQ ID NO: 887 - protein, 10 aa, Homo sapiens
LVSGEEESKD

SEQ ID NO: 888 - protein, 6 aa, Homo sapiens
NAEGEK

SEQ ID NO: 889 - protein, 6 aa, Homo sapiens
NAEREI

SEQ ID NO: 890 - protein, 10 aa, Homo sapiens
NAKRDDETKD

SEQ ID NO: 891 - protein, 10 aa, Homo sapiens
NAKRDKDDNN

SEQ ID NO: 892 - protein, 9 aa, Homo sapiens
QAERRYSRG

SEQ ID NO: 893 - protein, 10 aa, Homo sapiens
QAQHEERQDE

SEQ ID NO: 894 - protein, 8 aa, Homo sapiens
QATRSAKY

SEQ ID NO: 895 - protein, 9 aa, Homo sapiens
QGEHDVENE

SEQ ID NO: 896 - protein, 9 aa, Homo sapiens
QGEQELKTD

SEQ ID NO: 897 - protein, 9 aa, Homo sapiens
QGEQELKTE

SEQ ID NO: 898 - protein, 10 aa, Homo sapiens
QGKHGERQDD

SEQ ID NO: 899 - protein, 10 aa, Homo sapiens
QGKKENSDTQ

SEQ ID NO: 900 - protein, 10 aa, Homo sapiens
QGNHNRRDGS

SEQ ID NO: 901 - protein, 6 aa, Homo sapiens
RAEGAR

SEQ ID NO: 902 - protein, 8 aa, Homo sapiens
RAEKLAAD

SEQ ID NO: 903 - protein, 10 aa, Homo sapiens
RAERKEEKEE

SEQ ID NO: 904 - protein, 6 aa, Homo sapiens
RAERKR

SEQ ID NO: 905 - protein, 8 aa, Homo sapiens
RAERPGVD

SEQ ID NO: 906 - protein, 10 aa, Homo sapiens
RAERPVDPDR

SEQ ID NO: 907 - protein, 6 aa, Homo sapiens
RAKREE

SEQ ID NO: 908 - protein, 10 aa, Homo sapiens
RAKRSPMITE

SEQ ID NO: 909 - protein, 10 aa, Homo sapiens
RGEHKQGEGG

SEQ ID NO: 910 - protein, 10 aa, Homo sapiens
RGEHRDQDAG

SEQ ID NO: 911 - protein, 9 aa, Homo sapiens
RGEKHSETD

SEQ ID NO: 912 - protein, 9 aa, Homo sapiens
RGEKKAETE

SEQ ID NO: 913 - protein, 10 aa, Homo sapiens
RGEKQEEKKE

SEQ ID NO: 914 - protein, 9 aa, Homo sapiens
RGEKRAETE

SEQ ID NO: 915 - protein, 10 aa, Homo sapiens
RGEREEEEEE

SEQ ID NO: 916 - protein, 8 aa, Homo sapiens
RGERKPEE

SEQ ID NO: 917 - protein, 9 aa, Homo sapiens
RGKPTPVEK

SEQ ID NO: 918 - protein, 7 aa, Homo sapiens
RGRQIET

SEQ ID NO: 919 - protein, 7 aa, Homo sapiens
RGRQQEQ

SEQ ID NO: 920 - protein, 7 aa, Homo sapiens
RGRQTDD

SEQ ID NO: 921 - protein, 9 aa, Homo sapiens
RGSHANEPA

SEQ ID NO: 922 - protein, 10 aa, Homo sapiens
RIHEEEEMRE

SEQ ID NO: 923 - protein, 10 aa, Homo sapiens
RVDRFREFDA

SEQ ID NO: 924 - protein, 6 aa, Homo sapiens
SAERKR

SEQ ID NO: 925 - protein, 10 aa, Homo sapiens
SAKTGEEKET

SEQ ID NO: 926 - protein, 10 aa, Homo sapiens
SAKVERSEKK

SEQ ID NO: 927 - protein, 5 aa, Homo sapiens
SAQSG

SEQ ID NO: 928 - protein, 10 aa, Homo sapiens
SARHPQRLDR

SEQ ID NO: 929 - protein, 10 aa, Homo sapiens
SARIREHVVK

SEQ ID NO: 930 - protein, 10 aa, Homo sapiens
SASKGKEEEN

SEQ ID NO: 931 - protein, 10 aa, Homo sapiens
SAVFEEEVEI

SEQ ID NO: 932 - protein, 10 aa, Homo sapiens
SGDRQDTHST

SEQ ID NO: 933 - protein, 10 aa, Homo sapiens
SGDRQDTHST

SEQ ID NO: 934 - protein, 10 aa, Homo sapiens
SGEKKIEREQ

SEQ ID NO: 935 - protein, 10 aa, Homo sapiens
SGERFESTGD

SEQ ID NO: 936 - protein, 10 aa, Homo sapiens
SGERKKEEEK

SEQ ID NO: 937 - protein, 10 aa, Homo sapiens
SGERKSETKT

SEQ ID NO: 938 - protein, 6 aa, Homo sapiens
SGERPG

SEQ ID NO: 939 - protein, 10 aa, Homo sapiens
SGERQAEAED

SEQ ID NO: 940 - protein, 10 aa, Homo sapiens
SGEVVNKNES

SEQ ID NO: 941 - protein, 9 aa, Homo sapiens
SGGKREKST

SEQ ID NO: 942 - protein, 10 aa, Homo sapiens
SGKHEEKQQE

SEQ ID NO: 943 - protein, 10 aa, Homo sapiens
SGKREREESE

SEQ ID NO: 944 - protein, 10 aa, Homo sapiens
TAERSNQMGI

SEQ ID NO: 945 - protein, 7 aa, Homo sapiens
TANIPKM

SEQ ID NO: 946 - protein, 8 aa, Homo sapiens
TGAKAGED

SEQ ID NO: 947 - protein, 10 aa, Homo sapiens
TGERKKEAGG

SEQ ID NO: 948 - protein, 10 aa, Homo sapiens
TGERKSETKT

SEQ ID NO: 949 - protein, 8 aa, Homo sapiens
TGKEQETA

SEQ ID NO: 950 - protein, 10 aa, Homo sapiens
TGKHEEKQDE

SEQ ID NO: 951 - protein, 10 aa, Homo sapiens
TGKHERKSDT

SEQ ID NO: 952 - protein, 10 aa, Homo sapiens
TGKHTEETVE

SEQ ID NO: 953 - protein, 6 aa, Homo sapiens
TGNIEE

SEQ ID NO: 954 - protein, 8 aa, Homo sapiens
TGQKHEAS

SEQ ID NO: 955 - protein, 9 aa, Homo sapiens
TGQKKPEEG

SEQ ID NO: 956 - protein, 9 aa, Homo sapiens
TGRKAANDE

SEQ ID NO: 957 - protein, 7 aa, Homo sapiens
TIDAKGK

SEQ ID NO: 958 - protein, 7 aa, Homo sapiens
TLNVDED

SEQ ID NO: 959 - protein, 10 aa, Homo sapiens
TSKHEERQDE

SEQ ID NO: 960 - protein, 6 aa, Homo sapiens
TVKSSG

SEQ ID NO: 961 - protein, 6 aa, Homo sapiens
VIKAEN

SEQ ID NO: 962 - protein, 7 aa, Homo sapiens
VITAKSE

SEQ ID NO: 963 - protein, 10 aa, Homo sapiens
VSGERDKKGD

SEQ ID NO: 964 - protein, 10 aa, Homo sapiens
VSGEREDKND

SEQ ID NO: 965 - protein, 10 aa, Homo sapiens
VSGEREKDGV

SEQ ID NO: 966 - protein, 10 aa, Homo sapiens
VSGERENEGV

SEQ ID NO: 967 - protein, 9 aa, Homo sapiens
YGRVKEDTP